SEQUENCE LISTING (28 sequences)

SEQ ID NO: 1 - protein, 9 aa, synthetic construct
  REGION 1..9: note = Synthetic
GYSITSGYY

SEQ ID NO: 2 - protein, 7 aa, synthetic construct
  REGION 1..7: note = Synthetic
ITFDGAN

SEQ ID NO: 3 - protein, 12 aa, synthetic construct
  REGION 1..12: note = Synthetic
TRSSYDYDVLDY

SEQ ID NO: 4 - protein, 6 aa, synthetic construct
  REGION 1..6: note = Synthetic
QDISNF

SEQ ID NO: 5 - skipped

SEQ ID NO: 6 - protein, 9 aa, synthetic construct
  REGION 1..9: note = Synthetic
QQGHTLPYT

SEQ ID NO: 7 - protein, 6 aa, synthetic construct
  REGION 1..6: note = Synthetic
SGYYWN

SEQ ID NO: 8 - protein, 16 aa, synthetic construct
  REGION 1..16: note = Synthetic
YITFDGANNYNPSLKN

SEQ ID NO: 9 - protein, 10 aa, synthetic construct
  REGION 1..10: note = Synthetic
SSYDYDVLDY

SEQ ID NO: 10 - protein, 11 aa, synthetic construct
  REGION 1..11: note = Synthetic
RASQDISNFLN

SEQ ID NO: 11 - protein, 7 aa, synthetic construct
  REGION 1..7: note = Synthetic
YTSRLHS

SEQ ID NO: 12 - protein, 8 aa, synthetic construct
  REGION 1..8: note = Synthetic
GYSITSGY

SEQ ID NO: 13 - skipped

SEQ ID NO: 14 - protein, 8 aa, synthetic construct
  REGION 1..8: note = Synthetic
SYDYDVLD

SEQ ID NO: 15 - protein, 7 aa, synthetic construct
  REGION 1..7: note = Synthetic
SQDISNF

SEQ ID NO: 16 - protein, 6 aa, synthetic construct
  REGION 1..6: note = Synthetic
GHTLPY

SEQ ID NO: 17 - protein, 119 aa, synthetic construct
  REGION 1..119: note = Synthetic
QVQLQESGPGLVKPSQTLSLTCTVTGYSITSGYYWNWIRQPPGKGLEWIGYITFDGANNYNPSLKNRVSISRDTSKNQFSLKLSSVTAEDTATYYCTRSSYDYDVLDYWGQGTTVTVSS

SEQ ID NO: 18 - protein, 107 aa, synthetic construct
  REGION 1..107: note = Synthetic
DIQMTQSPSSLSASVGDRVTITCRASQDISNFLNWYQQKPGQPVKLLIYYTSRLHSGVPSRFSGSGSGTDFTLTISSLQPEDFATYYCQQGHTLPYTFGQGTKLEIK

SEQ ID NO: 19 - protein, 227 aa, synthetic construct
  REGION 1..227: note = Synthetic
QVQLQESGPGLVKPSQTLSLTCTVTGYSITSGYYWNWIRQPPGKGLEWIGYITFDGANNYNPSLKNRVSISRDTSKNQFSLKLSSVTAEDTATYYCTRSSYDYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 20 - protein, 214 aa, synthetic construct
  REGION 1..214: note = Synthetic
DIQMTQSPSSLSASVGDRVTITCRASQDISNFLNWYQQKPGQPVKLLIYYTSRLHSGVPSRFSGSGSGTDFTLTISSLQPEDFATYYCQQGHTLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - RNA, 21 nt, synthetic construct
  misc_feature 1..16: note = Synthetic
  source 1..21: mol_type = other RNA
ttcttccgtgaaattctggca

SEQ ID NO: 22 - RNA, 23 nt, synthetic construct
  source 1..23: mol_type = other RNA
tgccagaatttcacggaagaaca

SEQ ID NO: 23 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 24 - RNA, 23 nt, synthetic construct
  source 1..23: mol_type = other RNA
tgttcttccgtgaaattctggca

SEQ ID NO: 25 - DNA, 1574 bp, Homo sapiens
  source 1..1574: mol_type = genomic DNA
atggccctcccgacaccctcggacagcaccctccccgcggaagcccggggacgaggacggcgacggagactcgtttggaccccgagccaaagcgaggccctgcgagcctgctttgagcggaacccgtacccgggcatcgccaccagagaacggctggcccaggccatcggcattccggagcccagggtccagatttggtttcagaatgagaggtcacgccagctgaggcagcaccggcgggaatctcggccctggcccgggagacgcggcccgccagaaggccggcgaaagcggaccgccgtcaccggatcccagaccgccctgctcctccgagcctttgagaaggatcgctttccaggcatcgccgcccgggaggagctggccagagagacgggcctcccggagtccaggattcagatctggtttcagaatcgaagggccaggcacccgggacagggtggcagggcgcccgcgcaggcaggcggcctgtgcagcgcggcccccggcgggggtcaccctgctccctcgtgggtcgccttcgcccacaccggcgcgtggggaacggggcttcccgcaccccacgtgccctgcgcgcctggggctctcccacagggggctttcgtgagccaggcagcgagggccgcccccgcgctgcagcccagccaggccgcgccggcagaggggatctcccaacctgccccggcgcgcggggatttcgcctacgccgccccggctcctccggacggggcgctctcccaccctcaggctcctcgctggcctccgcacccgggcaaaagccgggaggaccgggacccgcagcgcgacggcctgccgggcccctgcgcggtggcacagcctgggcccgctcaagcggggccgcagggccaaggggtgcttgcgccacccacgtcccaggggagtccgtggtggggctggggccggggtccccaggtcgccggggcggcgtgggaaccccaagccggggcagctccacctccccagcccgcgcccccggacgcctccgcctccgcgcggcaggggcagatgcaaggcatcccggcgccctcccaggcgctccaggagccggcgccctggtctgcactcccctgcggcctgctgctggatgagctcctggcgagcccggagtttctgcagcaggcgcaacctctcctagaaacggaggccccgggggagctggaggcctcggaagaggccgcctcgctggaagcacccctcagcgaggaagaataccgggctctgctggaggagctttaggacgcggggtctaggcccggtgagagactccacaccgcggagaactgccattctttcctgggcatcccggggatcccagagccggcccaggtaccagcagacctgcgcgcagtgcgcaccccggctgacgtgcaagggagctcgctggcctctctgtgcccttgttcttccgtgaaattctggctgaatgtctccccccaccttccgacgctgtctaggcaaacctggattagagttacatctcctggatgattagttcagagatatattaaaatgccccctccctgtggatcctatag

SEQ ID NO: 26 - DNA, 1710 bp, Homo sapiens
  source 1..1710: mol_type = genomic DNA
atggccctcccgacaccctcggacagcaccctccccgcggaagcccggggacgaggacggcgacggagactcgtttggaccccgagccaaagcgaggccctgcgagcctgctttgagcggaacccgtacccgggcatcgccaccagagaacggctggcccaggccatcggcattccggagcccagggtccagatttggtttcagaatgagaggtcacgccagctgaggcagcaccggcgggaatctcggccctggcccgggagacgcggcccgccagaaggccggcgaaagcggaccgccgtcaccggatcccagaccgccctgctcctccgagcctttgagaaggatcgctttccaggcatcgccgcccgggaggagctggccagagagacgggcctcccggagtccaggattcagatctggtttcagaatcgaagggccaggcacccgggacagggtggcagggcgcccgcgcaggcaggcggcctgtgcagcgcggcccccggcgggggtcaccctgctccctcgtgggtcgccttcgcccacaccggcgcgtggggaacggggcttcccgcaccccacgtgccctgcgcgcctggggctctcccacagggggctttcgtgagccaggcagcgagggccgcccccgcgctgcagcccagccaggccgcgccggcagaggggatctcccaacctgccccggcgcgcggggatttcgcctacgccgccccggctcctccggacggggcgctctcccaccctcaggctcctcggtggcctccgcacccgggcaaaagccgggaggaccgggacccgcagcgcgacggcctgccgggcccctgcgcggtggcacagcctgggcccgctcaagcggggccgcagggccaaggggtgcttgcgccacccacgtcccaggggagtccgtggtggggctggggccggggtccccaggtcgccggggcggcgtgggaaccccaagccggggcagctccacctccccagcccgcgcccccggacgcctccgcctccgcgcggcaggggcagatgcaaggcatcccggcgccctcccaggcgctccaggagccggcgccctggtctgcactcccctgcggcctgctgctggatgagctcctggcgagcccggagtttctgcagcaggcgcaacctctcctagaaacggaggccccgggggagctggaggcctcggaagaggccgcctcgctggaagcacccctcagcgaggaagaataccgggctctgctggaggagctttaggacgcggggttgggacggggtcgggtggttcggggcagggcggtggcctctctttcgcggggaacacctggctggctacggaggggcgtgtctccgccccgccccctccaccgggctgaccggcctgggattcctgccttctaggtctaggcccggtgagagactccactccgcggagaactgcctttctttcctgggcatcccggggatcccagagccggcccaggtaccagcagacctgcgcgcagtgcgcaccccggctgacgtgcaagggagctcgctggcctctctgtgcccttgttcttccgtgaaattctggctgaatgtctccccccaccttccgacgctgtctaggcaaacctggattagagttacatctcctggatgattagttcagagatatattaaaatgccccctccctgtggatcctatag

SEQ ID NO: 27 - DNA, 2024 bp, Mus musculus
  source 1..2024: mol_type = genomic DNA
atggcagaagctggcagccctgttggtggcagtggtgtggcacgggaatcccggcggcgcaggaagacggtttggcaggcctggcaagagcaggccctgctatcaactttcaagaagaagagatacctgagcttcaaggagaggaaggagctggccaagcgaatgggggtctcagattgccgcatccgcgtgtggtttcagaaccgcaggaatcgcagtggagaggaggggcatgcctcaaagaggtccatcagaggctccaggcggctagcctcgccacagctccaggaagagcttggatccaggccacagggtagaggcatgcgctcatctggcagaaggcctcgcactcgactcacctcgctacagctcaggatcctagggcaagcctttgagaggaacccacgaccaggctttgctaccagggaggagctggcgcgtgacacagggttgcccgaggacacgatccacatatggtttcaaaaccgaagagctcggcggcgccacaggaggggcaggcccacagctcaagatcaagacttgctggcgtcacaagggtcggatggggcccctgcaggtccggaaggcagagagcgtgaaggtgcccaggagaacttgttgccacaggaagaagcaggaagtacgggcatggatacctcgagccctagcgacttgccctccttctgcggagagtcccagcctttccaagtggcacagccccgtggagcaggccaacaagaggcccccactcgagcaggcaacgcaggctctctggaacccctccttgatcagctgctggatgaagtccaagtagaagagcctgctccagcccctctgaatttggatggagaccctggtggcagggtgcatgaaggttcccaggagagcttttggccacaggaagaagcaggaagtacaggcatggatacttctagccccagcgactcaaactccttctgcagagagtcccagccttcccaagtggcacagccctgtggagcgggccaagaagatgcccgcactcaagcagacagcacaggccctctggaactcctcctccttgatcaactgctggacgaagtccaaaaggaagagcatgtgccagtcccactggattggggtagaaatcctggcagcagggagcatgaaggttcccaggacagcttactgcccctggaggaagcagtaaattcgggcatggatacctcgatccctagcatctggccaaccttctgcagagaatcccagcctccccaagtggcacagccctctggaccaggccaagcacaggcccccactcaaggtgggaacacggaccccctggagctcttcctctatcaactgttggatgaagtccaagtagaagagcatgctccagcccctctgaattgggatgtagatcctggtggcagggtgcatgaaggttcgtgggagagcttttggccacaggaagaagcaggaagtacaggcctggatacttcaagccccagcgactcaaactccttcttcagagagtccaagccttcccaagtggcacagcgccgtggagcgggccaagaagatgcccgcactcaagcagacagcacaggccctctggaactcctcctctttgatcaactgctggacgaagtccaaaaggaagagcatgtgccagccccactggattggggtagaaatcctggcagcatggagcatgaaggttcccaggacagcttactgcccctggaggaagcagcaaattcgggcagggatacctcgatccctagcatctggccagccttctgcagaaaatcccagcctccccaagtggcacagccctctggaccaggccaagcacaggcccccattcaaggtgggaacacggaccccctggagctcttccttgatcaactgctgaccgaagtccaacttgaggagcaggggcctgcccctgtgaatgtggaggaaacatgggagcaaatggacacaacacctatctgcctctcacttcagaagaatatcagactcttctagatatgctctga

SEQ ID NO: 28 - RNA, 23 nt, synthetic construct
  source 1..23: mol_type = other RNA
tgttcttccgtgaaattctggct

(19) United States
(12) Patent Application Publication
(10) Pub. No.: US 2026/0053941 A1
(43) Pub. Date: Feb. 26, 2026

(54) MUSCLE TARGETING COMPLEXES AND FORMULATIONS FOR TREATING FACIOSCAPULOHUMERAL MUSCULAR DYSTROPHY

(71) Applicant: Dyne Therapeutics, Inc., Waltham, MA (US)

(72) Inventors: Timothy Weeden, Waltham, MA (US); Nelson Hsia, Waltham, MA (US); Stefano Zanotti, Waltham, MA (US); Oxana Beskrovnaya, Waltham, MA (US); Qifeng Qiu, Waltham, MA (US); Scott Hilderbrand, Waltham, MA (US); Jochen Deckert, Bayreuth (DE); Hans-Peter Vornlocher, Kulmbach (DE); Markus Hossbach, Kulmbach (DE); Kathrin Hultsch, Kulmbach (DE); Nicholas C. Yoder, Brookline, MA (US); Benjamin Vieira, Waltham, MA (US)

(73) Assignee: Dyne Therapeutics, Inc., Waltham, MA (US)

(21) Appl. No.: 19/382,888

(22) Filed: Nov. 7, 2025

Related U.S. Application Data
(63) Continuation of application No. PCT/US2024/061427, filed on Dec. 20, 2024.
(60) Provisional application No. 63/614,024, filed on Dec. 22, 2023.

(51) Int. Cl. A61K 47/68; A61P 21/00; C07K 16/28

(52) U.S. Cl. CPC A61K 47/6807; A61K 47/6849; A61K 47/6889; A61P 21/00; C07K 16/2881

(57) ABSTRACT

Aspects of the disclosure relate to complexes and other aspects relate to formulations (e.g., aqueous, lyophilized forms) comprising such complexes comprising an oligonucleotide (e.g., an RNAi oligonucleotide such as an siRNA, useful for targeting DUX4) covalently linked to an antibody (e.g., anti-TfR1 antibody).

### RELATED APPLICATIONS

This application claims the benefit under 35 U.S.C. § 119(e) to U.S. Provisional Application No. 63/614,024, filed Dec. 22, 2023, entitled “MUSCLE TARGETING COMPLEXES AND FORMULATIONS FOR TREATING FACIOSCAPULOHUMERAL MUSCULAR DYSTROPHY”, the contents of which is incorporated herein by reference in its entirety.

### FIELD OF THE INVENTION

The present application relates to targeting complexes for delivering oligonucleotide molecular payloads to cells, compositions (e.g., formulations) comprising such complexes, and uses thereof, particularly uses relating to treatment of disease.

### REFERENCE TO AN ELECTRONIC SEQUENCE LISTING

The contents of the electronic sequence listing (D082470086WO00-SEQ-CBD.xml; Size: 37,691 bytes; and Date of Creation: Dec. 20, 2024) is herein incorporated by reference in its entirety.

### BACKGROUND

Muscular dystrophies (MDs) are a group of diseases characterized by the progressive weakness and loss of muscle mass. These diseases are caused by mutations in genes which encode proteins needed for healthy muscle tissue. Facioscapulohumeral muscular dystrophy (FSHD) is a dominantly inherited type of MD which primarily affects muscles of the face, shoulder blades, and upper arms. Other symptoms of FSHD include abdominal muscle weakness, retinal abnormalities, hearing loss, and joint pain and inflammation. FSHD is the most prevalent of the nine types of MD affecting both adults and children, with a worldwide incidence of about 1 in 8,300 people. FSHD is caused by aberrant production of double homeobox 4 (DUX4), a protein that regulates gene expression. The DUX4 gene, which encodes the DUX4 protein, is located in the D4Z4 repeat region on chromosome 4 and is typically expressed in adult testes and thymus, and in 2-cell stage embryos, after which it is repressed by hypermethylation of the D4Z4 repeats which surround and compact the DUX4 gene. Two types of FSHD, Type 1 and Type 2 have been described. Type 1, which accounts for about 95% of cases, is associated with deletions of D4Z4 repeats in the subtelomeric region of chromosome 4. Unaffected individuals generally have more than 10 repeats arrayed in the subtelomeric region of chromosome 4, whereas the most common form of FSHD (FSHD1) is caused by a contraction of the array to fewer than 10 repeats, associated with decreased epigenetic repression and variegated expression of DUX4 in skeletal muscle. Two allelic variants of chromosome 4q (4qA and 4qB) exist in the most distal unit of the D4Z4 repeats. 4qA is in cis with a functional polyadenylation consensus site. Contractions on 4qA alleles are pathogenic because the DUX4 transcript is polyadenylated and stable. Type 2 FSHD, which accounts for about 5% of cases, is associated with mutations of the SMCHD1 gene on chromosome 18. Type 2 FSHD may also be associated with the DNMT3B gene or LRIF1 gene. Besides supportive care and treatments to address the symptoms of the disease, there are no effective therapies for FSHD.

### SUMMARY

According to some aspects, the present disclosure provides complexes and compositions (e.g., formulations) comprising such complexes that comprise an oligonucleotide covalently linked to an anti-transferrin receptor 1 (TfR1) antibody, wherein the anti-TfR1 antibody comprises: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16, wherein the oligonucleotide is an RNAi oligonucleotide targeting DUX4, and wherein the complexes are formulated with tris(hydroxymethyl)aminomethane and sucrose.

According to some aspects, the present disclosure provides compositions comprising complexes comprising a structure of formula (I): [R¹]ₙ₁—R², wherein each R¹ independently comprises a group of the formula (Ia):

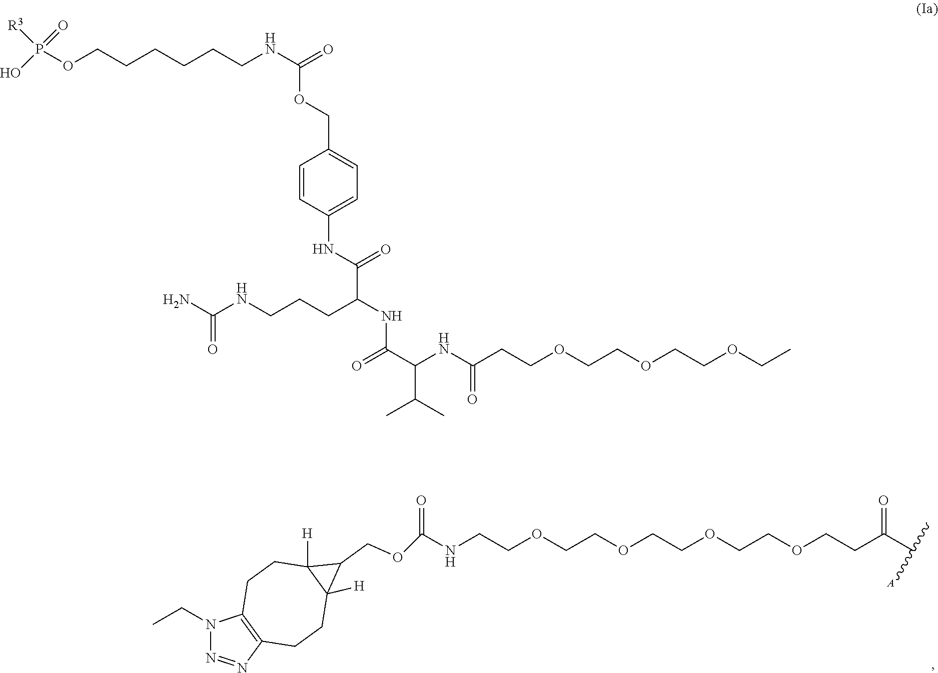

or a pharmaceutically acceptable salt thereof,
wherein
   R² comprises an antibody, and
   R³ comprises an RNAi oligonucleotide;
   wherein R¹ is covalently linked to R² at attachment point A; and
   wherein n1 is an integer representing the number of instances of R¹, wherein each instance of R¹ is covalently linked to a different amino acid residue of the antibody;
   wherein the complexes are formulated with tris(hydroxymethyl)aminomethane and sucrose.

In some embodiments, each different amino acid residue is a lysine.

In some embodiments, the antibody is an anti-TfR1 antibody.

In some embodiments, the antibody comprises: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16.

In some embodiments, the formulation is in a lyophilized form, a frozen solid form, or in an aqueous solution. In some embodiments, the formulation is in a lyophilized form. In some embodiments, the formulation is in a frozen solid form. In some embodiments, the formulation is in an aqueous solution.

In some embodiments, the formulation is in an aqueous solution and wherein the tris(hydroxymethyl)aminomethane is present in the aqueous solution at a concentration in the range of 5 mM to 50 mM.

In some embodiments, the formulation is in an aqueous solution and wherein the sucrose is present in the aqueous solution at a concentration in the range of 5% to 15% weight per volume (w/v %).

In some embodiments, the formulation is in an aqueous solution and wherein the aqueous solution has a pH in the range of 6.5 to 8.5.

In some embodiments, the formulation is in an aqueous solution and wherein the tris(hydroxymethyl)aminomethane is present in the aqueous solution at a concentration of 25 mM and/or the sucrose is present in the aqueous solution at a concentration of 10 w/v % and/or the aqueous solution is at a pH of 7.5.

In some embodiments, the antibody is a Fab fragment, a full-length IgG, a Fab′ fragment, a F(ab′)₂ fragment, an scFv, or an Fv.

In some embodiments, the antibody is a Fab fragment.

In some embodiments, the antibody comprises a heavy chain variable region (VH) comprising an amino acid sequence at least 85% identical to SEQ ID NO: 17; and/or wherein the antibody comprises a light chain variable region (VL) comprising an amino acid sequence at least 85% identical to SEQ ID NO: 18.

In some embodiments, the antibody comprises a VH comprising the amino acid sequence of SEQ ID NO: 17 and/or a VL comprising the amino acid sequence of SEQ ID NO: 18.

In some embodiments, the antibody comprises a heavy chain comprising an amino acid sequence at least 85% identical to SEQ ID NO: 19; and/or wherein the antibody comprises a light chain comprising an amino acid sequence at least 85% identical to SEQ ID NO: 20.

In some embodiments, the antibody comprises a heavy chain comprising the amino acid sequence of SEQ ID NO: 19 and/or a light chain comprising the amino acid sequence of SEQ ID NO: 20.

In some embodiments, the VH comprises an N-terminal pyroglutamate.

In some embodiments, the RNAi oligonucleotide comprises an antisense strand of 18-25 nucleosides in length.

In some embodiments, the antisense strand comprises a region of complementarity of at least 16 consecutive nucleosides in length to a nucleobase sequence as set forth in SEQ ID NO: 24.

In some embodiments, the region of complementarity is 22 consecutive nucleosides in length.

In some embodiments, the antisense strand comprises a region of complementarity of at least 16 consecutive nucleosides in length to a nucleobase sequence as set forth in SEQ ID NO: 28.

In some embodiments, the region of complementarity is 22 consecutive nucleosides in length.

In some embodiments, the RNAi oligonucleotide comprises an antisense strand comprising at least 15 consecutive nucleosides of a nucleobase sequence as set forth in SEQ ID NO: 22.

In some embodiments, the antisense strand comprises the nucleobase sequence of SEQ ID NO: 22.

In some embodiments, the RNAi oligonucleotide further comprises a sense strand comprising at least 15 consecutive nucleosides complementary to the antisense strand.

In some embodiments, the sense strand comprises at least 15 consecutive nucleosides of a nucleobase sequence as set forth in SEQ ID NO: 21.

In some embodiments, the sense strand comprises the nucleobase sequence of SEQ ID NO: 21.

In some embodiments, the RNAi oligonucleotide comprises one or more modified nucleosides.

In some embodiments, the RNAi oligonucleotide further comprises one or more phosphorothioate internucleoside linkages.

In some embodiments, the one or more modified nucleosides are 2′ modified nucleosides.

In some embodiments, wherein each 2′ modified nucleoside is 2′-O-methyl (2′-O-Me) or 2′-fluoro (2′-F).

In some embodiments, the antisense strand further comprises a 5′-(E)-vinylphosphonate.

In some embodiments, each R¹ comprises a group of the formula (Ib):

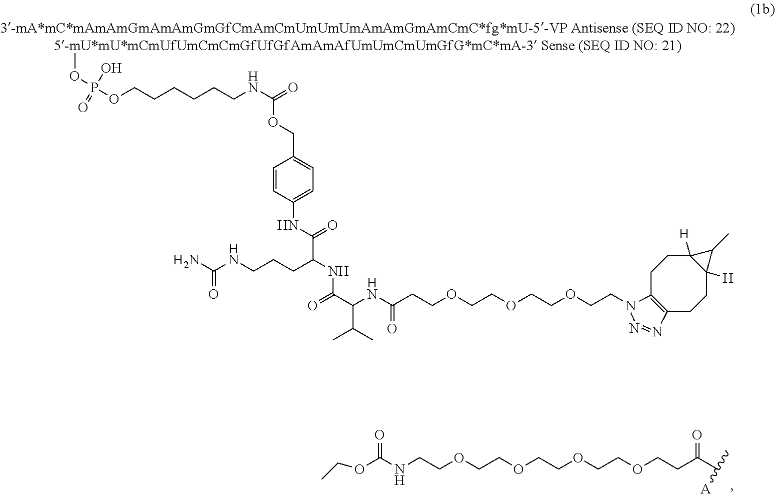

or a pharmaceutically acceptable salt thereof,
wherein: mA, mC, mG, and mU are 2′-O-methyl adenosine, 2′-O-methyl cytidine, 2′-O-methyl guanosine, and 2′-O-methyl uridine, respectively;
fA, fC, fG, and fU are 2′-fluoro adenosine, 2′-fluoro cytidine, 2′-fluoro guanosine, and 2′-fluoro uridine, respectively; * between two nucleosides represents a phosphorothioate linkage; the absence of “*” between two nucleosides represents a phosphodiester internucleoside linkage; and “VP” represents 5′-(E)-Vinylphosphonate, such that the RNAi oligonucleotide comprises an antisense strand comprising a nucleobase sequence of UGCCAGAAUUUCACGGAAGAACA (SEQ ID NO: 22) and a sense strand comprising a nucleobase sequence of UUCUUCCGUGAAAUUCUGGCA (SEQ ID NO: 21).

In some embodiments, each R¹ comprises a group of the formula (Ic), in which the antisense strand and the sense strand form a double stranded oligonucleotide:

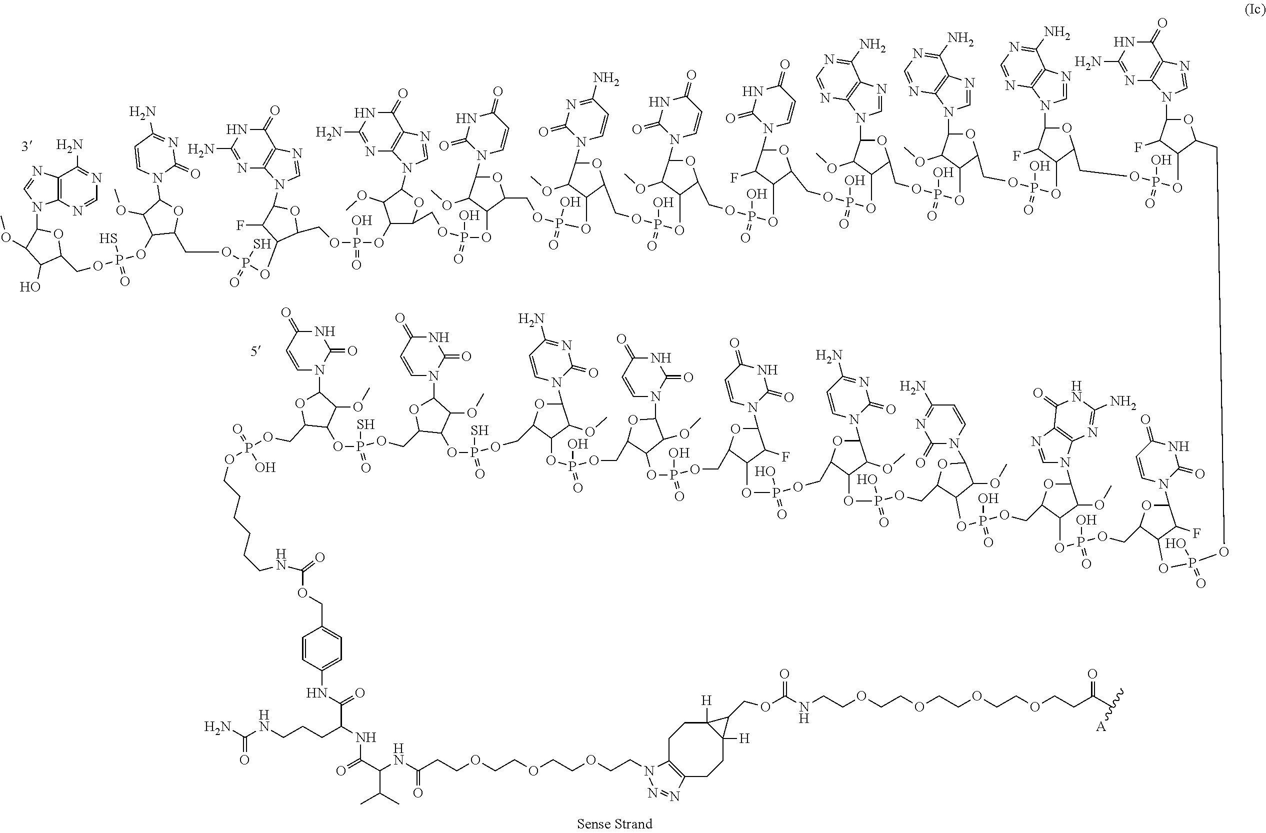

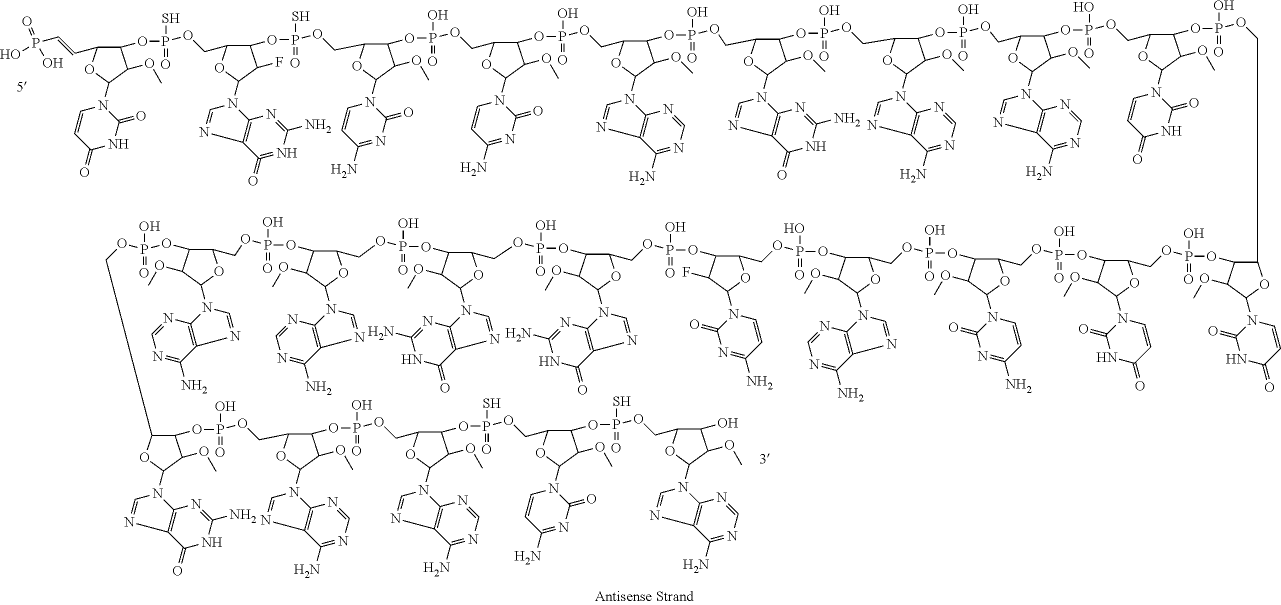

or a pharmaceutically acceptable salt thereof.

In some embodiments, the complexes are present in the composition at a concentration in the range of 10 mg/mL to 200 mg/mL.

In some embodiments, the compositions further comprise one or more antibodies that are not covalently linked to an oligonucleotide.

In some embodiments, the average value of n1 of complexes in the composition is in the range of 0.5 to 5.

In some embodiments, the average value of n1 of complexes in the composition is 1.

Further provided herein are methods of reducing DUX4 expression in a subject, the method comprising administering to the subject an effective amount of the compositions provided herein.

Further provided herein are methods of treating facioscapulohumeral muscular dystrophy (FSHD) in a subject, the method comprising administering to the subject an effective amount of the composition provided herein.

In some embodiments, the subject has aberrant production of DUX4 protein.

In some embodiments, the complexes reduce DUX4 expression in the subject.

In some embodiments, reducing DUX4 expression comprises reducing DUX4 protein and/or mRNA levels.

In some embodiments, the antibody comprises a VH that comprises an N-terminal pyroglutamate.

According to some aspects, the present disclosure provides complexes comprising a structure of formula (I): [R¹]ₙ₁—R², wherein each R¹ comprises a group of the formula (Ia):

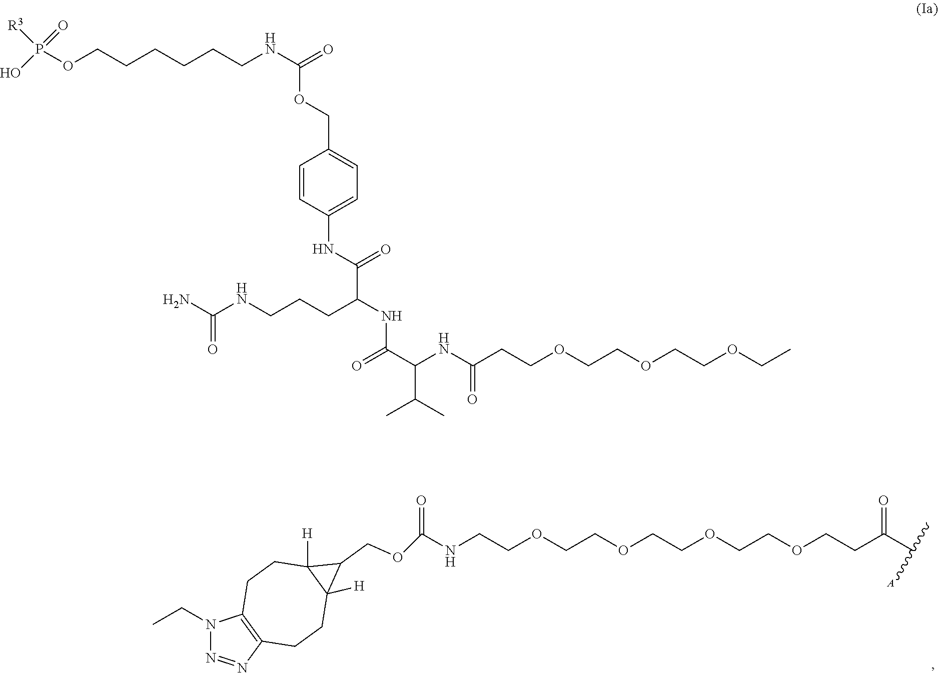

or a pharmaceutically acceptable salt thereof,
wherein R³ comprises an RNAi oligonucleotide comprising an antisense strand comprising a nucleobase sequence of SEQ ID NO: 22 and a structure (5′→3′) of VP-mU*fG*mCmCmAmGmAmAmUmUmUmCmAfCmGmGmAmAmGmAmA*mC*mA, and
   a sense strand comprising a nucleobase sequence of SEQ ID NO: 21 and a structure (5′→3′) of mU*mU*mCmUfUmCmCmGfUfGfAmAmAfUmUmCmUmGfG*mC*mA, wherein: mA, mC, mG, and mU are 2′-O-methyl adenosine, 2′-O-methyl cytidine, 2′-O-methyl guanosine, and 2′-O-methyl uridine, respectively; fA, fC, fG, and fU are 2′-fluoro adenosine, 2′-fluoro cytidine, 2′-fluoro guanosine, and 2′-fluoro uridine, respectively; * between two nucleosides represents a phosphorothioate linkage; the absence of “*” between two nucleosides represents a phosphodiester internucleoside linkage; and “VP” represents 5′-(E)-Vinylphosphonate;
   wherein R² comprises a Fab, and wherein the Fab comprises a CDR-H1, a CDR-H2, a CDR-H3, a CDR-L1, a CDR-L2, and a CDR-L3 selected from Table 2.

In some embodiments, the Fab comprises a VH comprising the amino acid sequence of SEQ ID NO: 17 and a VL comprising the amino acid sequence of SEQ ID NO: 18.

In some embodiments, the Fab comprises a VH comprising an N-terminal pyroglutamate.

In some embodiments, the Fab comprises a heavy chain comprising the amino acid sequence of SEQ ID NO: 19 and a light chain comprising the amino acid sequence of SEQ ID NO: 20; wherein R¹ is covalently linked to R² at attachment point A; and wherein n1 is an integer representing the number of instances of R¹, wherein each instance of R¹ is covalently linked to a different amino acid residue of the Fab.

In some embodiments, each different amino acid residue is a lysine.

According to some aspects, the present disclosure provides complexes comprising a structure of formula (I): [R¹]ₙ₁—R², wherein
each R¹ comprises a group of the formula (Ib):

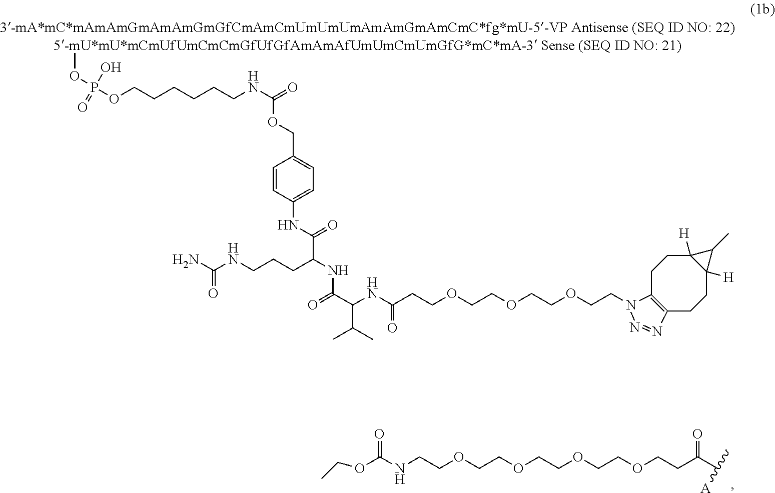

or a pharmaceutically acceptable salt thereof,
wherein: mA, mC, mG, and mU are 2′-O-methyl adenosine, 2′-O-methyl cytidine, 2′-O-methyl guanosine, and 2′-O-methyl uridine, respectively; fA, fC, fG, and fU are 2′-fluoro adenosine, 2′-fluoro cytidine, 2′-fluoro guanosine, and 2′-fluoro uridine, respectively; * between two nucleosides represents a phosphorothioate linkage; the absence of “*” between two nucleosides represents a phosphodiester internucleoside linkage; and “VP” represents 5′-(E)-Vinylphosphonate, such that the oligonucleotide is an RNAi oligonucleotide comprising an antisense strand comprising a nucleobase sequence of UGCCAGAAUUUCACGGAAGAACA (SEQ ID NO: 22) and a sense strand comprising a nucleobase sequence of UUCUUCCGUGAAAUUCUGGCA (SEQ ID NO: 21);
   wherein R² comprises a Fab, and wherein the Fab comprises a CDR-H1, a CDR-H2, a CDR-H3, a CDR-L1, a CDR-L2, and a CDR-L3 selected from Table 2.

In some embodiments, the Fab comprises a VH comprising the amino acid sequence of SEQ ID NO: 17 and a VL comprising the amino acid sequence of SEQ ID NO: 18.

In some embodiments, the Fab comprises a VH comprising an N-terminal pyroglutamate.

In some embodiments, the Fab comprises a heavy chain comprising the amino acid sequence of SEQ ID NO: 19 and a light chain comprising the amino acid sequence of SEQ ID NO: 20;
wherein R¹ is covalently linked to R² at attachment point A; and wherein n1 is an integer representing the number of instances of R¹, wherein each instance of R¹ is covalently linked to a different amino acid residue of the Fab.

In some embodiments, each different amino acid residue is a lysine.

According to some aspects, the present disclosure provides complexes comprising a structure of formula (I): [R¹]ₙ₁—R², wherein
R¹ comprises a group of the formula (Ic), in which the antisense strand and the sense strand form a double stranded oligonucleotide:

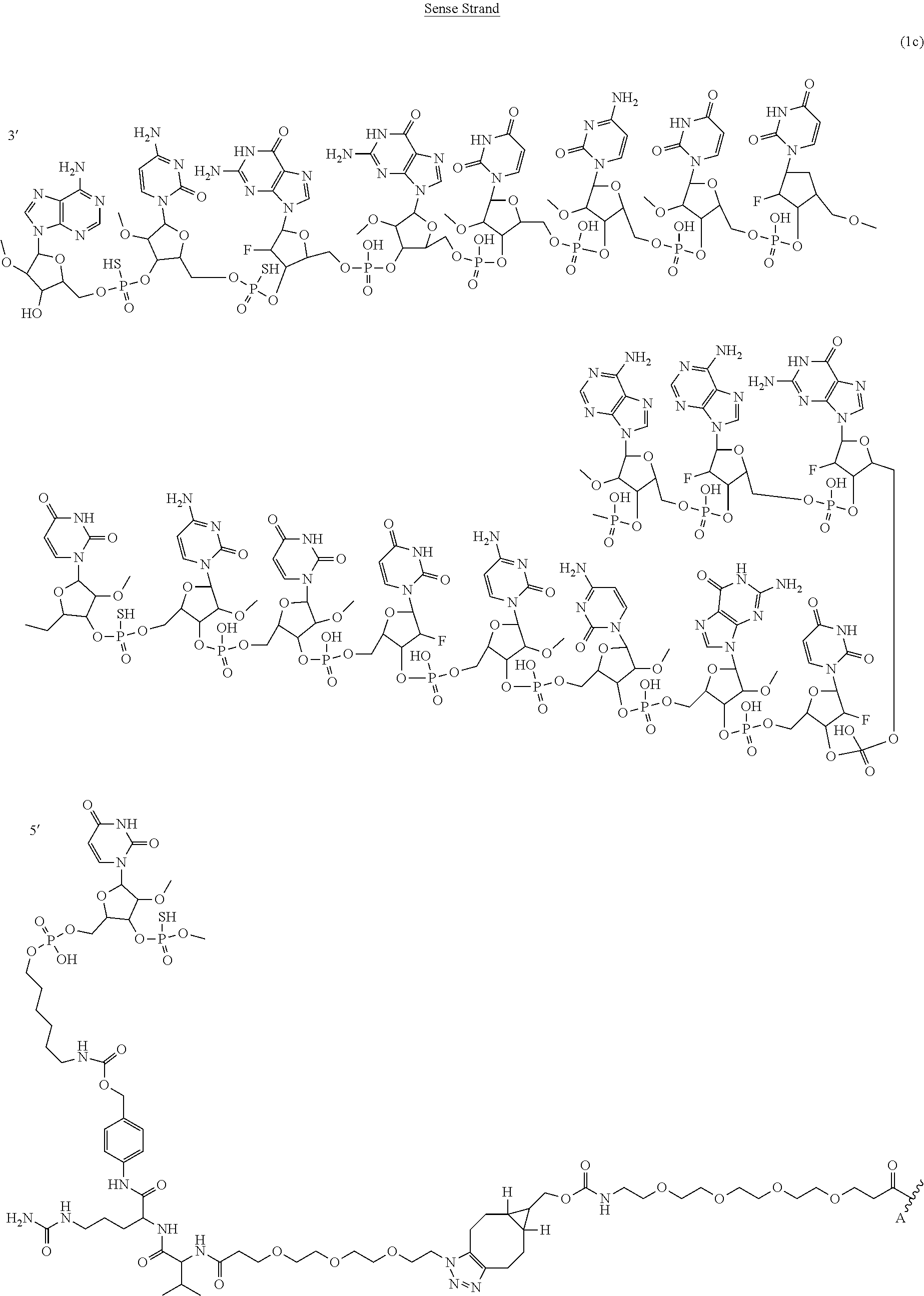

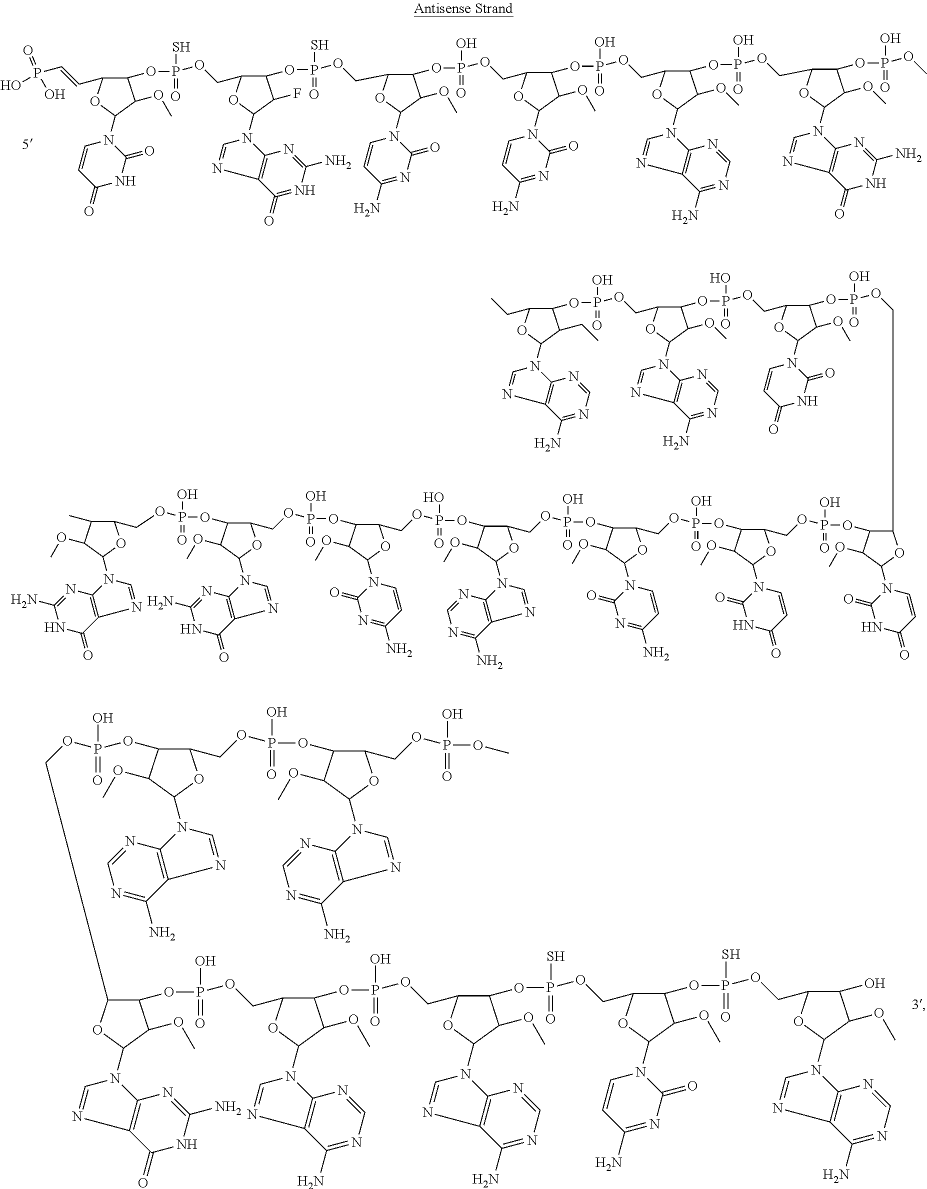

or a pharmaceutically acceptable salt thereof,
   wherein R² comprises a Fab comprising a CDR-H1, a CDR-H2, a CDR-H3, a CDR-L1, a CDR-L2, and a CDR-L3 selected from Table 2.

In some embodiments, the Fab comprises a VH comprising the amino acid sequence of SEQ ID NO: 17 and a VL comprising the amino acid sequence of SEQ ID NO: 18.

In some embodiments the Fab comprises a VH comprising an N-terminal pyroglutamate.

In some embodiments the Fab comprises a heavy chain comprising the amino acid sequence of SEQ ID NO: 19 and a light chain comprising the amino acid sequence of SEQ ID NO: 20; wherein R¹ is covalently linked to R² at attachment point A;
wherein n1 is an integer representing the number of instances of R¹, wherein each instance of R¹ is covalently linked to a different amino acid residue of the Fab.

In some embodiments, each different amino acid residue is a lysine.

According to some aspects, the present disclosure provides complexes comprising a structure of the formula (Id):

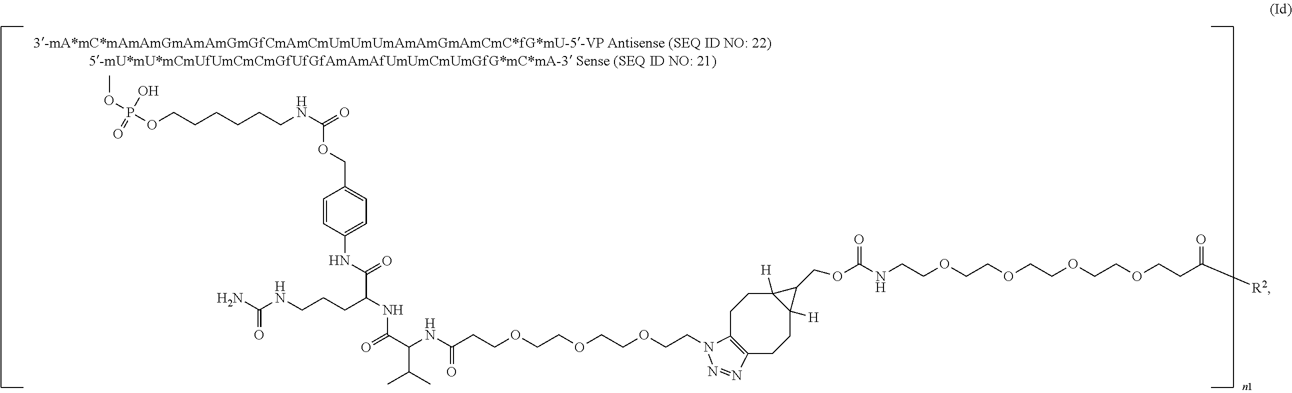

or a pharmaceutically acceptable salt thereof,
   wherein: mA, mC, mG, and mU are 2′-O-methyl adenosine, 2′-O-methyl cytidine, 2′-O-methyl guanosine, and 2′-O-methyl uridine, respectively;
   fA, fC, fG, and fU are 2′-fluoro adenosine, 2′-fluoro cytidine, 2′-fluoro guanosine, and 2′-fluoro uridine, respectively; * between two nucleosides represents a phosphorothioate linkage; the absence of “*” between two nucleosides represents a phosphodiester internucleoside linkage; and “VP” represents 5′-(E)-Vinylphosphonate, such that the oligonucleotide is an RNAi oligonucleotide comprising an antisense strand comprising a nucleobase sequence of UGCCAGAAUUUCACGGAAGAACA (SEQ ID NO: 22) and a sense strand comprising a nucleobase sequence of UUCUUCCGUGAAAUUCUGGCA (SEQ ID NO: 21;
   wherein R² comprises a Fab, and wherein the Fab comprises a CDR-H1, a CDR-H2, a CDR-H3, a CDR-L1, a CDR-L2, and a CDR-L3 selected from Table 2.

In some embodiments, the Fab comprises a VH comprising the amino acid sequence of SEQ ID NO: 17 and a VL comprising the amino acid sequence of SEQ ID NO: 18.

In some embodiments, the Fab comprises a VH comprising an N-terminal pyroglutamate.

In some embodiments, the Fab comprises a heavy chain comprising the amino acid sequence of SEQ ID NO: 19 and a light chain comprising the amino acid sequence of SEQ ID NO: 20;
wherein n1 is an integer representing the number of instances of the group enclosed by square brackets, wherein each instance of the group enclosed by square brackets is covalently linked to a different amino acid residue of the Fab.

In some embodiments, each different amino acid residue is a lysine.

According to some aspects, provided herein are formulations comprising a plurality of complexes of provided herein, and tris(hydroxymethyl)aminomethane at a concentration of 5 to 50 mM, and sucrose at a concentration of 2 w/v % to 15 w/v %, wherein the formulation is an aqueous solution and is at a pH of 6.5 to 8.5. In some embodiments, the plurality of complexes are at a concentration of 10 mg/mL to 200 mg/mL.

According to some aspects, provided herein are formulations comprising a plurality of complexes provided herein, and tris(hydroxymethyl)aminomethane at a concentration of 5 to 50 mM, and sucrose at a concentration of 3 w/v % to 10 w/v %, wherein the formulation is an aqueous solution and is at a pH of 6.5 to 8.5. In some embodiments, the plurality of complexes are at a concentration of 10 mg/mL to 200 mg/mL.

According to some aspects, provided herein are formulations comprising a plurality of complexes provided herein, and tris(hydroxymethyl)aminomethane at a concentration of 25 mM, and sucrose at a concentration of 10 w/v %, wherein the formulation is an aqueous solution and is at a pH of 7.5. In some embodiments, the plurality of complexes are at a concentration of 10 mg/mL to 200 mg/mL.

In some embodiments, the formulations further comprise one or more antibodies that are not covalently linked to an oligonucleotide.

In some embodiments, the average value of n1 of complexes in the formulation is in the range of 0.5 to 5.

In some embodiments, the average value of n1 of complexes in the formulation is 1.

According to some aspects, a lyophilized form of a formulation provided herein is disclosed.

According to some aspects, a product produced by a process comprising lyophilizing a formulation provided herein is disclosed.

According to some aspects, a frozen form of a formulation provided herein is disclosed.

According to some aspects, a product produced by a process comprising freezing a formulation provided herein is disclosed.

According to some aspects, a lyophilized cake comprising a plurality of complexes provided herein, tris(hydroxymethyl)aminomethane, and sucrose is disclosed.

### BRIEF DESCRIPTION OF THE DRAWINGS

FIG. 1 shows a composite score of the mRNA levels of three DUX4 transcriptome (D4T) markers (MBD3L2, TRIM43, and ZSCAN4) in FSHD patient-derived C6 myotubes, following treatment with siRNA-antibody complexes at a concentration of 10 nM, 100 nM, or 1000 nM. The siRNA-antibody complexes contained an anti-TfR1 antibody provided in Table 2 covalently linked to a DUX4-targeting siRNA. An exon 1 targeting siRNA was used as a positive control.

FIGS. 2A-2B show a composite score of the mRNA levels of three DUX4 mouse transcriptome (D4T) markers (Wfdc3, Sord, Serpinb6c) in mouse muscle quadriceps (FIG. 2A) and gastrocnemius (FIG. 2B) muscles relative to vehicle-treated controls. Tissues were collected 4 weeks after a single intravenous injection of DUX4-targeting siRNA complexes covalently linked to anti-TfR1 antibody provided in Table 2 at a dose of 10 mg/kg of siRNA.

FIGS. 3A-3C show heat stability of complexes of formula (Id), comprising an anti-TfR1 antibody provided in Table 2 covalently linked to a DUX4-targeting siRNA in a formulation containing 25 mM tris(hydroxymethyl)aminomethane, 10% (w/v) sucrose, at a pH of 7.5. The complex was incubated at room temperature for 4 weeks, and the samples were analyzed using analytical ultra-high performance liquid chromatography-size exclusion chromatography (UPLC-SEC) (FIG. 3A). The x-axis corresponds to the Week 0 trace only with the other timepoints offset for clarity. SEC monomer percentage results are shown in FIG. 3B. SEC peak area results are shown in FIG. 3C.

FIGS. 4A-4B show heat stability of complexes of formula (Id), comprising an anti-TfR1 antibody provided in Table 2 covalently linked to a DUX4-targeting siRNA in a formulation containing 25 mM tris(hydroxymethyl)aminomethane, 10% (w/v) sucrose, at a pH of 7.5. The complex was analyzed using UNcle stability screening platform (Unchained Labs) and specifically, by the UNcle Tₘ/T_{agg} method, using BCM (barycentric mean) and SLS473 (static light scattering) analyses. The complex was analyzed at two different time points: week 0 (FIG. 4A) and week 4 (FIG. 4B) after incubation at room temperature.

FIG. 5 shows heat stability of the complexes of formula (Id), comprising an anti-TfR1 antibody provided in Table 2 covalently linked to a DUX4-targeting siRNA in comparison with the unconjugated DUX4-targeting siRNA duplex. Differential scanning fluorimetry (DSF) was used to measure heat stability of the complexes.

FIG. 6 shows binding affinity of complexes of formula (Id), containing an anti-TfR1 antibody provided in Table 2 covalently linked to a DUX4-targeting siRNA in a formulation containing 25 mM tris(hydroxymethyl)aminomethane, 10% (w/v) sucrose, at a pH of 7.5. The complexes were incubated at room temperature for four weeks and the binding affinity was analyzed by enzyme-linked immunosorbent assay (ELISA).

FIGS. 7A-7C show binding affinity (FIG. 7A) and stability (FIG. 7B and FIG. 7C) of complexes of formula (Id), containing an anti-TfR1 antibody provided in Table 2 covalently linked to a DUX4-targeting siRNA in two additional formulations. Formulation a contains 25 mM Tris, 3% (w/v) sucrose, at a pH of 7.5. Formulation b contains 25 mM Tris, 6% (w/v) sucrose+50 mM NaCl, at a pH of 7.5.

### DETAILED DESCRIPTION OF INVENTION

According to some aspects, the present disclosure provides complexes and compositions (e.g., formulations) comprising such complexes. In some embodiments, the complexes are formulated with tris(hydroxymethyl)aminomethane and sucrose. In some embodiments, the complexes are formulated as aqueous or lyophilized (e.g., lyophilized powder) forms. In some embodiments, a complex comprises an oligonucleotide (e.g., an RNAi oligonucleotide) covalently linked to an antibody. In some embodiments, a complex comprises a muscle-targeting complex comprising an oligonucleotide (e.g., an RNAi oligonucleotide) covalently linked to an anti-transferrin receptor 1 (TfR1) antibody. In some embodiments, a complex comprises a muscle-targeting complex comprising an oligonucleotide (e.g., an RNAi oligonucleotide) covalently linked to the anti-transferrin receptor 1 (TfR1) antibody provided in Table 2. In some embodiments, a complex comprises a muscle-targeting complex comprising an oligonucleotide (e.g., an RNAi oligonucleotide) targeting DUX4 covalently linked to the anti-transferrin receptor 1 (TfR1) antibody provided in Table 2. Also provided are methods of using the complexes and compositions (e.g., formulations) described herein for treating a subject having facioscapulohumeral muscular dystrophy (FSHD) and/or methods of reducing the expression or activity of DUX4 (e.g., DUX4 protein and/or mRNA) in a cell.

Further aspects of the disclosure, including a description of defined terms, are provided below.

### Definitions

Administering: As used herein, the terms “administering” or “administration” means to provide a complex to a subject in a manner that is physiologically and/or (e.g., and) pharmacologically useful (e.g., to treat a condition in the subject).

Approximately: As used herein, the term “approximately” or “about,” as applied to one or more values of interest, refers to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Antibody: As used herein, the term “antibody” refers to a polypeptide that includes at least one immunoglobulin variable domain or at least one antigenic determinant, e.g., paratope that specifically binds to an antigen. In some embodiments, an antibody is a full-length antibody. In some embodiments, an antibody is a chimeric antibody. In some embodiments, an antibody is a humanized antibody. However, in some embodiments, an antibody is a Fab fragment, a Fab′ fragment, a F(ab′)₂ fragment, a Fv fragment or a scFv fragment. In some embodiments, an antibody is a nanobody derived from a camelid antibody or a nanobody derived from shark antibody. In some embodiments, an antibody is a diabody. In some embodiments, an antibody comprises a framework having a human germline sequence. In another embodiment, an antibody comprises a heavy chain constant domain selected from the group consisting of IgG, IgG1, IgG2, IgG2A, IgG2B, IgG2C, IgG3, IgG4, IgA1, IgA2, IgD, IgM, and IgE constant domains. In some embodiments, an antibody comprises a heavy (H) chain variable region (abbreviated herein as VH), and/or (e.g., and) a light (L) chain variable region (abbreviated herein as VL). In some embodiments, an antibody comprises a constant domain, e.g., an Fc region. An immunoglobulin constant domain refers to a heavy or light chain constant domain. Human IgG heavy chain and light chain constant domain amino acid sequences and their functional variations are known. With respect to the heavy chain, in some embodiments, the heavy chain of an antibody described herein can be an alpha (α), delta (Δ), epsilon (ε), gamma (γ) or mu (μ) heavy chain. In some embodiments, the heavy chain of an antibody described herein can comprise a human alpha (α), delta (Δ), epsilon (ε), gamma (γ) or mu (μ) heavy chain. In a particular embodiment, an antibody described herein comprises a human gamma 1 CH1, CH2, and/or (e.g., and) CH3 domain. In some embodiments, the amino acid sequence of the VH domain comprises the amino acid sequence of a human gamma (γ) heavy chain constant region, such as any known in the art. Non-limiting examples of human constant region sequences have been described in the art, e.g., see U.S. Pat. No. 5,693,780 and Kabat E A et al., (1991) supra. In some embodiments, the VH domain comprises an amino acid sequence that is at least 70%, 75%, 80%, 85%, 90%, 95%, 98%, or at least 99% identical to any of the variable chain constant regions provided herein. In some embodiments, an antibody is modified, e.g., modified via glycosylation, phosphorylation, sumoylation, and/or (e.g., and) methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or (e.g., and) phosphoglycosylation. In some embodiments, the one or more sugar or carbohydrate molecule are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, an antibody is a construct that comprises a polypeptide comprising one or more antigen binding fragments of the disclosure linked to a linker polypeptide or an immunoglobulin constant domain. Linker polypeptides comprise two or more amino acid residues joined by peptide bonds and are used to link one or more antigen binding portions. Examples of linker polypeptides have been reported (see e.g., Holliger, P., et al. (1993) Proc. Natl. Acad. Sci. USA 90:64446448; Poljak, R. J., et al. (1994) Structure 2:1121-1123). Still further, an antibody may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058).

CDR: As used herein, the term “CDR” refers to the complementarity determining region within antibody variable sequences. A typical antibody molecule comprises a heavy chain variable region (VH) and a light chain variable region (VL), which are usually involved in antigen binding. The VH and VL regions can be further subdivided into regions of hypervariability, also known as “complementarity determining regions” (“CDR”), interspersed with regions that are more conserved, which are known as “framework regions” (“FR”). Each VH and VL is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the IMGT definition, the Chothia definition, the AbM definition, and/or (e.g., and) the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242; IMGT®, the international ImMunoGeneTics information System® http://www.imgt.org, Lefranc, M.-P. et al., Nucleic Acids Res., 27:209-212 (1999); Ruiz, M. et al., Nucleic Acids Res., 28:219-221 (2000); Lefranc, M.-P., Nucleic Acids Res., 29:207-209 (2001); Lefranc, M.-P., Nucleic Acids Res., 31:307-310 (2003); Lefranc, M.-P. et al., In Silico Biol., 5, 0006 (2004) [Epub], 5:45-60 (2005); Lefranc, M.-P. et al., Nucleic Acids Res., 33:D593-597 (2005); Lefranc, M.-P. et al., Nucleic Acids Res., 37:D1006-1012 (2009); Lefranc, M.-P. et al., Nucleic Acids Res., 43:D413-422 (2015); Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs. As used herein, a CDR may refer to the CDR defined by any method known in the art. Two antibodies having the same CDR means that the two antibodies have the same amino acid sequence of that CDR as determined by the same method, for example, the IMGT definition.

There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Sub-portions of CDRs may be designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems. Examples of CDR definition systems are provided in Table 1.

TABLE 1
CDR Definitions

| | IMGT¹ | Kabat² | Chothia³ |
|---|---|---|---|
| CDR-H1 | 27-38 | 31-35 | 26-32 |
| CDR-H2 | 56-65 | 50-65 | 53-55 |
| CDR-H3 | 105-116/117 | 95-102 | 96-101 |
| CDR-L1 | 27-38 | 24-34 | 26-32 |
| CDR-L2 | 56-65 | 50-56 | 50-52 |
| CDR-L3 | 105-116/117 | 89-97 | 91-96 |

¹IGMT ®, the international ImMunoGeneTics information system ®, imgt.org, Lefranc, M.-P. et al., Nucleic Acids Res., 27: 209-212 (1999)
²Kabat et al. (1991) Sequences of Proteins of Immunological Interest, Fifth Edition, U.S. Department of Health and Human Services, NIH Publication No. 91-3242
³Chothia et al., J. Mol. Biol. 196: 901-917 (1987))

Chimeric antibody: The term “chimeric antibody” refers to antibodies which comprise heavy and light chain variable region sequences from one species and constant region sequences from another species, such as antibodies having murine heavy and light chain variable regions linked to human constant regions.

Complementary: As used herein, the term “complementary” refers to the capacity for precise pairing between two nucleotides or nucleosides or two sets of nucleotides or nucleosides. In particular, complementary is a term that characterizes an extent of hydrogen bond pairing that brings about binding between two nucleotides or nucleosides or two sets of nucleotides or nucleosides. For example, if a base at one position of an oligonucleotide is capable of hydrogen bonding with a base at the corresponding position of a target nucleic acid (e.g., an mRNA), then the bases are considered to be complementary to each other at that position. Base pairings may include both canonical Watson-Crick base pairing and non-Watson-Crick base pairing (e.g., Wobble base pairing and Hoogsteen base pairing). For example, in some embodiments, for complementary base pairings, adenosine-type bases (A) are complementary to thymidine-type bases (T) or uracil-type bases (U), that cytosine-type bases (C) are complementary to guanosine-type bases (G), and that universal bases such as 3-nitropyrrole or 5-nitroindole can hybridize to and are considered complementary to any A, C, U, or T. Inosine (I) has also been considered in the art to be a universal base and is considered complementary to any A, C, U or T.

Covalently linked: As used herein, the term “covalently linked” refers to a characteristic of two or more molecules being linked together via at least one covalent bond. In some embodiments, two molecules can be covalently linked together by a single bond, e.g., a disulfide bond or disulfide bridge, that serves as a linker between the molecules. However, in some embodiments, two or more molecules can be covalently linked together via a molecule that serves as a linker that joins the two or more molecules together through multiple covalent bonds. In some embodiments, a linker may be a cleavable linker. However, in some embodiments, a linker may be a non-cleavable linker.

DUX4: As used herein, the term “DUX4” refers to a gene that encodes double homeobox 4, a protein which is generally expressed during fetal development and in the testes of adult males. In some embodiments, DUX4 may be a human (Gene ID: 100288687), non-human primate (e.g., Gene ID: 750891, Gene ID: 100405864), or rodent gene (e.g., Gene ID: 306226). In humans, expression of the DUX4 gene outside of fetal development and the testes is associated with facioscapulohumeral muscular dystrophy. In addition, multiple human transcript variants (e.g., as annotated under GenBank RefSeq Accession Numbers: NM_001293798.2, NM_001306068.3, NM_001363820.1) have been characterized that encode different protein isoforms.

Facioscapulohumeral muscular dystrophy (FSHD): As used herein, the term “facioscapulohumeral muscular dystrophy (FSHD)” refers to a genetic disease caused by mutations in the DUX4 gene, SMCHD1 gene, DNMT3B gene, or LRIF1 gene that is characterized by muscle mass loss and muscle atrophy, primarily in the muscles of the face, shoulder blades, and upper arms. Two types of the disease, Type 1 and Type 2, have been described. Type 1 is associated with deletions in D4Z4 repeat regions on chromosome 4 which contains the DUX4 gene. In some embodiments, Type 1 is associated with deletions in D4Z4 repeat regions on chromosome 4 allelic variant 4qA which contains the DUX4 gene. Type 2 is associated with mutations in the SMCHD1 gene, DNMT3B gene, or LRIF1 gene (see, e.g. Jia et al., “Facioscapulohumeral muscular dystrophy type 2: an update on the clinical, genetic, and molecular findings” Neuromuscul Disord. (2021), 31(11): 1101-1112. Both Type 1 and Type 2 FSHD are characterized by aberrant production of the DUX4 protein after fetal development outside of the testes. Facioscapulohumeral dystrophy, the genetic basis for the disease, and related symptoms are described in the art (see, e.g. Campbell, A. E., et al., “Facioscapulohumeral dystrophy: Activating an early embryonic transcriptional program in human skeletal muscle” Human Mol Genet. (2018); and Tawil, R. “Facioscapulohumeral muscular dystrophy” Handbook Clin. Neurol. (2018), 148: 541-548.) FSHD Type 1 is associated with Online Mendelian Inheritance in Man (OMIM) Entry #158900. FSHD Type 2 is associated with OMIM Entry #158901.

Framework: As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region. Human heavy chain and light chain acceptor sequences are known in the art. In one embodiment, the acceptor sequences known in the art may be used in the antibodies disclosed herein.

Human antibody: The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

Humanized antibody: The term “humanized antibody” refers to antibodies which comprise heavy and light chain variable region sequences from a non-human species (e.g., a mouse) but in which at least a portion of the VH and/or (e.g., and) VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding nonhuman CDR sequences. In one embodiment, humanized anti-transferrin receptor antibodies and antigen binding portions are provided. Such antibodies may be generated by obtaining murine anti-transferrin receptor monoclonal antibodies using traditional hybridoma technology followed by humanization using in vitro genetic engineering, such as those disclosed in Kasaian et al PCT publication No. WO 2005/123126 A2.

Internalizing cell surface receptor: As used herein, the term, “internalizing cell surface receptor” refers to a cell surface receptor that is internalized by cells, e.g., upon external stimulation, e.g., ligand binding to the receptor. In some embodiments, an internalizing cell surface receptor is internalized by endocytosis. In some embodiments, an internalizing cell surface receptor is internalized by clathrin-mediated endocytosis. However, in some embodiments, an internalizing cell surface receptor is internalized by a clathrin-independent pathway, such as, for example, phagocytosis, macropinocytosis, caveolae- and raft-mediated uptake or constitutive clathrin-independent endocytosis. In some embodiments, the internalizing cell surface receptor comprises an intracellular domain, a transmembrane domain, and/or (e.g., and) an extracellular domain, which may optionally further comprise a ligand-binding domain. In some embodiments, a cell surface receptor becomes internalized by a cell after ligand binding. In some embodiments, a ligand may be a muscle-targeting agent or a muscle-targeting antibody. In some embodiments, an internalizing cell surface receptor is a transferrin receptor.

Kabat numbering: The terms “Kabat numbering”, “Kabat definitions and “Kabat labeling” are used interchangeably herein. These terms, which are recognized in the art, refer to a system of numbering amino acid residues which are more variable (i.e. hypervariable) than other amino acid residues in the heavy and light chain variable regions of an antibody, or an antigen binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

Molecular payload: As used herein, the term “molecular payload” refers to a molecule or species that functions to modulate a biological outcome. In some embodiments, a molecular payload is linked to, or otherwise associated with a muscle-targeting agent. In some embodiments, the molecular payload is a small molecule, a protein, a peptide, a nucleic acid, or an oligonucleotide. In some embodiments, the molecular payload functions to modulate the transcription of a DNA sequence, to modulate the expression of a protein, or to modulate the activity of a protein. In some embodiments, the molecular payload is an oligonucleotide that comprises a strand having a region of complementarity to a target gene.

Oligonucleotide: As used herein, the term “oligonucleotide” refers to an oligomeric nucleic acid compound of up to 200 nucleotides in length. Examples of oligonucleotides include, but are not limited to, RNAi oligonucleotides (e.g., siRNAs, shRNAs), microRNAs, gapmers, mixmers, phosphorodiamidate morpholinos, peptide nucleic acids, aptamers, guide nucleic acids (e.g., Cas9 guide RNAs), etc. Oligonucleotides may be single-stranded or double-stranded. In some embodiments, an oligonucleotide may comprise one or more modified nucleosides (e.g., 2′-O-methyl sugar modifications, purine or pyrimidine modifications). In some embodiments, an oligonucleotide may comprise one or more modified internucleoside linkage. In some embodiments, an oligonucleotide may comprise one or more phosphorothioate linkages, which may be in the Rp or Sp stereochemical conformation.

Pharmaceutically acceptable salt: As used herein, the term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions, such as halide, hydroxide, carboxylate, sulfate, phosphate, nitrate, lower alkyl sulfonate, and aryl sulfonate.

Region of complementarity: As used herein, the term “region of complementarity” refers to a nucleotide sequence, e.g., of an oligonucleotide, that is sufficiently complementary to a cognate nucleotide sequence, e.g., of a target nucleic acid, such that the two nucleotide sequences are capable of annealing to one another under physiological conditions (e.g., in a cell). In some embodiments, a region of complementarity is fully complementary to a cognate nucleotide sequence of target nucleic acid. However, in some embodiments, a region of complementarity is partially complementary to a cognate nucleotide sequence of target nucleic acid (e.g., at least 80%, 90%, 95% or 99% complementarity). In some embodiments, a region of complementarity contains 1, 2, 3, or 4 mismatches compared with a cognate nucleotide sequence of a target nucleic acid.

Specifically binds: As used herein, the term “specifically binds” refers to the ability of a molecule to bind to a binding partner with a degree of affinity or avidity that enables the molecule to be used to distinguish the binding partner from an appropriate control in a binding assay or other binding context. With respect to an antibody, the term, “specifically binds”, refers to the ability of the antibody to bind to a specific antigen with a degree of affinity or avidity, compared with an appropriate reference antigen or antigens, that enables the antibody to be used to distinguish the specific antigen from others, e.g., to an extent that permits preferential targeting to certain cells, e.g., muscle cells, through binding to the antigen, as described herein. In some embodiments, an antibody specifically binds to a target if the antibody has a K_{D} for binding the target of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. In some embodiments, an antibody specifically binds to the transferrin receptor, e.g., an epitope of the apical domain of transferrin receptor.

Subject: As used herein, the term “subject” refers to a mammal. In some embodiments, a subject is non-human primate, or rodent. In some embodiments, a subject is a human. In some embodiments, a subject is a patient, e.g., a human patient that has or is suspected of having a disease. In some embodiments, the subject is a human patient who has or is suspected of having FSHD.

Transferrin receptor: As used herein, the term, “transferrin receptor” (also known as TFRC, CD71, p90, TFR, or TFR1) refers to an internalizing cell surface receptor that binds transferrin to facilitate iron uptake by endocytosis. In some embodiments, a transferrin receptor may be of human (NCBI Gene ID 7037), non-human primate (e.g., NCBI Gene ID 711568 or NCBI Gene ID 102136007), or rodent (e.g., NCBI Gene ID 22042) origin. In addition, multiple human transcript variants have been characterized that encoded different isoforms of the receptor (e.g., as annotated under GenBank RefSeq Accession Numbers: NP_001121620.1, NP_003225.2, NP_001300894.1, and NP_001300895.1).

2′-modified nucleoside: As used herein, the terms “2′-modified nucleoside” and “2′-modified ribonucleoside” are used interchangeably and refer to a nucleoside having a sugar moiety modified at the 2′ position. In some embodiments, the 2′-modified nucleoside is a 2′→4′ bicyclic nucleoside, where the 2′ and 4′ positions of the sugar are bridged (e.g., via a methylene, an ethylene, or a (S)-constrained ethyl bridge). In some embodiments, the 2′-modified nucleoside is a non-bicyclic 2′-modified nucleoside, e.g., where the 2′ position of the sugar moiety is substituted. Non-limiting examples of 2′-modified nucleosides include: 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-O—N-methylacetamido (2′-O-NMA), locked nucleic acid (LNA, methylene-bridged nucleic acid), ethylene-bridged nucleic acid (ENA), and (S)-constrained ethyl-bridged nucleic acid (cEt). In some embodiments, the 2′-modified nucleosides described herein are high-affinity modified nucleosides and oligonucleotides comprising the 2′-modified nucleosides have increased affinity to a target sequences, relative to an unmodified oligonucleotide. Examples of structures of 2′-modified nucleosides are provided below:

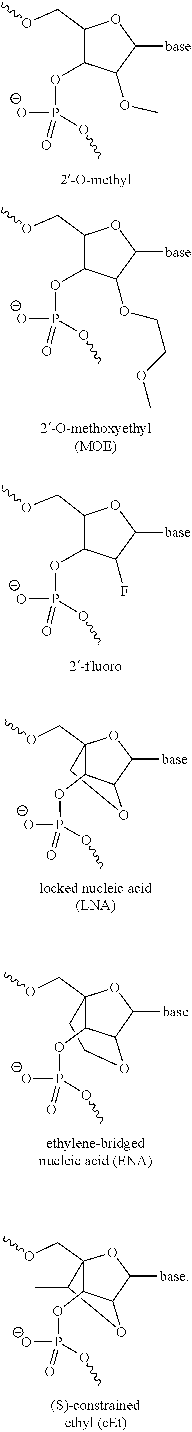

These examples are shown with phosphate groups, but any internucleoside linkages are contemplated between 2′-modified nucleosides. In certain embodiments, the internucleoside phosphate groups are as depicted, i.e., deprotonated in salt form. In certain embodiments, the internucleoside phosphate groups are protonated. The extent to which the phosphate groups are protonated or deprotonated will vary depending on the chemical environment of the nucleosides (e.g., pH, presence of basic media, etc.).

Ranges: All ranges provided in the present disclosure are inclusive of the end points.

### Complexes

Provided herein are complexes that comprise a targeting agent, e.g., an antibody, covalently linked to an oligonucleotide. In some embodiments, a complex comprises a muscle-targeting antibody (e.g., an anti-TfR1 antibody) covalently linked to one or more oligonucleotides. In some embodiments, the oligonucleotide is an RNAi oligonucleotide (e.g., siRNA) that targets a DUX4 RNA to reduce expression or activity of DUX4 (e.g., to reduce DUX4 protein and/or mRNA levels).

Complexes described herein generally comprise a linker that covalently links an antibody (e.g., an anti-TfR1 antibody) described herein to an oligonucleotide (e.g., an RNAi oligonucleotide). A linker comprises at least one covalent bond.

In some embodiments, complexes described herein comprise a structure of formula (I): [R₁]ₙ₁—R², in which each R¹ independently comprises a compound comprising an oligonucleotide (e.g., an RNAi oligonucleotide) and R² comprises an antibody (e.g., an anti-TfR1 antibody), and wherein in each complex n1 is independently an integer (e.g., one or greater) representing the number of instances of R¹ in each complex. In some embodiments, each R¹ independently comprises a group comprising an oligonucleotide. In some embodiments, each R¹ independently comprises a group that comprises additional elements in addition to an oligonucleotide. In some embodiments, R² comprises an antibody (e.g., an anti-TfR1 antibody) comprising a heavy chain comprising a heavy chain variable region (VH) and a heavy chain constant region, and a light chain comprising a light chain variable region (VL) and a light chain constant region. In some embodiments, each R¹ of a complex is independently covalently linked to a different amino acid residue (e.g., lysine or cysteine) of R².

In some embodiments, in each complex n1 is independently an integer (e.g., one or greater). In some embodiments, the antibody comprises a sequence as set forth in Table 2. For example, in some embodiments, the antibody comprises a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprises a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, the antibody comprises a heavy chain variable region (VH) comprising an amino acid sequence at least 85% (e.g., at least 95%) identical to SEQ ID NO: 17 and/or comprises a light chain variable region (VL) comprising an amino acid sequence at least 85% (e.g., at least 95%) identical to SEQ ID NO: 18. In some embodiments, the antibody comprises a VH comprising the amino acid sequence of SEQ ID NO: 17 and/or comprises a VL comprising the amino acid sequence of SEQ ID NO: 18. In some embodiments, the antibody comprises a heavy chain comprising an amino acid sequence at least 85% (e.g., at least 95%) identical to SEQ ID NO: 19 and/or comprises a light chain comprising an amino acid sequence at least 85% (e.g., at least 95%) identical to SEQ ID NO: 20. In some embodiments, the antibody comprises a heavy chain comprising the amino acid sequence of SEQ ID NO: 19 and/or comprises a light chain comprising the amino acid sequence of SEQ ID NO: 20. In some embodiments, the antibody is a Fab fragment, a full-length IgG, a Fab′ fragment, a F(ab′)₂ fragment, an scFv, or an Fv. In some embodiments, the antibody is a Fab fragment.

In some embodiments, the value of n1 of each or any complex (e.g., any complex in any of the compositions or formulations disclosed herein) is an integer up to the number of amino acid residues in the antibody to which conjugation is desired or targeted (e.g., the number of lysine residues). In some embodiments, in each complex the value of n1 is independently selected from 1, 2, 3, 4, 5, 6, 7, 8, 9, 10, 11, 12, 13, 14, 15, 16, 17, 18, 19, 20, 21, 22, 23, 24, 25, 26, and 27. In some embodiments, in each complex the value of n1 is independently selected from 1, 2, 3, 4, 5, 6, 7, 8, 9, 10, 11, 12, 13, 14, 15, 16, 17, 18, 19, 20, 21, 22, 23, 24, 25 and 26. In some embodiments, in each complex the value of n1 is 1. In some embodiments, in each complex the value of n1 is independently in the range of 1-27, 1-26, 1-10, 1-5, or 1-3. In some embodiments, the average value of n1 of complexes of the composition is in the range of 1 to 5 (e.g., 1-5, 1-4, 1-3, 3-5, or 1-2). In some embodiments, compositions described herein comprise complexes that comprise a structure of formula (I): [R¹]ₙ₁—R², wherein n1 is 0. In some embodiments, the average value of n1 of complexes of the composition is in the range of 0.5 to 5 (e.g., 0.5-5, 1-5, 1-4, 1-3, 3-5, 0.5-4, 0.5-3, 0.5-2, 0.5-1.5, 0.5-1, 0.7-1.5, 1-1.6, 1-1.5, 1-1.4, 1-1.3, 1-1.2, 1.1-1.5, 0.8-2, 0.8-1.5, 0.8-1.3, 0.8-1.2, 0.8-1.1, 0.9-3, 0.9-2, 0.9-1.8, 0.9-1.6, 0.9-1.5, 0.9-1.4, 0.9-1.3, or 0.9-1.2). In some embodiments, the average value of n1 of complexes of the composition is 1. In some embodiments, in each complex type n1 is independently an integer of one or greater representing the number of instances of R¹ in each complex of the complex type, and in which the different complex types of the composition are characterized by having different n1 values (e.g., n1 values in the range of 1-27, 1-26, 1-25, 1-20, 1-15, 1-10, 1-5, or 1-3).

In some embodiments, compositions are provided (e.g., formulations comprising tris(hydroxymethyl)aminomethane and/or sucrose, as described herein) that comprise a plurality of different complexes. In some embodiments, the plurality of different complexes comprise a common targeting agent (e.g. an antibody) and a common oligonucleotide (e.g., an RNAi oligonucleotide, such as a DUX4-targeting RNAi oligonucleotide). In such embodiments, different complex types are characterized by having different numbers of oligonucleotides covalently linked to an antibody. For example, in some embodiments, compositions are provided that comprise a plurality of complexes comprising a structure of formula (I): [R¹]ₙ₁—R², in which each R¹ independently comprises a compound comprising an oligonucleotide (e.g., a DUX4-targeting RNAi oligonucleotide) and R² comprises an antibody (e.g., anti-TfR1 antibody), and in which n1 is an integer representing the number of instances of R¹ in a complex, and in which different complexes of the composition may have different n1 values (e.g., n1 values in the range of 1-27, 1-26, 1-10, 1-5, or 1-3). In some embodiments, in complexes of a composition n1 is independently an integer. In some embodiments, in complexes of a composition n1 is 1. In some embodiments, the average value of n1 of complexes of the composition is in the range of 0.5 to 5 (e.g., 0.5-5, 1-5, 1-4, 1-3, 3-5, 0.5-4, 0.5-3, 0.5-2, 0.5-1.5, 0.5-1, 0.7-1.5, 1-1.6, 1-1.5, 1-1.4, 1-1.3, 1-1.2, 1.1-1.5, 0.8-2, 0.8-1.5, 0.8-1.3, 0.8-1.2, 0.8-1.1, 0.9-3, 0.9-2, 0.9-1.8, 0.9-1.6, 0.9-1.5, 0.9-1.4, 0.9-1.3, or 0.9-1.2). In some embodiments, the average value of n1 of complexes of the composition is 1. In some embodiments, compositions described herein comprise complexes in which n1 is 0.

In some embodiments, a composition described herein comprises an antibody that is not conjugated to an oligonucleotide (e.g., in trace amounts) and an antibody conjugated to one or more oligonucleotides. In some embodiments, the antibody that is not conjugated to an oligonucleotide may be referred to as a compound comprising a structure of formula (I): [R¹]ₙ₁—R², for which n1 is zero. Accordingly, in some embodiments, a composition for administration to a subject in the methods described herein comprises compounds (e.g., complexes) comprising a structure of formula (I): [R¹]ₙ₁—R², for which each R¹ independently comprises a group comprising an oligonucleotide, R² comprises an antibody and n1 is independently an integer of zero or greater that reflects the number of instances of R¹ in each compound (e.g., complex). In some embodiments, the fraction of compounds comprising a structure of formula (I): [R¹]ₙ₁—R², in a composition, for which n1 is zero, compared with all compounds of that structure in the composition for which n1 is one or greater, is less than 10%, less than 5%, less than 1% less than 0.5%, less than 0.1%, less than 0.05%, or less than 0.01%. As such, in some embodiments, the average value of n1 of complexes in a composition disclosed herein is in the range of 0.5 to 5 (e.g., 0.5-5, 1-5, 1-4, 1-3, 3-5, 0.5-4, 0.5-3, 0.5-2, 0.5-1.5, 0.5-1, 0.7-1.5, 1-1.6, 1-1.5, 1-1.4, 1-1.3, 1-1.2, 1.1-1.5, 0.8-2, 0.8-1.5, 0.8-1.3, 0.8-1.2, 0.8-1.1, 0.9-3, 0.9-2, 0.9-1.8, 0.9-1.6, 0.9-1.5, 0.9-1.4, 0.9-1.3, or 0.9-1.2). In some embodiments, the average value of n1 of complexes of the composition is 1.

In some embodiments, each instance of R¹ in a complex is covalently linked to a different amino acid residue of the antibody. In some embodiments, an amino acid to which R¹ is covalently linked comprises an ε-amino group (e.g., lysine, arginine). In some embodiments, an amino acid to which R¹ is covalently linked is a lysine. In some embodiments, an amino acid to which R¹ is covalently linked is a cysteine. In some embodiments, R¹ is directly covalently linked to an amino acid residue of the antibody. However, in some embodiments, R¹ is indirectly covalently linked to an amino acid of the antibody, e.g., covalently linked to a glycosylation site on the amino acid. In some embodiments, R¹ is not covalently linked to an amino acid residue residing in a CDR region of the antibody.

In some embodiments, complexes provided herein (e.g., in compositions or formulations described herein) comprise a structure of formula (I): [R¹]ₙ₁—R², in which each instance of R¹ independently comprises a group of the formula (Ia):

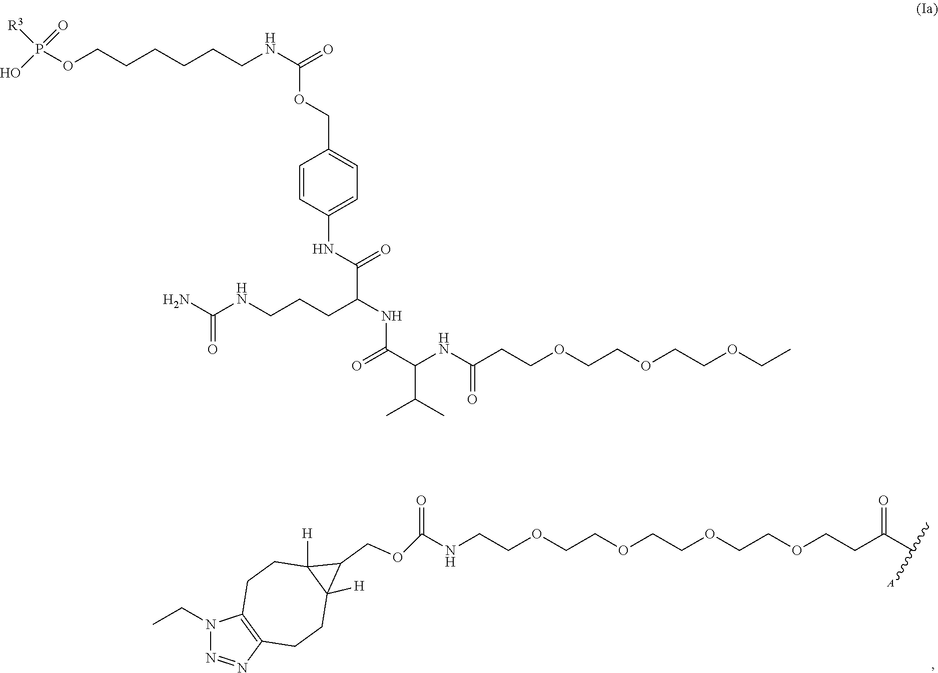

or a pharmaceutically acceptable salt thereof, in which R³ comprises an oligonucleotide, e.g., an RNAi oligonucleotide; and R¹ is covalently linked (e.g., indirectly or directly linked, e.g., directly linked) to R² at attachment point A. In some embodiments, R² comprises an antibody comprising a sequence as set forth in Table 2. For example, in some embodiments, R² comprises an antibody comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5, or 11, and a light chain complementarity determining region 3 (CDR-L3) comprising a sequence as set forth in SEQ ID NO: 6 or 16. In some embodiments, R² comprises an antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody comprising a VH comprising the amino acid sequence of SEQ ID NO: 17 and/or comprising a VL comprising the amino acid sequence of SEQ ID NO: 18. In some embodiments, R² comprises an antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)₂ fragment, an scFv, or an Fv. In some embodiments, R² comprises an antibody that is a Fab fragment. In some embodiments, R³ comprises an RNAi oligonucleotide (e.g., an siRNA) comprising an antisense strand comprising a nucleobase sequence of SEQ ID NO: 22 and a structure (5′→3′) of VP-mU*fG*mCmCmAmGmAmAmUmUmUmCmAfCmGmGmAmAmGmAmA*mC*mA, and a sense strand comprising a nucleobase sequence of SEQ ID NO: 21 and a structure (5′→3′) of mU*mU*mCmUfUmCmCmGfUfGfAmAmAfUmUmCmUmGfG*mC*mA, wherein: mA, mC, mG, and mU are 2′-O-methyl adenosine, 2′-O-methyl cytidine, 2′-O-methyl guanosine, and 2′-O-methyl uridine, respectively; fA, fC, fG, and fU are 2′-fluoro adenosine, 2′-fluoro cytidine, 2′-fluoro guanosine, and 2′-fluoro uridine, respectively; * between two nucleosides represents a phosphorothioate linkage; the absence of “*” between two nucleosides represents a phosphodiester internucleoside linkage; and “VP” represents 5′-(E)-Vinylphosphonate. In some embodiments, in each complex n1 is independently an integer (e.g., one or greater). In some embodiments, in each complex n1 is 1.

In some embodiments, complexes provided herein (e.g., in compositions or formulations described herein) comprise a structure of formula (I): [R¹]ₙ₁—R², in which each R¹ comprises a group of the formula (Ib):

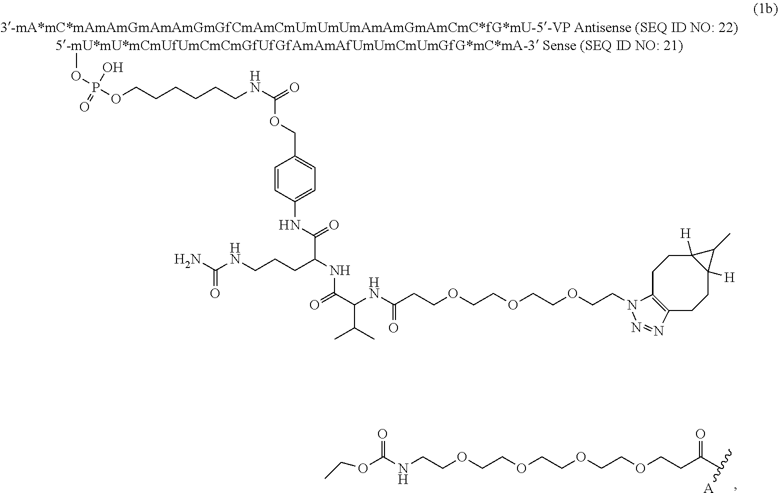

or a pharmaceutically acceptable salt thereof,
wherein: mA, mC, mG, and mU are 2′-O-methyl adenosine, 2′-O-methyl cytidine, 2′-O-methyl guanosine, and 2′-O-methyl uridine, respectively;
fA, fC, fG, and fU are 2′-fluoro adenosine, 2′-fluoro cytidine, 2′-fluoro guanosine, and 2′-fluoro uridine, respectively; * between two nucleosides represents a phosphorothioate linkage; the absence of “*” between two nucleosides represents a phosphodiester internucleoside linkage; and “VP” represents 5′-(E)-Vinylphosphonate; and wherein the oligonucleotide is an RNAi oligonucleotide (e.g., siRNA) comprising an antisense strand comprising a nucleobase sequence of UGCCAGAAUUUCACGGAAGAACA (SEQ ID NO: 22) and a sense strand comprising a nucleobase sequence of UUCUUCCGUGAAAUUCUGGCA (SEQ ID NO: 21); wherein n1 is an integer (e.g., one or greater) representing the number of instances of R¹ in each complex, and each R¹ is covalently linked to R² at attachment point A. In some embodiments, R² comprises an antibody comprising a sequence as set forth in Table 2. For example, in some embodiments, R² comprises an antibody comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5, or 11, and a light chain complementarity determining region 3 (CDR-L3) comprising a sequence as set forth in SEQ ID NO: 6 or 16. In some embodiments, R² comprises an antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody comprising a VH comprising the amino acid sequence of SEQ ID NO: 17 and/or comprising a VL comprising the amino acid sequence of SEQ ID NO: 18. In some embodiments, R² comprises an antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)₂ fragment, an scFv, or an Fv. In some embodiments, R² comprises an antibody that is a Fab fragment.

In some embodiments, complexes provided herein (e.g., in compositions or formulations described herein) comprise a structure of formula (I): [R¹]ₙ₁—R², in which each R¹ comprises a group of the formula (Ic), in which the antisense strand and the sense strand form a double stranded oligonucleotide:

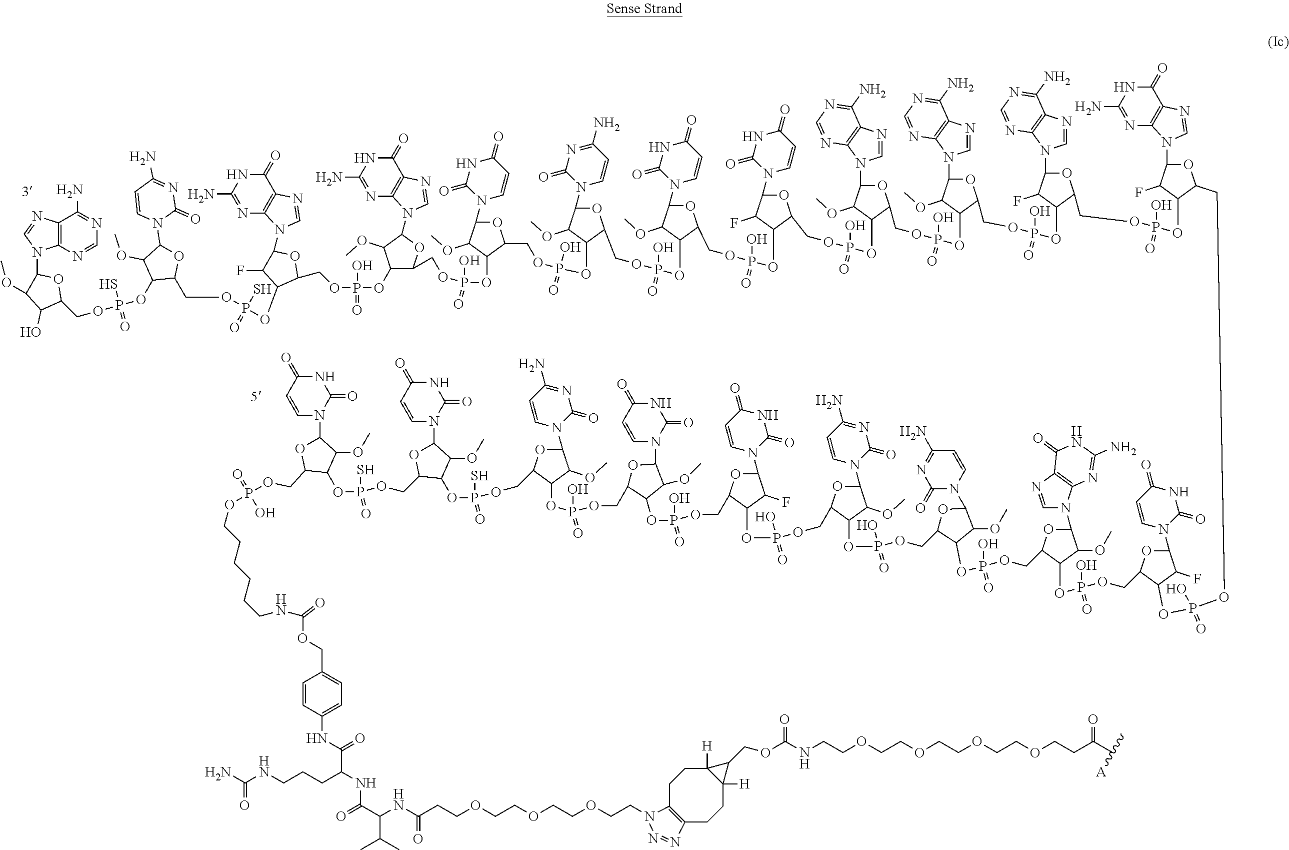

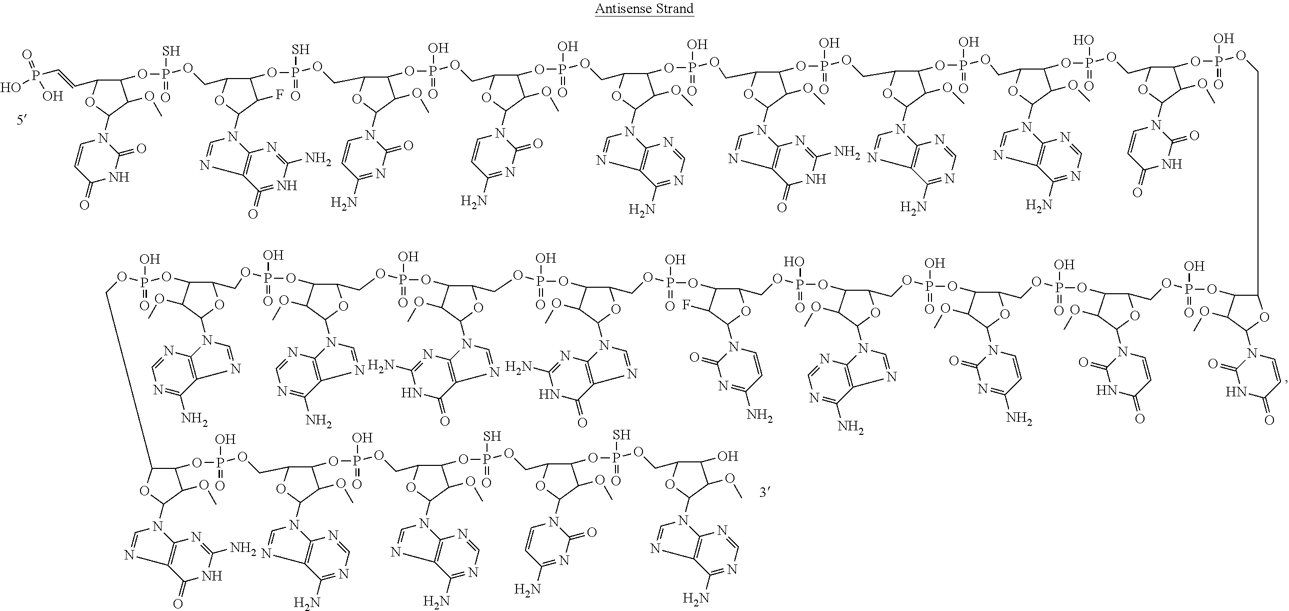

or a pharmaceutically acceptable salt thereof,
wherein R¹ is covalently linked to R² at attachment point A. In some embodiments, n1 is an integer (e.g., one or greater) representing the number of instances of R¹ in each complex. In some embodiments, R² comprises an antibody comprising a sequence as set forth in Table 2. For example, in some embodiments, R² comprises an antibody comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5, or 11, and a light chain complementarity determining region 3 (CDR-L3) comprising a sequence as set forth in SEQ ID NO: 6 or 16. In some embodiments, R² comprises an antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody comprising a VH comprising the amino acid sequence of SEQ ID NO: 17 and/or comprising a VL comprising the amino acid sequence of SEQ ID NO: 18. In some embodiments, R² comprises an antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)₂ fragment, an scFv, or an Fv. In some embodiments, R² comprises an antibody that is a Fab fragment.

In some embodiments, complexes provided herein (e.g., in compositions or formulations described herein) comprise a structure of the formula (Id):

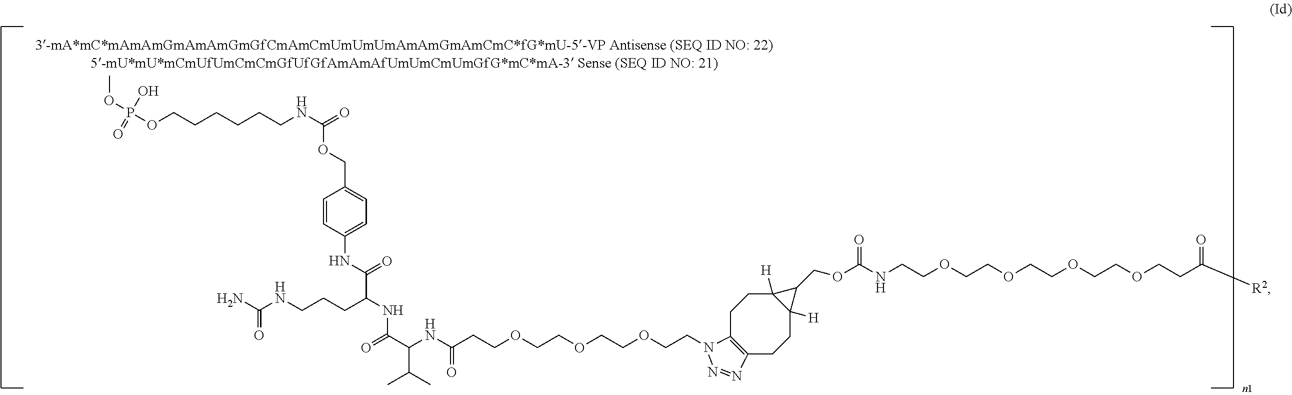

or a pharmaceutically acceptable salt thereof,
wherein: mA, mC, mG, and mU are 2′-O-methyl adenosine, 2′-O-methyl cytidine, 2′-O-methyl guanosine, and 2′-O-methyl uridine, respectively;
fA, fC, fG, and fU are 2′-fluoro adenosine, 2′-fluoro cytidine, 2′-fluoro guanosine, and 2′-fluoro uridine, respectively; * between two nucleosides represents a phosphorothioate linkage; the absence of “*” between two nucleosides represents a phosphodiester internucleoside linkage; and “VP” represents 5′-(E)-Vinylphosphonate; and wherein the oligonucleotide is an RNAi oligonucleotide (e.g., siRNA) comprising an antisense strand comprising a nucleobase sequence of UGCCAGAAUUUCACGGAAGAACA (SEQ ID NO: 22) and a sense strand comprising a nucleobase sequence of UUCUUCCGUGAAAUUCUGGCA (SEQ ID NO: 21);
wherein R² comprises an antibody comprising a sequence as set forth in Table 2; wherein n1 is an integer (e.g., one or greater) representing the number of instances of the group enclosed by square brackets, wherein each instance of the group enclosed by square brackets is covalently linked to a different amino acid residue of the antibody, optionally wherein each different amino acid residue is a lysine. In some embodiments, R² comprises an antibody comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5, or 11, and a light chain complementarity determining region 3 (CDR-L3) comprising a sequence as set forth in SEQ ID NO: 6 or 16. In some embodiments, R² 12030680.1 comprises an antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody comprising a VH comprising the amino acid sequence of SEQ ID NO: 17 and/or comprising a VL comprising the amino acid sequence of SEQ ID NO: 18. In some embodiments, R² comprises an antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)₂ fragment, an scFv, or an Fv. In some embodiments, R² comprises an antibody that is a Fab fragment.

Each of formula (Ia), formula (Ib), formula (Ic) and formula (Id), as depicted herein, include one or more phosphorous containing groups (e.g., phosphate group, phosphorothioate group, phosphodiester group) that are protonated. In certain embodiments, the phosphorous containing groups (e.g., phosphate group, phosphorothioate group, phosphodiester group) are as depicted, i.e., protonated. In certain embodiments, the phosphorous containing groups (e.g., phosphate group, phosphorothioate group, phosphodiester group) groups are deprotonated, i.e., in salt form. The extent to which the phosphorous containing groups are protonated or deprotonated will vary depending on the chemical environment of the complexes (e.g., pH, presence of basic media, etc.). For example, in a composition (e.g., formulation) described herein that has a pH of about 7.5, or at a physiological pH, complexes comprising a structure of formula (Ia), formula (Ib), formula (Ic) and formula (Id) may comprise one or more deprotonated phosphorous containing groups (e.g., a phosphate group, a phosphorothioate group, a phosphodiester group).

In some embodiments, complexes described herein comprise a structure of formula (A):

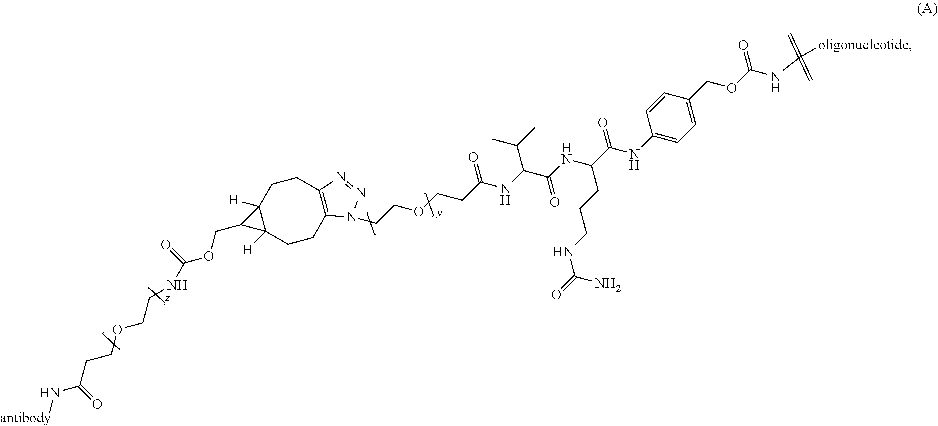

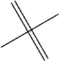

wherein y is 0-15 (e.g., 3), z is 0-15 (e.g., 4), and wherein represents an indirect or direct linkage. In some embodiments, the amide shown adjacent the antibody (e.g., anti-TfR1 antibody) in the structure (A) results from a reaction with an amine of the antibody, such as a lysine epsilon amine. In some embodiments, a complex described herein comprises an anti-TfR1 antibody (e.g., an anti-TfR1 Fab) covalently linked via a lysine of the antibody to the 5′ end of an oligonucleotide (e.g., an RNAi oligonucleotide). In some embodiments, the antibody comprises a sequence as set forth in Table 2. For example, in some embodiments, the antibody comprises a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprises a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5, or 11, and a light chain complementarity determining region 3 (CDR-L3) comprising a sequence as set forth in SEQ ID NO: 6 or 16. In some embodiments, the antibody comprises a heavy chain variable region (VH) comprising an amino acid sequence at least 85% (e.g., at least 95%) identical to SEQ ID NO: 17 and/or comprises a light chain variable region (VL) comprising an amino acid sequence at least 85% (e.g., at least 95%) identical to SEQ ID NO: 18. In some embodiments, the antibody comprises a VH comprising the amino acid sequence of SEQ ID NO: 17 and/or comprises a VL comprising the amino acid sequence of SEQ ID NO: 18. In some embodiments, the antibody comprises a heavy chain comprising an amino acid sequence at least 85% (e.g., at least 95%) identical to SEQ ID NO: 19 and/or comprises a light chain comprising an amino acid sequence at least 85% (e.g., at least 95%) identical to SEQ ID NO: 20. In some embodiments, the antibody comprises a heavy chain comprising the amino acid sequence of SEQ ID NO: 19 and/or comprises a light chain comprising the amino acid sequence of SEQ ID NO: 20. In some embodiments, the antibody is a Fab fragment, a full-length IgG, a Fab′ fragment, a F(ab′)₂ fragment, an scFv, or an Fv. In some embodiments, the antibody is a Fab fragment.

### Antibodies

In some embodiments, complexes described herein comprise an antibody that binds human transferrin receptor 1 (TfR1). An example human TfR1 amino acid sequence, corresponding to NCBI sequence NP_003225.2 (transferrin receptor protein 1 isoform 1, Homo sapiens) is as follows:

(SEQ ID NO: 23)
MMDQARSAFSNLFGGEPLSYTRFSLARQVDGDNSHVEMKLAVDEEENADN
NTKANVTKPKRCSGSICYGTIAVIVFFLIGFMIGYLGYCKGVEPKTECER
LAGTESPVREEPGEDFPAARRLYWDDLKRKLSEKLDSTDFTGTIKLLNEN
SYVPREAGSQKDENLALYVENQFREFKLSKVWRDQHFVKIQVKDSAQNSV
IIVDKNGRLVYLVENPGGYVAYSKAATVTGKLVHANFGTKKDFEDLYTPV
NGSIVIVRAGKITFAEKVANAESLNAIGVLIYMDQTKFPIVNAELSFFGH
AHLGTGDPYTPGFPSFNHTQFPPSRSSGLPNIPVQTISRAAAEKLFGNME
GDCPSDWKTDSTCRMVTSESKNVKLTVSNVLKEIKILNIFGVIKGFVEPD
HYVVVGAQRDAWGPGAAKSGVGTALLLKLAQMFSDMVLKDGFQPSRSIIF
ASWSAGDFGSVGATEWLEGYLSSLHLKAFTYINLDKAVLGTSNFKVSASP
LLYTLIEKTMQNVKHPVTGQFLYQDSNWASKVEKLTLDNAAFPFLAYSGI
PAVSFCFCEDTDYPYLGTTMDTYKELIERIPELNKVARAAAEVAGQFVIK
LTHDVELNLDYERYNSQLLSFVRDLNQYRADIKEMGLSLQWLYSARGDFF
RATSRLTTDFGNAEKTDRFVMKKLNDRVMRVEYHFLSPYVSPKESPFRHV
FWGSGSHTLPALLENLKLRKQNNGAFNETLFRNQLALATWTIQGAANALS
GDVWDIDNEF.

Table 2 provides examples of sequences of an anti-TfR1 antibody useful in the complexes provided herein.

TABLE 2
Examples of anti-TfR1 antibody sequences

| Feature | IMGT | Kabat | Chothia |
|---|---|---|---|
| CDR-H1 | GYSITSGYY | SGYYWN (SEQ ID | GYSITSGY |
| | (SEQ ID NO: 1) | NO: 7) | (SEQ ID NO: 12) |
| CDR-H2 | ITFDGAN | YITFDGANNYNPSLKN | FDG |
| | (SEQ ID NO: 2) | (SEQ ID NO: 8) | (SEQ ID NO: 13) |
| CDR-H3 | TRSSYDYDVL | SSYDYDVLDY | SYDYDVLD |
| | DY (SEQ ID | (SEQ ID NO: 9) | (SEQ ID NO: 14) |
| | NO: 3) | | |
| CDR-L1 | QDISNF (SEQ | RASQDISNFLN | SQDISNF |
| | ID NO: 4) | (SEQ ID NO: 10) | (SEQ ID NO: 15) |
| CDR-L2 | YTS (SEQ ID | YTSRLHS (SEQ ID | YTS |
| | NO: 5) | NO: 11) | (SEQ ID NO: 5) |
| CDR-L3 | QQGHTLPYT | QQGHTLPYT (SEQ | GHTLPY |
| | (SEQ ID NO: 6) | ID NO: 6) | (SEQ ID NO: 16) |

| | |
|---|---|
| VH | QVQLQESGPGLVKPSQTLSLTCTVTGYSITSGYYWNWIRQPPGKGLEWI |
| | GYITFDGANNYNPSLKNRVSISRDTSKNQFSLKLSSVTAEDTATYYCTR |
| | SSYDYDVLDYWGQGTTVTVSS (SEQ ID NO: 17) |
| VL | DIQMTQSPSSLSASVGDRVTITCRASQDISNFLNWYQQKPGQPVKLLIY |
| | YTSRLHSGVPSRFSGSGSGTDFTLTISSLQPEDFATYYCQQGHTLPYTFG |
| | QGTKLEIK (SEQ ID NO: 18) |
| Fab HC | QVQLQESGPGLVKPSQTLSLTCTVTGYSITSGYYWNWIRQPPGKGLEWI |
| | GYITFDGANNYNPSLKNRVSISRDTSKNQFSLKLSSVTAEDTATYYCTR |
| | SSYDYDVLDYWGQGTTVTVSSASTKGPSVFPLAPSSKSTSGGTAALGC |
| | LVKDYFPEPVTVSWNSGALTSGVHTFPAVLQSSGLYSLSSVVTVPSSSL |
| | GTQTYICNVNHKPSNTKVDKKVEPKSCDKTHT (SEQ ID NO: 19) |
| Fab LC | DIQMTQSPSSLSASVGDRVTITCRASQDISNFLNWYQQKPGQPVKLLIY |
| | YTSRLHSGVPSRFSGSGSGTDFTLTISSLQPEDFATYYCQQGHTLPYTFG |
| | QGTKLEIKRTVAAPSVFIFPPSDEQLKSGTASVVCLLNNFYPREAKVQW |
| | KVDNALQSGNSQESVTEQDSKDSTYSLSSTLTLSKADYEKHKVYACEV |
| | THQGLSSPVTKSFNRGEC (SEQ ID NO: 20) |

In some embodiments, the anti-TfR1 antibody of the present disclosure comprises a heavy chain complementarity determining region 1 (CDR-H1) of SEQ ID NO: 1 (according to the IMGT definition system), a heavy chain complementarity determining region 2 (CDR-H2) of SEQ ID NO: 2 (according to the IMGT definition system), a heavy chain complementarity determining region 3 (CDR-H3) of SEQ ID NO: 3 (according to the IMGT definition system), a light chain complementarity determining region 1 (CDR-L1) of SEQ ID NO: 4 (according to the IMGT definition system), a light chain complementarity determining region 2 (CDR-L2) of SEQ ID NO: 5 (according to the IMGT definition system), and a light chain complementarity determining region 3 (CDR-L3) of SEQ ID NO: 6 (according to the IMGT definition system).

In some embodiments, the anti-TfR1 antibody of the present disclosure comprises a heavy chain complementarity determining region 1 (CDR-H1) of SEQ ID NO: 7 (according to the Kabat definition system), a heavy chain complementarity determining region 2 (CDR-H2) of SEQ ID NO: 8 (according to the Kabat definition system), a heavy chain complementarity determining region 3 (CDR-H3) of SEQ ID NO: 9 (according to the Kabat definition system), a light chain complementarity determining region 1 (CDR-L1) of SEQ ID NO: 10 (according to the Kabat definition system), a light chain complementarity determining region 2 (CDR-L2) of SEQ ID NO: 11 (according to the Kabat definition system), and a light chain complementarity determining region 3 (CDR-L3) of SEQ ID NO: 6 (according to the Kabat definition system).

In some embodiments, the anti-TfR1 antibody of the present disclosure comprises a heavy chain complementarity determining region 1 (CDR-H1) of SEQ ID NO: 12 (according to the Chothia definition system), a heavy chain complementarity determining region 2 (CDR-H2) of SEQ ID NO: 13 (according to the Chothia definition system), a heavy chain complementarity determining region 3 (CDR-H3) of SEQ ID NO: 14 (according to the Chothia definition system), a light chain complementarity determining region 1 (CDR-L1) of SEQ ID NO: 15 (according to the Chothia definition system), a light chain complementarity determining region 2 (CDR-L2) of SEQ ID NO: 5 (according to the Chothia definition system), and a light chain complementarity determining region 3 (CDR-L3) of SEQ ID NO: 16 (according to the Chothia definition system).

In some embodiments, the anti-TfR1 antibody of the present disclosure comprises a heavy chain variable region (VH) containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VH comprising the amino acid sequence of SEQ ID NO: 17. Alternatively or in addition (e.g., in addition), the anti-TfR1 antibody of the present disclosure comprises a light chain variable region (VL) containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VL comprising the amino acid sequence of SEQ ID NO: 18.

In some embodiments, the anti-TfR1 antibody of the present disclosure comprises a VH comprising an amino acid sequence that is at least 75% (e.g., 75%, 80%, 85%, 90%, 95%, 98%, or 99%) identical in the framework regions to the VH comprising the amino acid sequence of SEQ ID NO: 17. Alternatively or in addition (e.g., in addition), in some embodiments, the anti-TfR1 antibody of the present disclosure comprises a VL comprising an amino acid sequence that is at least 75% (e.g., 75%, 80%, 85%, 90%, 95%, 98%, or 99%) identical in the framework regions to the VL comprising the amino acid sequence of SEQ ID NO: 18.

In some embodiments, the anti-TfR1 antibody of the present disclosure comprises a VH comprising the amino acid sequence of SEQ ID NO: 17. Alternatively or in addition (e.g., in addition), in some embodiments, the anti-TfR1 antibody of the present disclosure comprises a VL comprising the amino acid sequence of SEQ ID NO: 18.

In some embodiments, the anti-TfR1 antibody of the present disclosure comprises a heavy chain comprising an amino acid sequence least 75% (e.g., 75%, 80%, 85%, 90%, 95%, 98%, or 99%) identical to the amino acid sequence of SEQ ID NO: 19. Alternatively or in addition (e.g., in addition), the anti-TfR1 antibody of the present disclosure comprises a light chain comprising an amino acid sequence least 75% (e.g., 75%, 80%, 85%, 90%, 95%, 98%, or 99%) identical to the amino acid sequence of SEQ ID NO: 20. In some embodiments, the anti-TfR1 antibody of the present disclosure is a Fab that comprises a heavy chain comprising an amino acid sequence least 75% (e.g., 75%, 80%, 85%, 90%, 95%, 98%, or 99%) identical to the amino acid sequence of SEQ ID NO: 19. Alternatively or in addition (e.g., in addition), the anti-TfR1 antibody of the present disclosure is a Fab that comprises a light chain comprising an amino acid sequence least 75% (e.g., 75%, 80%, 85%, 90%, 95%, 98%, or 99%) identical to the amino acid sequence of SEQ ID NO: 20.

In some embodiments, the anti-TfR1 antibody of the present disclosure comprises a heavy chain comprising the amino acid sequence of SEQ ID NO: 19. Alternatively or in addition (e.g., in addition), the anti-TfR1 antibody of the present disclosure comprises a light chain comprising the amino acid sequence of SEQ ID NO: 20. In some embodiments, the anti-TfR1 antibody of the present disclosure is a Fab that comprises a heavy chain comprising an amino acid sequence of SEQ ID NO: 19. Alternatively or in addition (e.g., in addition), the anti-TfR1 antibody of the present disclosure is a Fab that comprises a light chain comprising the amino acid sequence of SEQ ID NO: 20.

In some embodiments, the anti-TfR1 antibody provided herein may have one or more post-translational modifications. In some embodiments, N-terminal cyclization, also called pyroglutamate formation (pyro-Glu), may occur in the antibody at N-terminal Glutamate (Glu) and/or Glutamine (Gln) residues during production. As such, it should be appreciated that an antibody specified as having a sequence comprising an N-terminal glutamate or glutamine residue encompasses antibodies that have undergone pyroglutamate formation resulting from a post-translational modification. In some embodiments, pyroglutamate formation occurs in a heavy chain sequence. In some embodiments, pyroglutamate formation occurs in a light chain sequence.

### Oligonucleotides

In some embodiments, the oligonucleotides provided herein are useful for targeting DUX4 (e.g., for reducing expression or activity of DUX4, such as the level of DUX4 protein and/or mRNA). In some embodiments, the oligonucleotides are designed to cause RNAi mediated degradation of DUX4 mRNA. In some embodiments, the oligonucleotide is an RNAi oligonucleotide. In some embodiments, the oligonucleotide provided herein comprises an antisense strand that comprises a region of complementarity to a DUX4 RNA. In some embodiments, the oligonucleotide provided herein further comprises a sense strand that forms a double-stranded oligonucleotide (e.g., siRNA). In some embodiments, the oligonucleotide is designed to have desirable bioavailability and/or serum-stability properties. In some embodiments, the oligonucleotide is designed to have desirable binding affinity properties. In some embodiments, the oligonucleotide is designed to have desirable toxicity profiles. In some embodiments, the oligonucleotide is designed to have low-complement activation and/or cytokine induction properties. In some embodiments, the oligonucleotide is designed to have reduced off-target effects, e.g., compared to other known DUX4-targeting siRNAs.

Examples of oligonucleotides useful for targeting DUX4 are provided in U.S. Pat. No. 9,988,628, published on Feb. 2, 2017, entitled “AGENTS USEFUL IN TREATING FACIOSCAPULOHUMERAL MUSCULAR DYSTROPHY”; U.S. Pat. No. 9,469,851, published Oct. 30, 2014, entitled “RECOMBINANT VIRUS PRODUCTS AND METHODS FOR INHIBITING EXPRESSION OF DUX4”; US Patent Application Publication 20120225034, published on Sep. 6, 2012, entitled “AGENTS USEFUL IN TREATING FACIOSCAPULOHUMERAL MUSCULAR DYSTROPHY”; PCT Patent Application Publication Number WO 2013/120038, published on Aug. 15, 2013, entitled “MORPHOLINO TARGETING DUX4 FOR TREATING FSHD”; Chen et al., “Morpholino-mediated Knockdown of DUX4 Toward Facioscapulohumeral Muscular Dystrophy Therapeutics,” Molecular Therapy, 2016, 24:8, 1405-1411; and Ansseau et al., “Antisense Oligonucleotides Used to Target the DUX4 mRNA as Therapeutic Approaches in Facioscapulohumeral Muscular Dystrophy (FSHD),” Genes, 2017, 8, 93; the contents of each of which are incorporated herein in their entireties. In some embodiments, oligonucleotides may comprise a region of complementarity to a sequence as set forth as: Human DUX4, corresponding to NCBI sequence NM_001293798.2 (SEQ ID NO: 25) or NCBI Sequence: NM_001306068.3 (SEQ ID NO: 26) as below and/or (e.g., and) Mouse DUX4, corresponding to NCBI sequence NM_001081954.1 (SEQ ID NO: 27), as below. Other non-limiting exemplary human DUX4 RNA include NCBI Sequence: NM_033178, GenBank accession numbers FJ439133, AF117653, HM101229, HM101230, HM101232, HM101233, HM101234, HM101235, HM101240, HM101241, HM101242, HM101243, HM101244, HM101245, HM101246, HM101247, HM101248, HM101249, HM101250, HM101251 and HM190160, HM190161, HM190162, HM190163, HM190164, HM190165, HM190166, HM190167, HM190168, HM190169, HM190170, HM190171, HM190172, HM190173, HM190174, HM190175, HM190176, HM190177, HM190178, HM190179, HM190180, HM190181, HM190182, HM190183, HM190184, HM190185, HM190186, HM190187, HM190188, HM190189, HM190190, HM190191, HM190192, HM190193, HM190194, HM190195, HM190196, each of which is incorporated herein by reference. In some embodiments, the oligonucleotide may have a region of complementarity to a hypomethylated, contracted D4Z4 repeat, as in Daxinger, et al., “Genetic and Epigenetic Contributors to FSHD,” published in Curr Opin Genet Dev in 2015, Lim J-W, et al., DICER/AGO-dependent epigenetic silencing of D4Z4 repeats enhanced by exogenous siRNA suggests mechanisms and therapies for FSHD Hum Mol Genet. 2015 Sep. 1; 24(17): 4817-4828, the contents of each of which are incorporated in their entireties.

In some embodiments, oligonucleotides may comprise a region of complementarity to a sequence set forth as follows, which is an example human DUX4 gene sequence (NM_001293798.2) (SEQ ID NO: 25):

ATGGCCCTCCCGACACCCTCGGACAGCACCCTCCCCGCGGAAGCCCGGGGACGAG
GACGGCGACGGAGACTCGTTTGGACCCCGAGCCAAAGCGAGGCCCTGCGAGCCTG
CTTTGAGCGGAACCCGTACCCGGGCATCGCCACCAGAGAACGGCTGGCCCAGGCC
ATCGGCATTCCGGAGCCCAGGGTCCAGATTTGGTTTCAGAATGAGAGGTCACGCC
AGCTGAGGCAGCACCGGCGGGAATCTCGGCCCTGGCCCGGGAGACGCGGCCCGCC
AGAAGGCCGGCGAAAGCGGACCGCCGTCACCGGATCCCAGACCGCCCTGCTCCTC
CGAGCCTTTGAGAAGGATCGCTTTCCAGGCATCGCCGCCCGGGAGGAGCTGGCCA
GAGAGACGGGCCTCCCGGAGTCCAGGATTCAGATCTGGTTTCAGAATCGAAGGGC
CAGGCACCCGGGACAGGGTGGCAGGGCGCCCGCGCAGGCAGGCGGCCTGTGCAG
CGCGGCCCCCGGCGGGGGTCACCCTGCTCCCTCGTGGGTCGCCTTCGCCCACACCG
GCGCGTGGGGAACGGGGCTTCCCGCACCCCACGTGCCCTGCGCGCCTGGGGCTCT
CCCACAGGGGGCTTTCGTGAGCCAGGCAGCGAGGGCCGCCCCCGCGCTGCAGCCC
AGCCAGGCCGCGCCGGCAGAGGGGATCTCCCAACCTGCCCCGGCGCGCGGGGATT
TCGCCTACGCCGCCCCGGCTCCTCCGGACGGGGCGCTCTCCCACCCTCAGGCTCCT
CGCTGGCCTCCGCACCCGGGCAAAAGCCGGGAGGACCGGGACCCGCAGCGCGAC
GGCCTGCCGGGCCCCTGCGCGGTGGCACAGCCTGGGCCCGCTCAAGCGGGGCCGC
AGGGCCAAGGGGTGCTTGCGCCACCCACGTCCCAGGGGAGTCCGTGGTGGGGCTG
GGGCCGGGGTCCCCAGGTCGCCGGGGCGGCGTGGGAACCCCAAGCCGGGGCAGC
TCCACCTCCCCAGCCCGCGCCCCCGGACGCCTCCGCCTCCGCGCGGCAGGGGCAG
ATGCAAGGCATCCCGGCGCCCTCCCAGGCGCTCCAGGAGCCGGCGCCCTGGTCTG
CACTCCCCTGCGGCCTGCTGCTGGATGAGCTCCTGGCGAGCCCGGAGTTTCTGCAG
CAGGCGCAACCTCTCCTAGAAACGGAGGCCCCGGGGGAGCTGGAGGCCTCGGAA
GAGGCCGCCTCGCTGGAAGCACCCCTCAGCGAGGAAGAATACCGGGCTCTGCTGG
AGGAGCTTTAGGACGCGGGGTCTAGGCCCGGTGAGAGACTCCACACCGCGGAGAA
CTGCCATTCTTTCCTGGGCATCCCGGGGATCCCAGAGCCGGCCCAGGTACCAGCAG
ACCTGCGCGCAGTGCGCACCCCGGCTGACGTGCAAGGGAGCTCGCTGGCCTCTCT
GTGCCCTTGTTCTTCCGTGAAATTCTGGCTGAATGTCTCCCCCCACCTTCCGACGCT
GTCTAGGCAAACCTGGATTAGAGTTACATCTCCTGGATGATTAGTTCAGAGATATA
TTAAAATGCCCCCTCCCTGTGGATCCTATAG

In some embodiments, oligonucleotides may comprise a region of complementarity to a sequence set forth as follows, which is an example human DUX4 gene sequence (NM_001306068.3) (SEQ ID NO: 26):

ATGGCCCTCCCGACACCCTCGGACAGCACCCTCCCCGCGGAAGCCCGGGGACGAG
GACGGCGACGGAGACTCGTTTGGACCCCGAGCCAAAGCGAGGCCCTGCGAGCCTG
CTTTGAGCGGAACCCGTACCCGGGCATCGCCACCAGAGAACGGCTGGCCCAGGCC
ATCGGCATTCCGGAGCCCAGGGTCCAGATTTGGTTTCAGAATGAGAGGTCACGCC
AGCTGAGGCAGCACCGGCGGGAATCTCGGCCCTGGCCCGGGAGACGCGGCCCGCC
AGAAGGCCGGCGAAAGCGGACCGCCGTCACCGGATCCCAGACCGCCCTGCTCCTC
CGAGCCTTTGAGAAGGATCGCTTTCCAGGCATCGCCGCCCGGGAGGAGCTGGCCA
GAGAGACGGGCCTCCCGGAGTCCAGGATTCAGATCTGGTTTCAGAATCGAAGGGC
CAGGCACCCGGGACAGGGTGGCAGGGCGCCCGCGCAGGCAGGCGGCCTGTGCAG
CGCGGCCCCCGGCGGGGGTCACCCTGCTCCCTCGTGGGTCGCCTTCGCCCACACCG
GCGCGTGGGGAACGGGGCTTCCCGCACCCCACGTGCCCTGCGCGCCTGGGGCTCT
CCCACAGGGGGCTTTCGTGAGCCAGGCAGCGAGGGCCGCCCCCGCGCTGCAGCCC
AGCCAGGCCGCGCCGGCAGAGGGGATCTCCCAACCTGCCCCGGCGCGCGGGGATT
TCGCCTACGCCGCCCCGGCTCCTCCGGACGGGGCGCTCTCCCACCCTCAGGCTCCT
CGGTGGCCTCCGCACCCGGGCAAAAGCCGGGAGGACCGGGACCCGCAGCGCGAC
GGCCTGCCGGGCCCCTGCGCGGTGGCACAGCCTGGGCCCGCTCAAGCGGGGCCGC
AGGGCCAAGGGGTGCTTGCGCCACCCACGTCCCAGGGGAGTCCGTGGTGGGGCTG
GGGCCGGGGTCCCCAGGTCGCCGGGGCGGCGTGGGAACCCCAAGCCGGGGCAGC
TCCACCTCCCCAGCCCGCGCCCCCGGACGCCTCCGCCTCCGCGCGGCAGGGGCAG
ATGCAAGGCATCCCGGCGCCCTCCCAGGCGCTCCAGGAGCCGGCGCCCTGGTCTG
CACTCCCCTGCGGCCTGCTGCTGGATGAGCTCCTGGCGAGCCCGGAGTTTCTGCAG
CAGGCGCAACCTCTCCTAGAAACGGAGGCCCCGGGGGAGCTGGAGGCCTCGGAA
GAGGCCGCCTCGCTGGAAGCACCCCTCAGCGAGGAAGAATACCGGGCTCTGCTGG
AGGAGCTTTAGGACGCGGGGTTGGGACGGGGTCGGGTGGTTCGGGGCAGGGCGGT
GGCCTCTCTTTCGCGGGGAACACCTGGCTGGCTACGGAGGGGCGTGTCTCCGCCCC
GCCCCCTCCACCGGGCTGACCGGCCTGGGATTCCTGCCTTCTAGGTCTAGGCCCGG
TGAGAGACTCCACTCCGCGGAGAACTGCCTTTCTTTCCTGGGCATCCCGGGGATCC
CAGAGCCGGCCCAGGTACCAGCAGACCTGCGCGCAGTGCGCACCCCGGCTGACGT
GCAAGGGAGCTCGCTGGCCTCTCTGTGCCCTTGTTCTTCCGTGAAATTCTGGCTGA
ATGTCTCCCCCCACCTTCCGACGCTGTCTAGGCAAACCTGGATTAGAGTTACATCT
CCTGGATGATTAGTTCAGAGATATATTAAAATGCCCCCTCCCTGTGGATCCTATAG

In some embodiments, oligonucleotides may comprise a region of complementarity to a sequence set forth as follows, which is an example mouse DUX4 gene sequence (SEQ ID NO: 27) (NM_001081954.1):

ATGGCAGAAGCTGGCAGCCCTGTTGGTGGCAGTGGTGTGGCACGGGAATCCCGGC
GGCGCAGGAAGACGGTTTGGCAGGCCTGGCAAGAGCAGGCCCTGCTATCAACTTT
CAAGAAGAAGAGATACCTGAGCTTCAAGGAGAGGAAGGAGCTGGCCAAGCGAAT
GGGGGTCTCAGATTGCCGCATCCGCGTGTGGTTTCAGAACCGCAGGAATCGCAGT
GGAGAGGAGGGGCATGCCTCAAAGAGGTCCATCAGAGGCTCCAGGCGGCTAGCCT
CGCCACAGCTCCAGGAAGAGCTTGGATCCAGGCCACAGGGTAGAGGCATGCGCTC
ATCTGGCAGAAGGCCTCGCACTCGACTCACCTCGCTACAGCTCAGGATCCTAGGG
CAAGCCTTTGAGAGGAACCCACGACCAGGCTTTGCTACCAGGGAGGAGCTGGCGC
GTGACACAGGGTTGCCCGAGGACACGATCCACATATGGTTTCAAAACCGAAGAGC
TCGGCGGCGCCACAGGAGGGGCAGGCCCACAGCTCAAGATCAAGACTTGCTGGCG
TCACAAGGGTCGGATGGGGCCCCTGCAGGTCCGGAAGGCAGAGAGCGTGAAGGT
GCCCAGGAGAACTTGTTGCCACAGGAAGAAGCAGGAAGTACGGGCATGGATACCT
CGAGCCCTAGCGACTTGCCCTCCTTCTGCGGAGAGTCCCAGCCTTTCCAAGTGGCA
CAGCCCCGTGGAGCAGGCCAACAAGAGGCCCCCACTCGAGCAGGCAACGCAGGC
TCTCTGGAACCCCTCCTTGATCAGCTGCTGGATGAAGTCCAAGTAGAAGAGCCTGC
TCCAGCCCCTCTGAATTTGGATGGAGACCCTGGTGGCAGGGTGCATGAAGGTTCCC
AGGAGAGCTTTTGGCCACAGGAAGAAGCAGGAAGTACAGGCATGGATACTTCTAG
CCCCAGCGACTCAAACTCCTTCTGCAGAGAGTCCCAGCCTTCCCAAGTGGCACAGC
CCTGTGGAGCGGGCCAAGAAGATGCCCGCACTCAAGCAGACAGCACAGGCCCTCT
GGAACTCCTCCTCCTTGATCAACTGCTGGACGAAGTCCAAAAGGAAGAGCATGTG
CCAGTCCCACTGGATTGGGGTAGAAATCCTGGCAGCAGGGAGCATGAAGGTTCCC
AGGACAGCTTACTGCCCCTGGAGGAAGCAGTAAATTCGGGCATGGATACCTCGAT
CCCTAGCATCTGGCCAACCTTCTGCAGAGAATCCCAGCCTCCCCAAGTGGCACAGC
CCTCTGGACCAGGCCAAGCACAGGCCCCCACTCAAGGTGGGAACACGGACCCCCT
GGAGCTCTTCCTCTATCAACTGTTGGATGAAGTCCAAGTAGAAGAGCATGCTCCAG
CCCCTCTGAATTGGGATGTAGATCCTGGTGGCAGGGTGCATGAAGGTTCGTGGGA
GAGCTTTTGGCCACAGGAAGAAGCAGGAAGTACAGGCCTGGATACTTCAAGCCCC
AGCGACTCAAACTCCTTCTTCAGAGAGTCCAAGCCTTCCCAAGTGGCACAGCGCC
GTGGAGCGGGCCAAGAAGATGCCCGCACTCAAGCAGACAGCACAGGCCCTCTGG
AACTCCTCCTCTTTGATCAACTGCTGGACGAAGTCCAAAAGGAAGAGCATGTGCC
AGCCCCACTGGATTGGGGTAGAAATCCTGGCAGCATGGAGCATGAAGGTTCCCAG
GACAGCTTACTGCCCCTGGAGGAAGCAGCAAATTCGGGCAGGGATACCTCGATCC
CTAGCATCTGGCCAGCCTTCTGCAGAAAATCCCAGCCTCCCCAAGTGGCACAGCCC
TCTGGACCAGGCCAAGCACAGGCCCCCATTCAAGGTGGGAACACGGACCCCCTGG
AGCTCTTCCTTGATCAACTGCTGACCGAAGTCCAACTTGAGGAGCAGGGGCCTGCC
CCTGTGAATGTGGAGGAAACATGGGAGCAAATGGACACAACACCTATCTGCCTCT
CACTTCAGAAGAATATCAGACTCTTCTAGATATGCTCTGA

In some embodiments, an oligonucleotide may comprise a region of complementarity to DUX4 sequences of multiple species, e.g., selected from human, mouse and non-human species (e.g., cynomolgus monkey).

In some embodiments, an oligonucleotide provided herein is a double-stranded RNAi oligonucleotide (e.g., siRNA) targeting DUX4. In some embodiments, a DUX4-targeting RNAi oligonucleotide is 7, 8, 9, 10, 11, 12, 13, 14, 15, 16, 17, 18, 19, 20, 21, 22, 23, 24, 25, 26, 27, 28, 29, 30, 35, 40, 45, 50, or more base pairs in length. In some embodiments, a DUX4-targeting RNAi oligonucleotide is 8 to 30 base pairs in length, 10 to 15 base pairs in length, 10 to 20 base pairs in length, 15 to 25 base pairs in length, 19 to 21 base pairs in length, 21 to 23 base pairs in length.

In some embodiments, an oligonucleotide described herein is a double stranded RNAi oligonucleotide (e.g., an siRNA) comprising an antisense strand and a sense strand. In some embodiments, the antisense strand is 18-25 (e.g., 18, 19, 20, 21, 22, 23, 24, or 25) nucleosides in length. In some embodiments, the antisense strand is 23 nucleosides in length. In some embodiments, the sense strand is 18-25 (e.g., 18, 19, 20, 21, 22, 23, 24, or 25) nucleosides in length. In some embodiments, the sense strand is 21 nucleosides in length.

In some embodiments, an oligonucleotide described herein is a double stranded RNAi oligonucleotide (e.g., an siRNA) comprising an antisense strand comprising a region of complementarity of at least 16 (e.g., 16, 17, 18, 19, 20, or more) consecutive nucleosides to a DUX4 sequence as set forth in SEQ ID NO: 25, 26, and/or 27. In some embodiments, the antisense strand comprises a region of complementarity of at least 16 (e.g., 16, 17, 18, 19, 20, or more) consecutive nucleosides to a target sequence as set forth in SEQ ID NO: 24 or 21. In some embodiments, the antisense strand comprises a region of complementarity of at least 16 (e.g., 16, 17, 18, 19, 20, or more) consecutive nucleosides to a target sequence as set forth in SEQ ID NO: 28.

In some embodiments, an oligonucleotide described herein is a double stranded RNAi oligonucleotide (e.g., an siRNA) comprising an antisense strand that is 18-25 nucleosides (e.g., 18, 19, 20, 21, 22, 23, 24, or 25 nucleosides) in length and comprising a region of complementarity to a target sequence as set forth in SEQ ID NO: 24 (UGUUCUUCCGUGAAAUUCUGGCA), wherein the region of complementarity is at least 16 nucleosides (e.g., 16, 17, 18, 19, 20, 21, or 22 nucleosides) in length. In some embodiments, the antisense strand is 23 nucleosides in length and comprises a region of complementarity to a target sequence as set forth in SEQ ID NO: 24 (UGUUCUUCCGUGAAAUUCUGGCA), wherein the region of complementarity is at least 20 nucleosides (e.g., 20, 21, or 22 nucleosides) in length. In some embodiments, an oligonucleotide described herein is a double stranded RNAi oligonucleotide (e.g., an siRNA) comprising an antisense strand that is 18-25 nucleosides (e.g., 18, 19, 20, 21, 22, 23, 24, or 25 nucleosides) in length and comprising a region of complementarity to a target sequence as set forth in SEQ ID NO: 28 (UGUUCUUCCGUGAAAUUCUGGCU), wherein the region of complementarity is at least 16 nucleosides (e.g., 16, 17, 18, 19, 20, 21, or 22 nucleosides) in length. In some embodiments, the antisense strand is 23 nucleosides in length and comprises a region of complementarity to a target sequence as set forth in SEQ ID NO: 28 (UGUUCUUCCGUGAAAUUCUGGCU), wherein the region of complementarity is at least 20 nucleosides (e.g., 20, 21, or 22 nucleosides) in length. In some embodiments, the region of complementarity is fully complementarity with all or a portion of its target sequence. In some embodiments, the region of complementarity includes 1, 2, 3 or more mismatches.

In some embodiments, an oligonucleotide described herein is a double stranded RNAi oligonucleotide (e.g., an siRNA) comprising an antisense strand that comprises at least 15 consecutive nucleosides of (e.g., at least 15, at least 16, at least 17, at least 18, at least 19, or at least 20) the nucleobase sequence of SEQ ID NO: 22 (UGCCAGAAUUUCACGGAAGAACA). In some embodiments, the oligonucleotide further comprises a sense strand comprising at least 15 (e.g., 15, 16, 17, 18, 19, 20, or more) consecutive nucleosides complementary to the antisense strand. In some embodiments, the oligonucleotide further comprises a sense strand that comprises at least 15 consecutive nucleosides of (e.g., at least 15, at least 16, at least 17, at least 18, at least 19, or at least 20) the nucleobase sequence of SEQ ID NO: 21 (UUCUUCCGUGAAAUUCUGGCA).

In some embodiments, an oligonucleotide described herein is a double stranded RNAi oligonucleotide (e.g., an siRNA) comprising an antisense strand that comprises the nucleobase sequence of SEQ ID NO: 22 (UGCCAGAAUUUCACGGAAGAACA). In some embodiments, the oligonucleotide further comprises a sense strand that comprises the nucleobase sequence of SEQ ID NO: 21 (UUCUUCCGUGAAAUUCUGGCA).

In some embodiments, an oligonucleotide described herein is a double stranded RNAi oligonucleotide (e.g., an siRNA) comprising an antisense strand that comprises the nucleobase sequence of SEQ ID NO: 22 (UGCCAGAAUUUCACGGAAGAACA) and a sense strand that hybridizes to the antisense strand and comprises the nucleobase sequence of SEQ ID NO: 21 (UUCUUCCGUGAAAUUCUGGCA), wherein the antisense strand and/or (e.g., and) the sense strand comprises one or more modified nucleosides (e.g., 2′-modified nucleosides). In some embodiment, the one or more modified nucleosides are selected from 2′-O-Me and 2′-F modified nucleosides. In some embodiments, the antisense strand further comprises a 5′-(E)-vinylphosphonate.

In some embodiments, an oligonucleotide described herein is a double stranded RNAi oligonucleotide (e.g., an siRNA) comprising an antisense strand that comprises the nucleobase sequence of SEQ ID NO: 22 (UGCCAGAAUUUCACGGAAGAACA) and a sense strand that hybridizes to the antisense strand and comprises the nucleobase sequence of SEQ ID NO: 21 (UUCUUCCGUGAAAUUCUGGCA), wherein each nucleoside in the antisense strand and/or (e.g., and) each nucleoside in the sense strand is a 2′-modified nucleoside selected from 2′-O-Me and 2′-F modified nucleosides. In some embodiments, nucleosides at three consecutive positions of the sense strand are 2′-F modified nucleosides and the nucleosides at three consecutive positions of the antisense strand are 2′-O-Me modified nucleosides. In some embodiments, the antisense strand further comprises a 5′-(E)-vinylphosphonate.

In some embodiments, an oligonucleotide described herein is a double stranded RNAi oligonucleotide (e.g., an siRNA) comprising an antisense strand that comprises the nucleobase sequence of SEQ ID NO: 22 (UGCCAGAAUUUCACGGAAGAACA) and a sense strand that hybridizes to the antisense strand and comprises the nucleobase sequence of SEQ ID NO: 21 (UUCUUCCGUGAAAUUCUGGCA), wherein each nucleoside in the antisense strand and each nucleoside in the sense strand is a 2′-modified nucleoside selected from 2′-O-Me and 2′-F modified nucleosides, wherein the nucleosides at three consecutive positions of the sense strand are 2′-F modified nucleosides and the nucleosides at three consecutive positions of the antisense strand are 2′-O-Me modified nucleosides, and wherein the antisense strand and/or (e.g., and) the sense strand each comprises one or more phosphorothioate internucleoside linkages. In some embodiments, the antisense strand further comprises a 5′-(E)-vinylphosphonate.

In some embodiments, the sense strand does not comprise any phosphorothioate internucleoside linkages (all the internucleoside linkages in the sense strand are phosphodiester internucleoside linkages), and the antisense strand comprises 1, 2, or 3 phosphorothioate internucleoside linkages. In some embodiments, the sense strand comprises 2 phosphorothioate internucleoside linkages, and the antisense strand comprises 4 phosphorothioate internucleoside linkages. In some embodiments, the sense strand comprises 4 phosphorothioate internucleoside linkages, and the antisense strand comprises 4 phosphorothioate internucleoside linkages.

In some embodiments, the antisense strand comprises 2 phosphorothioate internucleoside linkages, optionally wherein the two internucleoside linkages at the 3′ end of the antisense strand are phosphorothioate internucleoside linkages and the rest of the internucleoside linkages in the antisense strand are phosphodiester internucleoside linkages. In some embodiments, the antisense strand comprises 4 phosphorothioate internucleoside linkages, optionally wherein the two internucleoside linkages at the 3′ end and the two internucleoside linkage at the 5′ end of the antisense strand are phosphorothioate internucleoside linkages and the rest of the internucleoside linkages in the antisense strand are phosphodiester internucleoside linkages. In some embodiments, the sense strand comprises 2 phosphorothioate internucleoside linkages, optionally wherein the two internucleoside linkages at the 5′ end of the sense strand are phosphorothioate internucleoside linkages and the rest of the internucleoside linkages in the sense strand are phosphodiester internucleoside linkages. In some embodiments, the sense strand comprises 4 phosphorothioate internucleoside linkages. In some embodiments, the two internucleoside linkages at the 5′ end and the two internucleoside linkages at the 3′ end of the sense strand are phosphorothioate internucleoside linkages, and the rest of the internucleoside linkages in the sense strand are phosphodiester internucleoside linkages.

In some embodiments, the antisense strand of the oligonucleotide described herein comprises a structure of (5′ to 3′; referred to herein as “MAS7”): mN*fN*mNmNmNmNmNmNmNmNmNmNmNfNmNmNmNmNmNmNmN*mN*mN, wherein “mN” indicates 2′-O-methyl (2′-O-Me) modified nucleoside; “fN” indicates 2′-fluoro (2′-F) modified nucleoside; “*” represents phosphorothioate internucleoside linkage; and the absence of “*” between two nucleosides represents phosphodiester internucleoside linkage.

In some embodiments, the MAS7 structure further comprises a 5′ vinylphosphonate (e.g., 5′-(E)-vinylphosphonate) modification (5′ to 3′; referred to herein as “VP-MAS7”): VP-mN*fN*mNmNmNmNmNmNmNmNmNmNmNfNmNmNmNmNmNmNmN*mN*mN, wherein “mN” indicates 2′-O-methyl (2′-O-Me) modified nucleoside; “fN” indicates 2′-fluoro (2′-F) modified nucleoside; “*” represents phosphorothioate internucleoside linkage; the absence of “*” between two nucleosides represents phosphodiester internucleoside linkage; and VP represents 5′-(E)-vinylphosphonate.

In some embodiments, the sense strand of the oligonucleotide described herein comprises a structure of (5′ to 3′; referred to herein as “MS8”): mN*mN*mNmNfNmNmNmNfNfNfNmNmNfNmNmNmNmNfN*mN*mN, wherein “mN” indicates 2′-O-methyl (2′-O-Me) modified nucleoside; “fN” indicates 2′-fluoro (2′-F) modified nucleoside; “*” represents phosphorothioate internucleoside linkage; and the absence of “*” between two nucleosides represents phosphodiester internucleoside linkage.

In some embodiments, the oligonucleotide described herein comprises an antisense strand comprising a structure of (5′ to 3′; VP-MAS7): VP-mN*fN*mNmNmNmNmNmNmNmNmNmNmNfNmNmNmNmNmNmNmN*mN*mN, and a sense strand comprising a structure of (5′ to 3′; MS8): mN*mN*mNmNfNmNmNmNfNfNfNmNmNfNmNmNmNmNfN*mN*mN, wherein “mN” indicates 2′-O-methyl (2′-O-Me) modified nucleoside; “fN” indicates 2′-fluoro (2′-F) modified nucleoside; “*” represents phosphorothioate internucleoside linkage; and the absence of “*” between two nucleosides represents phosphodiester internucleoside linkage; and VP represents 5′-(E)-vinylphosphonate.

In some embodiments, an oligonucleotide described herein (e.g., a DUX4 targeting RNAi oligonucleotide described herein) comprises an antisense strand comprising a nucleobase sequence of SEQ ID NO: 22 and a structure (5′→3′) of VP-mU*fG*mCmCmAmGmAmAmUmUmUmCmAfCmGmGmAmAmGmAmA*mC*mA, and a sense strand comprising a nucleobase sequence of SEQ ID NO: 21 and a structure (5′→3′) of mU*mU*mCmUfUmCmCmGfUfGfAmAmAfUmUmCmUmGfG*mC*mA, wherein: mA, mC, mG, and mU are 2′-O-methyl adenosine, 2′-O-methyl cytidine, 2′-O-methyl guanosine, and 2′-O-methyl uridine, respectively; fA, fC, fG, and fU are 2′-fluoro adenosine, 2′-fluoro cytidine, 2′-fluoro guanosine, and 2′-fluoro uridine, respectively; * between two nucleosides represents a phosphorothioate linkage; the absence of “*” between two nucleosides represents a phosphodiester internucleoside linkage; and “VP” represents 5′-(E)-Vinylphosphonate.

In some embodiments, an oligonucleotide described herein (e.g., a DUX4 targeting RNAi oligonucleotide described herein) comprises a structure of the formula (Ie), in which the antisense strand and the sense strand form a double stranded oligonucleotide:

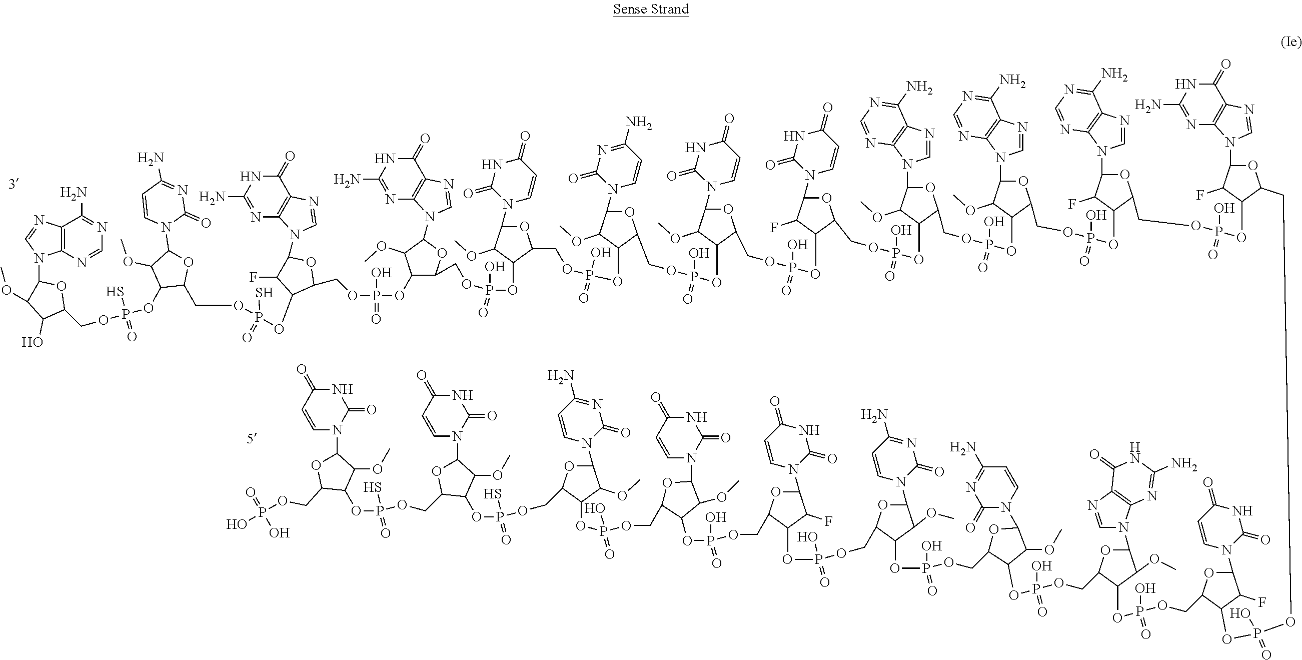

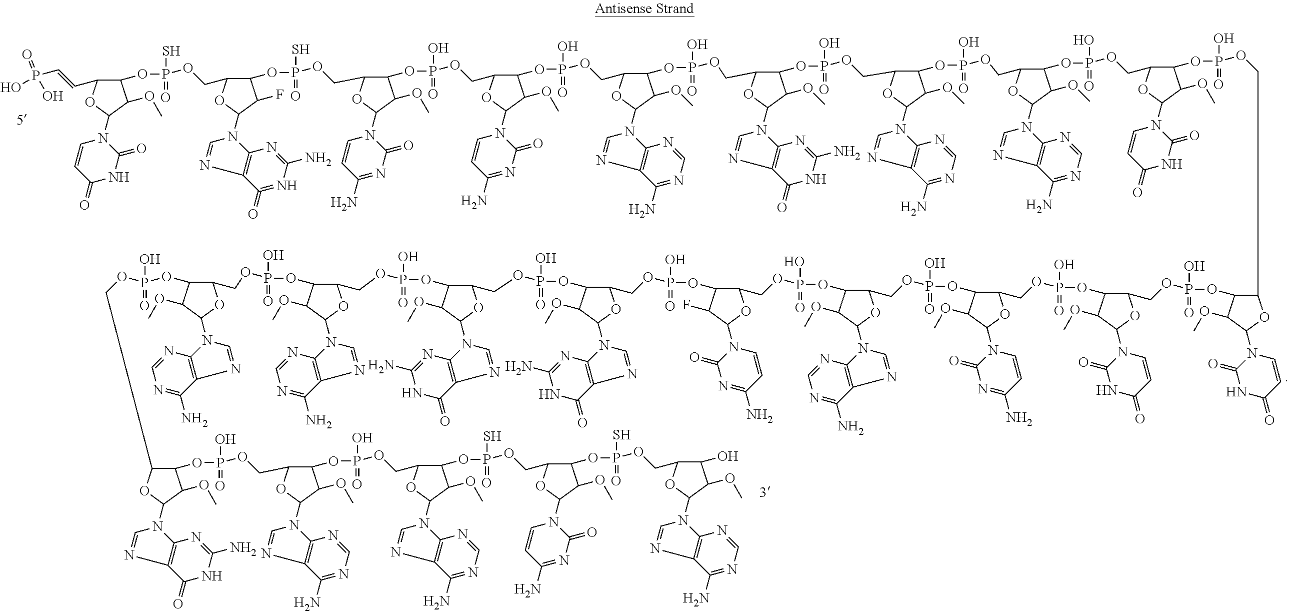

In some embodiments, an oligonucleotide described herein (e.g., a DUX4 targeting RNAi oligonucleotide described herein) can be in salt form, e.g., as sodium, potassium, or magnesium salts.

In some embodiments, the 5′ or 3′ nucleoside (e.g., terminal nucleoside) of an oligonucleotide described herein (e.g., a DUX4-targeting RNAi oligonucleotide) is conjugated to an amine group, optionally via a spacer. In some embodiments, the spacer comprises an aliphatic moiety. In some embodiments, the spacer comprises a polyethylene glycol moiety. In some embodiments, a phosphodiester linkage is present between the spacer and the 5′ or 3′ nucleoside of the oligonucleotide. In some embodiments, the 5′ or 3′ nucleoside (e.g., terminal nucleoside) of an oligonucleotide described herein is covalently linked to a spacer that is a substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, —OC(═O)N(R^{A})—, —S(O)₂NR^{A}—, —NR^{A}S(O)₂—, or a combination thereof; each R^{A} is independently hydrogen or substituted or unsubstituted alkyl. In certain embodiments, the spacer is a substituted or unsubstituted alkylene, substituted or unsubstituted heterocyclylene, substituted or unsubstituted heteroarylene, —O—, —N(R^{A})—, or —C(═O)N(R^{A})₂, or a combination thereof.

In some embodiments, the 5′ or 3′ nucleoside of the oligonucleotide is conjugated to a compound of the formula —NH₂—(CH₂)ₙ—, wherein n is an integer from 1 to 12. In some embodiments, n is 6, 7, 8, 9, 10, 11, or 12. In particular embodiments, n is 6. In some embodiments, a phosphodiester linkage is present between the compound of the formula NH₂—(CH₂)ₙ— and the 5′ or 3′ nucleoside of the oligonucleotide, wherein n is an integer from 1 to 12. In some embodiments, n is 6, 7, 8, 9, 10, 11, or 12. In particular embodiments, n is 6.

In some embodiments, the 5′ nucleoside of the sense strand of the oligonucleotide is conjugated to a compound of the formula —NH₂—(CH₂)ₙ—, wherein n is an integer from 1 to 12 (e.g., 6). In some embodiments, the 3′ nucleoside of the sense strand of the oligonucleotide is conjugated to a compound of the formula —NH₂—(CH₂)ₙ—, wherein n is an integer from 1 to 12 (e.g., 6).

In some embodiments, a compound of the formula NH₂—(CH₂)₆— is conjugated to the oligonucleotide via a reaction between 6-amino-1-hexanol (NH₂—(CH₂)₆—OH) and the 5′ phosphate of the oligonucleotide. In some embodiments, a compound of the formula NH₂—(CH₂)₆— is conjugated to the oligonucleotide via a reaction between 6-amino-1-hexanol (NH₂—(CH₂)₆—OH) and the 5′ phosphate of the sense strand of the oligonucleotide. In some embodiments, a compound of the formula NH₂—(CH₂)₆— is conjugated to the oligonucleotide via a reaction between 6-amino-1-hexanol (NH₂—(CH₂)₆—OH) and the 5′ phosphate of the antisense strand of the oligonucleotide.

In some embodiments, a compound of the formula NH₂—(CH₂)₆— is conjugated to the oligonucleotide via a reaction between 6-amino-1-hexanol (NH₂—(CH₂)₆—OH) and the 3′ phosphate of the oligonucleotide. In some embodiments, a compound of the formula NH₂—(CH₂)₆— is conjugated to the oligonucleotide via a reaction between 6-amino-1-hexanol (NH₂—(CH₂)₆—OH) and the 3′ phosphate of the sense strand of an oligonucleotide. In some embodiments, a compound of the formula NH₂—(CH₂)₆— is conjugated to the oligonucleotide via a reaction between 6-amino-1-hexanol (NH₂—(CH₂)₆—OH) and the 3′ phosphate of the antisense strand of the oligonucleotide.

In some embodiments, an oligonucleotide described herein (e.g., a DUX4 targeting RNAi oligonucleotide described herein) conjugated to the formula NH₂—(CH₂)₆— comprises a structure of the formula (If), in which the antisense strand and the sense strand form a double stranded oligonucleotide:

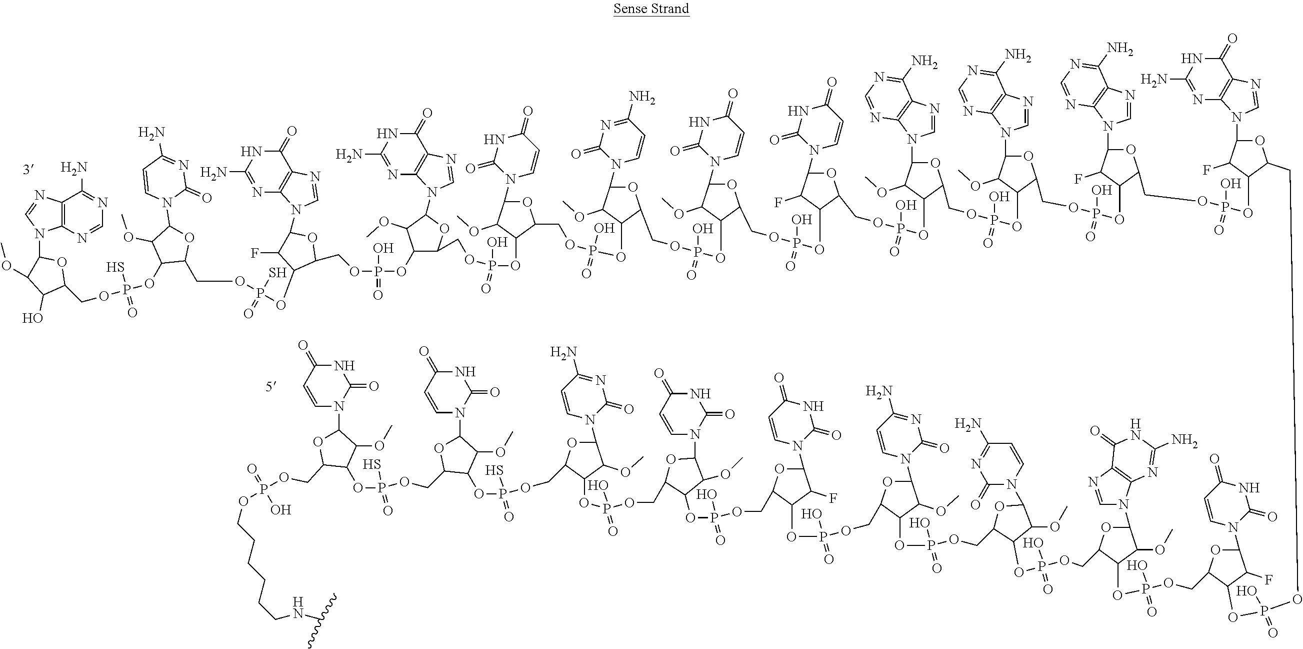

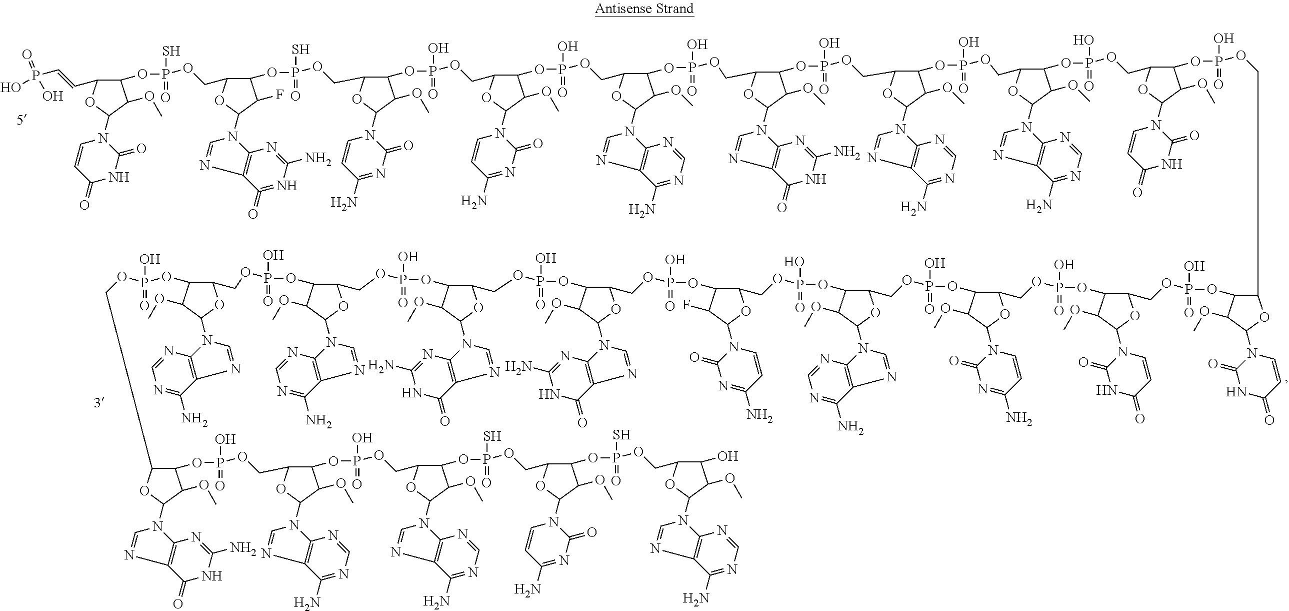

In some embodiments, the oligonucleotide is conjugated to a targeting agent, e.g., a muscle targeting agent such as an anti-TfR1 antibody, e.g., via an amine group of a lysine of the targeting agent.

In some embodiments, it should be appreciated that methylation of the nucleobase uracil at the C5 position forms thymine. Thus, in some embodiments, a nucleotide or nucleoside having a C5 methylated uracil (or 5-methyl-uracil) may be equivalently identified as a thymine nucleotide or nucleoside.

In some embodiments, any one or more of the thymine bases (T's) in any one of the oligonucleotides and/or target sequences provided herein may independently and optionally be uracil bases (U's), and/or any one or more of the U's in any one of the oligonucleotides or target sequences provided herein (e.g., a DUX4-targeting RNAi oligonucleotide) may independently and optionally be T's.

### Compositions

In some embodiments, compositions described herein comprise complexes (i.e., a plurality of complexes), each of which complex comprises an antibody (e.g., anti-TFR1 antibody) covalently linked to one or more oligonucleotides (e.g., a DUX4-targeting RNAi oligonucleotide described herein), wherein the antibody comprises a heavy chain comprising a heavy chain variable region (VH) and a heavy chain constant region, and a light chain comprising a light chain variable region (VL) and a light chain constant region. In some embodiments, the antibody of such complexes comprises a CDR-H1, CDR-H2, CDR-H3, CDR-L1, CDR-L2, and CDR-L3 as set forth in Table 2. Complexes of a composition described herein can comprise any structure provided herein, e.g., a structure of formula (I) (e.g., comprising a group of the formula (Ia), formula (Ib), formula (Ic), formula (Id), or formula (A)).

In some embodiments, compositions described herein comprise complexes (i.e., a plurality of complexes) wherein each complex comprises a structure of formula (I): [R¹]ₙ₁—R², in which each R¹ independently comprises a compound comprising an oligonucleotide (e.g., a DUX4-targeting RNAi oligonucleotide described herein) and is covalently linked to R², wherein R² comprises an antibody (e.g., anti-TfR1 antibody) comprising a heavy chain comprising a heavy chain variable region (VH) and a heavy chain constant region, and a light chain comprising a light chain variable region (VL) and a light chain constant region. In some embodiments, each R¹ of a complex is independently covalently linked to a different amino acid residue (e.g., lysine or cysteine) of R².

In some embodiments, the value of n1 of complexes in the composition is independently and optionally an integer from one up to the number of amino acid residues to which conjugation is desired or targeted (e.g., the number of lysine residues) in the antibody (e.g., an antibody comprised within R²). In some embodiments, the value of n1 of each complex in the composition is independently and optionally selected from 1, 2, 3, 4, 5, 6, 7, 8, 9, 10, 11, 12, 13, 14, 15, 16, 17, 18, 19, 20, 21, 22, 23, 24, 25, 26, and 27. In some embodiments, the value of n1 of each complex in the composition is independently and optionally selected from 1, 2, 3, 4, 5, 6, 7, 8, 9, 10, 11, 12, 13, 14, 15, 16, 17, 18, 19, 20, 21, 22, 23, 24, 25 and 26. In some embodiments, the value of n1 of each complex in the composition is 1. In some embodiments, the value of n1 of each complex in the composition is independently selected and optionally from an integer in the range of 1 to 27, 1 to 26, 1 to 10, 1 to 5, or 1 to 3. In some embodiments, the average value of n1 of complexes of the composition is in the range of 1 to 2, 1 to 3, 1 to 5, 1 to 10, 1 to 26, or 1 to 27. In some embodiments, compositions described herein comprise complexes in which the value of n1 is 0. In some embodiments, the average value of n1 of complexes of the composition is in the range of 0.5 to 5 (e.g., 0.5-5, 1-5, 1-4, 1-3, 3-5, 0.5-4, 0.5-3, 0.5-2, 0.5-1.5, 0.5-1, 0.7-1.5, 1-1.6, 1-1.5, 1-1.4, 1-1.3, 1-1.2, 1.1-1.5, 0.8-2, 0.8-1.5, 0.8-1.3, 0.8-1.2, 0.8-1.1, 0.9-3, 0.9-2, 0.9-1.8, 0.9-1.6, 0.9-1.5, 0.9-1.4, 0.9-1.3, or 0.9-1.2). In some embodiments, the average value of n1 of complexes of the composition is 1.

In some embodiments, a composition described herein comprises an antibody that is not conjugated to an oligonucleotide (e.g., in trace amounts) and an antibody conjugated to one or more oligonucleotides. In some embodiments, the antibody that is not conjugated to an oligonucleotide may be referred to as a compound comprising a structure of formula (I): [R¹]ₙ₁—R², for which n1 is zero. Accordingly, in some embodiments, a composition for administration to a subject in the methods described herein comprises compounds (e.g., complexes) comprising a structure of formula (I): [R¹]ₙ₁—R², for which each R¹ independently comprises a group comprising an oligonucleotide, R² comprises an antibody and n1 is independently an integer of zero or greater that reflects the number of instances of R¹ in each compound (e.g., complex). In some embodiments, the fraction of compounds comprising a structure of formula (I): [R¹]ₙ₁—R², in a composition, for which n1 is zero, compared with all compounds of that structure in the composition for which n1 is one or greater, is less than 10%, less than 5%, less than 1% less than 0.5%, less than 0.1%, less than 0.05%, or less than 0.01%. As such, in some embodiments, the average value of n1 of complexes in a composition disclosed herein is in the range of 0.5 to 5 (e.g., 0.5-5, 1-5, 1-4, 1-3, 3-5, 0.5-4, 0.5-3, 0.5-2, 0.5-1.5, 0.5-1, 0.7-1.5, 1-1.6, 1-1.5, 1-1.4, 1-1.3, 1-1.2, 1.1-1.5, 0.8-2, 0.8-1.5, 0.8-1.3, 0.8-1.2, 0.8-1.1, 0.9-3, 0.9-2, 0.9-1.8, 0.9-1.6, 0.9-1.5, 0.9-1.4, 0.9-1.3, or 0.9-1.2). In some embodiments, the average value of n1 of complexes of the composition is 1.

### Formulations

Complexes provided herein are formulated in a manner suitable for pharmaceutical use. In some embodiments, complexes can be delivered to a subject using a formulation that minimizes degradation, facilitates delivery and/or (e.g., and) uptake, or provides another beneficial property to complexes in the formulation. Accordingly, in some embodiments, it has been found that formulating complexes (e.g., complexes comprising an oligonucleotide covalently linked with a Fab) with tris(hydroxymethyl)aminomethane (also known as tromethamine or THAM) and/or sucrose is particularly advantageous for pharmaceutical use, e.g., as described herein. Thus, in some embodiments, provided herein are formulations (e.g., aqueous solutions, lyophilized forms, or frozen forms) comprising complexes together with tris(hydroxymethyl)aminomethane and/or sucrose. Such formulations can be suitably prepared such that when administered to a subject, either into the immediate environment of a target cell or systemically, a sufficient amount of the complexes enter target muscle cells.

In some embodiments, formulations are provided herein that comprise complexes (i.e., a plurality of complexes) that comprise an oligonucleotide (e.g., an RNAi oligonucleotide) covalently linked to an antibody. In some embodiments, provided herein is a formulation comprising complexes, in which each complex comprises an oligonucleotide (e.g., an RNAi oligonucleotide) covalently linked to an anti-TfR1 antibody, optionally wherein the antibody of such complexes comprises a CDR-H1, CDR-H2, CDR-H3, CDR-L1, CDR-L2, and CDR-L3 as set forth in Table 2, and further, in some embodiments, wherein the complexes are formulated with tris(hydroxymethyl)aminomethane and sucrose. In some embodiments, the antibody is an anti-TfR1 antibody.

In some embodiments, formulations are provided that comprise complexes comprising a structure of formula (I): [R¹]ₙ₁—R², in which each R¹ independently comprises a compound comprising an oligonucleotide (e.g., an RNAi oligonucleotide) and R² comprises an antibody (e.g., anti-TfR1 antibody), and in which n1 is an integer representing the number of instances of R¹ in the complex.

In some embodiments, formulations described herein comprise complexes comprising an antibody that comprises a CDR-H1, CDR-H2, CDR-H3, CDR-L1, CDR-L2, and CDR-L3 as set forth in Table 2. In some embodiments, the antibody comprises a VH comprising the amino acid sequence of SEQ ID NO: 17 and a VL comprising the amino acid sequence of SEQ ID NO: 18. In some embodiments, the antibody is a Fab and comprises a heavy chain comprising the amino acid sequence of SEQ ID NO: 19 and a light chain comprising the amino acid sequence of SEQ ID NO: 20.

In some embodiments, a formulation described herein comprises complexes described herein at a concentration of between 1-200 mg of the complex per mL of the formulation. In some embodiments, a formulation described herein comprises complexes at a concentration of 10-200 mg/mL (e.g., 10-200 mg/mL, 10-100 mg/mL, 10-50 mg/mL, 10-30 mg/mL, 10-20 mg/mL, 15-200 mg/mL, 15-150 mg/mL, 15-100 mg/mL, 15-45 mg/mL, 20-40 mg/mL, 25-35 mg/mL, 25.5-34.5 mg/mL, 26-34 mg/mL, 27-33 mg/mL, 28-32 mg/mL, 29-31 mg/mL, 29.5-30.5 mg/mL, 10-40 mg/mL, 15-35 mg/mL, 20-30 mg/mL, 21-29 mg/mL, 21.2-28.8 mg/mL, 22-28 mg/mL, 23-27 mg/mL, 24-26 mg/mL, 24.5-25.5 mg/mL, 50-200 mg/mL, 100-200 mg/mL, 150-200 mg/mL, 175-200 mg/mL, 20-150 mg/mL, 30-150 mg/mL, 50-150 mg/mL, 100-150 mg/mL, 75-150 mg/mL, or 40-100 mg/mL). In some embodiments, the concentration of complexes in a formulation may vary by up to 20% (e.g., +/−up to 20%, +/−up to 15%, +/−up to 10%, or +/−up to 5%) of a set value.

In some embodiments, any one or a plurality of the complexes described herein is formulated with tris(hydroxymethyl)aminomethane and sucrose in an aqueous solution. In some embodiments, the tris(hydroxymethyl)aminomethane is present in the aqueous solution at a concentration in the range of 5-50 mM (e.g., 5-40 mM, 5-35 mM, 5-30 mM, 10-50 mM, 15-45 mM, 10-40 mM, 20-40 mM, 20-35 mM, 20-30 mM, 21-29 mM, 22-28 mM, 23-27 mM, 24-26 mM). In some embodiments, the tris(hydroxymethyl)aminomethane is present in the aqueous solution at a concentration of approximately 25 mM (e.g., 25 mM). In some embodiments, the sucrose is present in the aqueous solution at a concentration in the range of 2% to 15% weight per volume (w/v %), for example, 3-10 w/v %, 5-10 w/v %, 5-15 w/v %, 7-13 w/v %, 8-15% w/v %, 9-15% w/v %, 9-11% w/v %, 9.5-11% w/v %, or for example, in the range of 9-10 w/v %, 10-11 w/v %, 10-12 w/v %, or 8-12 w/v %. In some embodiments, the sucrose is present in the aqueous solution at a concentration of approximately 3 w/v % (e.g., 3 w/v %). In some embodiments, the sucrose is present in the aqueous solution at a concentration of approximately 6 w/v % (e.g., 6 w/v %). In some embodiments, the sucrose is present in the aqueous solution at a concentration in the range of 7-13 w/v %, 8-12 w/v %, or 9-11 w/v %. In some embodiments, the sucrose is present in the aqueous solution at a concentration of approximately 10 w/v % (e.g., 10 w/v %). In some embodiments, the aqueous solution has a pH in the range of 6.5 to 8.5, for example, 6.5-6.5, 6.7-6.9, 6.9-7.1, 7.1-7.3, 7.2-7.8, 7.3-7.5, 7.4-7.5, 7.4-7.6, 7.5-7.6; for example, 7.0-8.0, or for example, in the pH range of 7.0-7.3, 7.2-7.8, 7.3-7.5, 7.4-7.6, 7.5-7.6, 7.5-7.7, 7.7-7.9, 7.9-8.0, 8.0-8.2, 8.2-8.4, 8.3-8.4, 8.4-8.5, 8.5-8.6, or 7.3-7.7. In some embodiments, the aqueous solution has a pH in the range of 7.0-8.0 (e.g., 7.0-7.8, 7.1-7.8, 7.2-7.8, 7.3-7.7, 7.3-7.5, 7.3-7.6, 7.4-7.6, or 7.4-7.8). In some embodiments, the aqueous solution has a pH of approximately 7.5 (e.g., 7.5). In some embodiments, the aqueous solution has a pH in the range of 7.4-7.7. In some embodiments, the aqueous solution has a pH in the range of 7.4-7.6 (e.g., 7.5, or about 7.5).

In some embodiments, any one of the formulations described herein is an aqueous solution, wherein tris(hydroxymethyl)aminomethane is present in the aqueous solution at a concentration of approximately 25 mM (e.g., 25 mM), wherein sucrose is present in the aqueous solution at a concentration of approximately 3 w/v % (e.g., 3 w/v %), and wherein the aqueous solution is at a pH of approximately 7.5 (e.g., 7.5).

In some embodiments, any one of the formulations described herein is an aqueous solution, wherein tris(hydroxymethyl)aminomethane is present in the aqueous solution at a concentration of approximately 25 mM (e.g., 25 mM), wherein sucrose is present in the aqueous solution at a concentration of approximately 3 w/v % (e.g., 3 w/v %), wherein complexes are present in the aqueous solution at a concentration of approximately 10-200 mg/ml and wherein the aqueous solution is at a pH of approximately 7.5 (e.g., 7.5).

In some embodiments, any one of the formulations described herein is an aqueous solution, wherein tris(hydroxymethyl)aminomethane is present in the aqueous solution at a concentration of approximately 25 mM (e.g., 25 mM), wherein sucrose is present in the aqueous solution at a concentration of approximately 6 w/v % (e.g., 6 w/v %), and wherein the aqueous solution is at a pH of approximately 7.5 (e.g., 7.5).

In some embodiments, any one of the formulations described herein is an aqueous solution, wherein tris(hydroxymethyl)aminomethane is present in the aqueous solution at a concentration of approximately 25 mM (e.g., 25 mM), wherein sucrose is present in the aqueous solution at a concentration of approximately 6 w/v % (e.g., 6 w/v %), wherein complexes are present in the aqueous solution at a concentration of approximately 10-200 mg/ml and wherein the aqueous solution is at a pH of approximately 7.5 (e.g., 7.5).

In some embodiments, any one of the formulations described herein is an aqueous solution, wherein tris(hydroxymethyl)aminomethane is present in the aqueous solution at a concentration of approximately 25 mM (e.g., 25 mM), wherein sucrose is present in the aqueous solution at a concentration of approximately 10 w/v % (e.g., 10 w/v %), and wherein the aqueous solution is at a pH of approximately 7.5 (e.g., 7.5).

In some embodiments, any one of the formulations described herein is an aqueous solution, wherein tris(hydroxymethyl)aminomethane is present in the aqueous solution at a concentration of approximately 25 mM (e.g., 25 mM), wherein sucrose is present in the aqueous solution at a concentration of approximately 10 w/v % (e.g., 10 w/v %), wherein complexes are present in the aqueous solution at a concentration of approximately 10-200 mg/ml and wherein the aqueous solution is at a pH of approximately 7.5 (e.g., 7.5).

In some embodiments, any one or a plurality of the complexes described herein is formulated with tris(hydroxymethyl)aminomethane and sucrose in a lyophilized form (e.g., lyophilized powder). In some embodiments, the lyophilized form (e.g., lyophilized powder) is obtained by lyophilization of any one of the aqueous solutions described herein.

In some embodiments, a lyophilized form is a lyophilized cake. In some embodiments, a lyophilized cake comprises a plurality of complexes provided herein, tris(hydroxymethyl)aminomethane, and sucrose.

In some embodiments, any one or a plurality of the complexes described herein is formulated with tris(hydroxymethyl)aminomethane and sucrose in a frozen form (e.g., a frozen aqueous solid). In some embodiments, the frozen form (e.g., frozen aqueous solid) is obtained by freezing of any one of the aqueous solutions described herein. A frozen form may be frozen to a temperature of less than −20° C. (e.g., less than −20° C., less than −30° C., less than −40° C., less than −50° C., less than −60° C., less than −70° C., less than −80° C., or lower).

In some embodiments, the value of n1 of each complex in the formulation is independently and optionally an integer from zero up to the number of amino acid residues to which conjugation is desired or targeted (e.g., the number of lysine residues) in the antibody (R²). In some embodiments, the value of n1 of each complex in the formulation is independently and optionally selected from 0, 1, 2, 3, 4, 5, 6, 7, 8, 9, 10, 11, 12, 13, 14, 15, 16, 17, 18, 19, 20, 21, 22, 23, 24, 25, 26, and 27. In some embodiments, the value of n1 of each complex in the formulation is independently and optionally selected from 0, 1, 2, 3, 4, 5, 6, 7, 8, 9, 10, 11, 12, 13, 14, 15, 16, 17, 18, 19, 20, 21, 22, 23, 24, 25 and 26. In some embodiments, the value of n1 of each complex in the formulation is 1. In some embodiments, the value of n1 of each complex in the formulation is independently selected and optionally from an integer in the range of 1 to 27, 1 to 26, 1 to 10, 1 to 5, or 1 to 3. In some embodiments, the average value of n1 of complexes in the formulation is in the range of 1 to 2, 1 to 3, 1 to 5, 1 to 10, 1 to 26 or 1 to 27. In some embodiments, formulations described herein comprise complexes that comprise a structure of formula (I): [R¹]ₙ₁—R², wherein n1 is 0. In some embodiments, the average value of n1 of complexes of the formulation is in the range of 0.5 to 5 (e.g., 0.5-5, 1-5, 1-4, 1-3, 3-5, 0.5-4, 0.5-3, 0.5-2, 0.5-1.5, 0.5-1, 0.7-1.5, 1-1.6, 1-1.5, 1-1.4, 1-1.3, 1-1.2, 1.1-1.5, 0.8-2, 0.8-1.5, 0.8-1.3, 0.8-1.2, 0.8-1.1, 0.9-3, 0.9-2, 0.9-1.8, 0.9-1.6, 0.9-1.5, 0.9-1.4, 0.9-1.3, or 0.9-1.2). In some embodiments, the average value of n1 of complexes of the formulation is 1.

In some embodiments, each instance of R¹ in a complex herein (e.g., a complex of a formulation provided herein) is conjugated to a different amino acid residue of the antibody. In some embodiments, each different amino acid comprises an ε-amino group (e.g., lysine, arginine). However, in some embodiments, each different amino acid to which R¹ is covalently linked is a cysteine. In some embodiments, R¹ is directly covalently linked to an amino acid residue of the antibody. However, in some embodiments, R¹ is indirectly covalently linked to an amino acid of the antibody, e.g., covalently linked to a glycosylation site on the amino acid. In some embodiments, formulations are provided in which complexes for which R¹ is covalently linked to an amino acid residue residing in a CDR region of the antibody are present in only trace amounts, or in undetectable amount, or not at all. In some embodiments, formulations are provided in which complexes for which R¹ is covalently linked to an amino acid residue residing in a CDR region of the antibody are not detectable in the formulation using standard detection techniques.

In some embodiments, formulations provided herein comprise complexes that comprise a structure of formula (I): [R¹]ₙ₁—R², in which each instance of R¹ independently comprises a group of the formula (Ia):

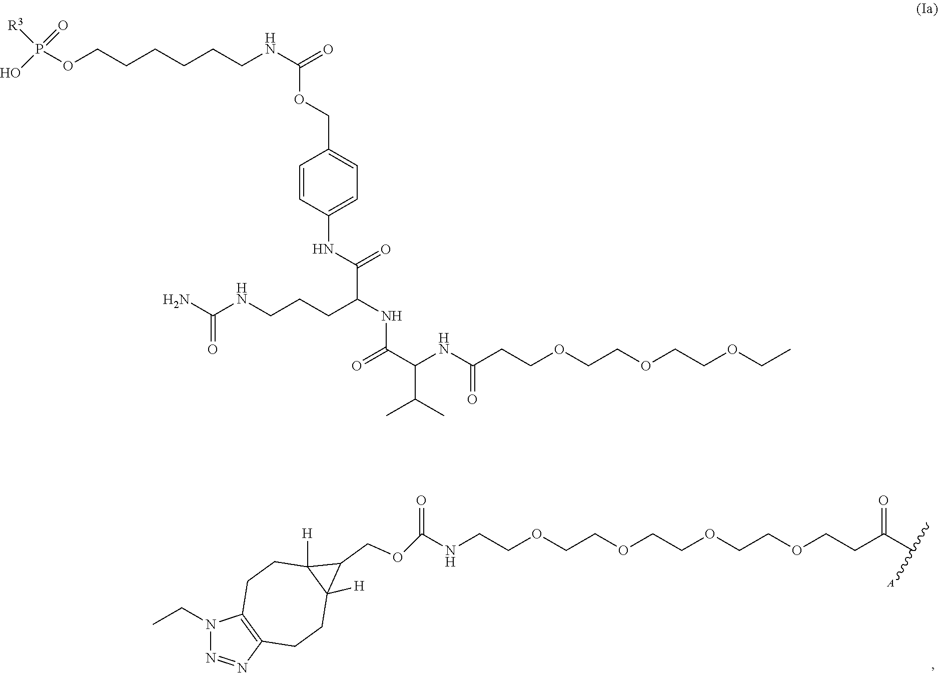

or a pharmaceutically acceptable salt thereof,
in which R³ comprises an oligonucleotide, e.g., an RNAi oligonucleotide; and R¹ is covalently linked to R² at attachment point A. In some embodiments, R² comprises an antibody comprising a sequence as set forth in Table 2. For example, in some embodiments, R² comprises an antibody comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5, or 11, and a light chain complementarity determining region 3 (CDR-L3) comprising a sequence as set forth in SEQ ID NO: 6 or 16. In some embodiments, R² comprises an antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody comprising a VH comprising the amino acid sequence of SEQ ID NO: 17 and/or comprising a VL comprising the amino acid sequence of SEQ ID NO: 18. In some embodiments, R² comprises an antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)₂ fragment, an scFv, or an Fv. In some embodiments, R² comprises an antibody that is a Fab fragment. In some embodiments, R³ comprises an RNAi oligonucleotide comprising an antisense strand comprising a nucleobase sequence of SEQ ID NO: 22 and a structure (5′→3′) of VP-mU*fG*mCmCmAmGmAmAmUmUmUmCmAfCmGmGmAmAmGmAmA*mC*mA, and a sense strand comprising a nucleobase sequence of SEQ ID NO: 21 and a structure (5′→3′) of mU*mU*mCmUfUmCmCmGfUfGfAmAmAfUmUmCmUmGfG*mC*mA, wherein: mA, mC, mG, and mU are 2′-O-methyl adenosine, 2′-O-methyl cytidine, 2′-O-methyl guanosine, and 2′-O-methyl uridine, respectively; fA, fC, fG, and fU are 2′-fluoro adenosine, 2′-fluoro cytidine, 2′-fluoro guanosine, and 2′-fluoro uridine, respectively; * between two nucleosides represents a phosphorothioate linkage; the absence of “*” between two nucleosides represents a phosphodiester internucleoside linkage; and “VP” represents 5′-(E)-Vinylphosphonate. In some embodiments, in each complex n1 is independently an integer (e.g., an integer in the range of 1-27, 1-26, 1-10, 1-5, or 1-3). In some embodiments, in each complex n1 is 1. In some embodiments, formulations described herein further comprise complexes that comprise a structure of formula (I): [R¹]ₙ₁—R², wherein n1 is 0. In some embodiments, the average value of n1 of complexes of the composition is in the range of 0.5 to 5 (e.g., 0.5-5, 1-5, 1-4, 1-3, 3-5, 0.5-4, 0.5-3, 0.5-2, 0.5-1.5, 0.5-1, 0.7-1.5, 1-1.6, 1-1.5, 1-1.4, 1-1.3, 1-1.2, 1.1-1.5, 0.8-2, 0.8-1.5, 0.8-1.3, 0.8-1.2, 0.8-1.1, 0.9-3, 0.9-2, 0.9-1.8, 0.9-1.6, 0.9-1.5, 0.9-1.4, 0.9-1.3, or 0.9-1.2). In some embodiments, the average value of n1 of complexes of the composition is 1. In some embodiments, formulations further comprise tris(hydroxymethyl)aminomethane and sucrose. In some embodiments, formulations comprise tris(hydroxymethyl)aminomethane at a concentration of 25 mM and/or (e.g., and) sucrose at a concentration of 10 w/v %, optionally wherein the formulation has a pH of 7.5. In some embodiments, formulations comprise complexes at a concentration of 30 mg/mL.

In some embodiments, formulations provided herein comprise complexes that comprise a structure of formula (I): [R¹]ₙ₁—R², in which each instance of R¹ in a complex of a formulation provided herein comprises a group of the formula (Ib):

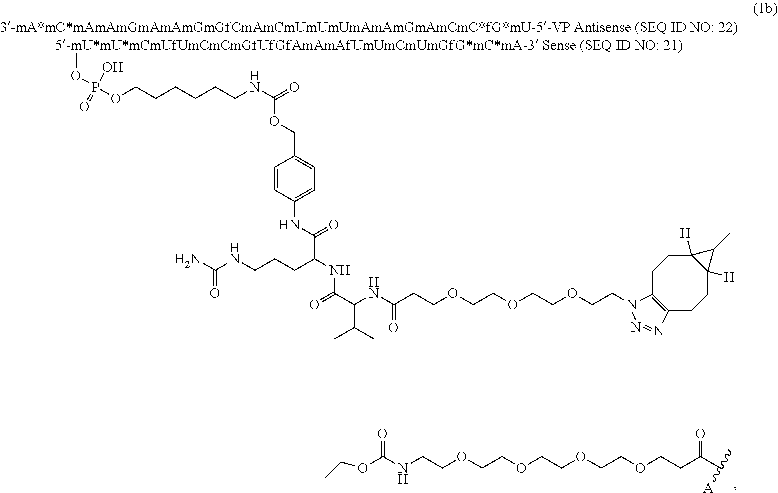

or a pharmaceutically acceptable salt thereof,
wherein: mA, mC, mG, and mU are 2′-O-methyl adenosine, 2′-O-methyl cytidine, 2′-O-methyl guanosine, and 2′-O-methyl uridine, respectively; fA, fC, fG, and fU are 2′-fluoro adenosine, 2′-fluoro cytidine, 2′-fluoro guanosine, and 2′-fluoro uridine, respectively; * between two nucleosides represents a phosphorothioate linkage; the absence of “*” between two nucleosides represents a phosphodiester internucleoside linkage; and “VP” represents 5′-(E)-Vinylphosphonate; and wherein the oligonucleotide is an RNAi oligonucleotide (e.g., siRNA) comprising an antisense strand comprising a nucleobase sequence of UGCCAGAAUUUCACGGAAGAACA (SEQ ID NO: 22) and a sense strand comprising a nucleobase sequence of UUCUUCCGUGAAAUUCUGGCA (SEQ ID NO: 21); wherein n1 is an integer (e.g., one or greater) representing the number of instances of R¹ in each complex, and each R¹ is covalently linked to R² at attachment point A. In some embodiments, R² comprises an antibody comprising a sequence as set forth in Table 2. For example, in some embodiments, R² comprises an antibody comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5, or 11, and a light chain complementarity determining region 3 (CDR-L3) comprising a sequence as set forth in SEQ ID NO: 6 or 16. In some embodiments, R² comprises an antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody comprising a VH comprising the amino acid sequence of SEQ ID NO: 17 and/or comprising a VL comprising the amino acid sequence of SEQ ID NO: 18. In some embodiments, R² comprises an antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)₂ fragment, an scFv, or an Fv. In some embodiments, R² comprises an antibody that is a Fab fragment. In some embodiments, formulations described herein further comprise complexes that comprise a structure of formula (I): [R¹]ₙ₁—R², wherein n1 is 0. In some embodiments, the average value of n1 of complexes of the formulation is in the range of 0.5 to 5 (e.g., 0.5-5, 1-5, 1-4, 1-3, 3-5, 0.5-4, 0.5-3, 0.5-2, 0.5-1.5, 0.5-1, 0.7-1.5, 1-1.6, 1-1.5, 1-1.4, 1-1.3, 1-1.2, 1.1-1.5, 0.8-2, 0.8-1.5, 0.8-1.3, 0.8-1.2, 0.8-1.1, 0.9-3, 0.9-2, 0.9-1.8, 0.9-1.6, 0.9-1.5, 0.9-1.4, 0.9-1.3, or 0.9-1.2). In some embodiments, the average value of n1 of complexes of the formulation is 1. In some embodiments, formulations further comprise tris(hydroxymethyl)aminomethane and sucrose. In some embodiments, formulations comprise tris(hydroxymethyl)aminomethane at a concentration of 25 mM and/or (e.g., and) sucrose at a concentration of 10 w/v %, optionally wherein the formulation has a pH of 7.5. In some embodiments, formulations comprise complexes at a concentration of 30 mg/mL.

In some embodiments, formulations provided herein comprise complexes that comprise a structure of formula (I): [R¹]ₙ₁—R², in which each R¹ comprises a group of the formula (Ic), in which the antisense strand and the sense strand form a double stranded oligonucleotide:

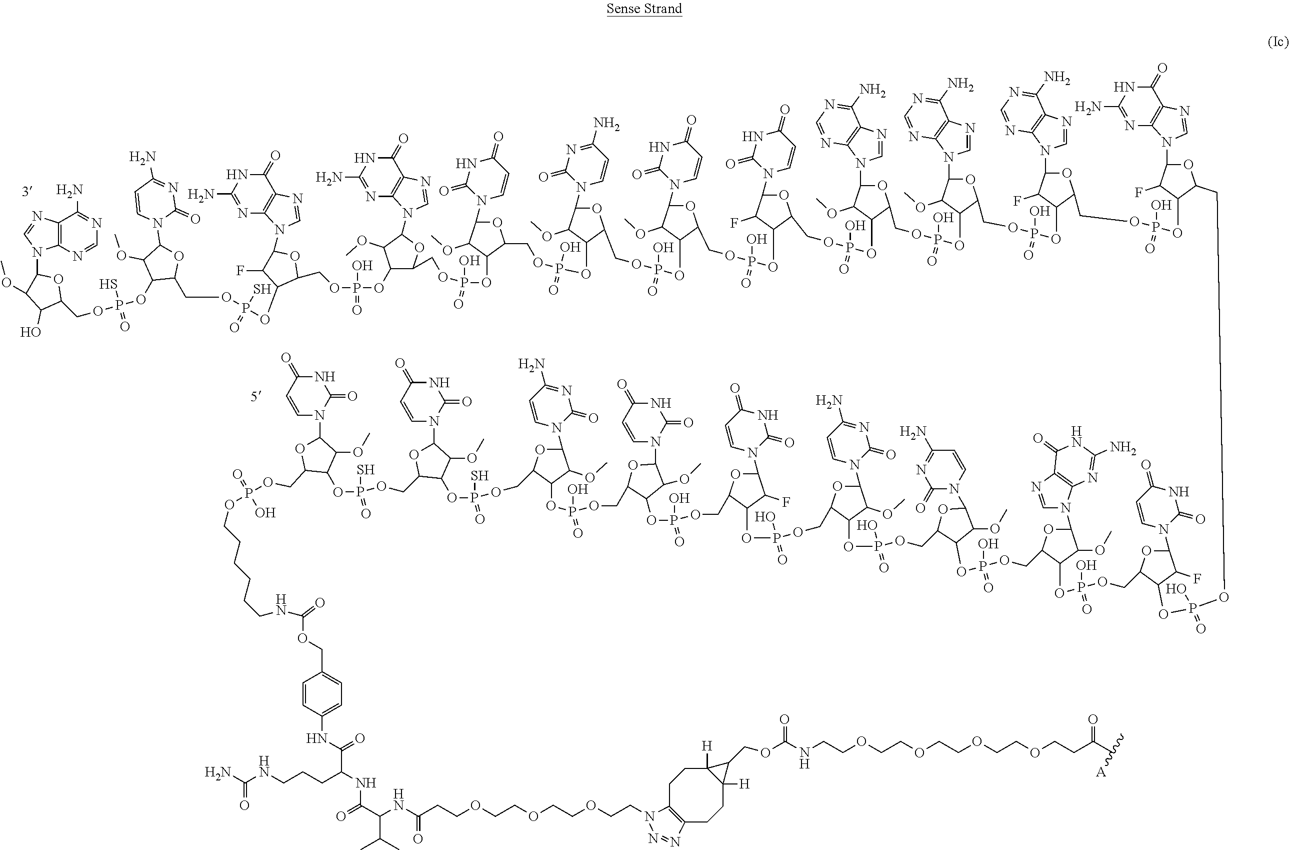

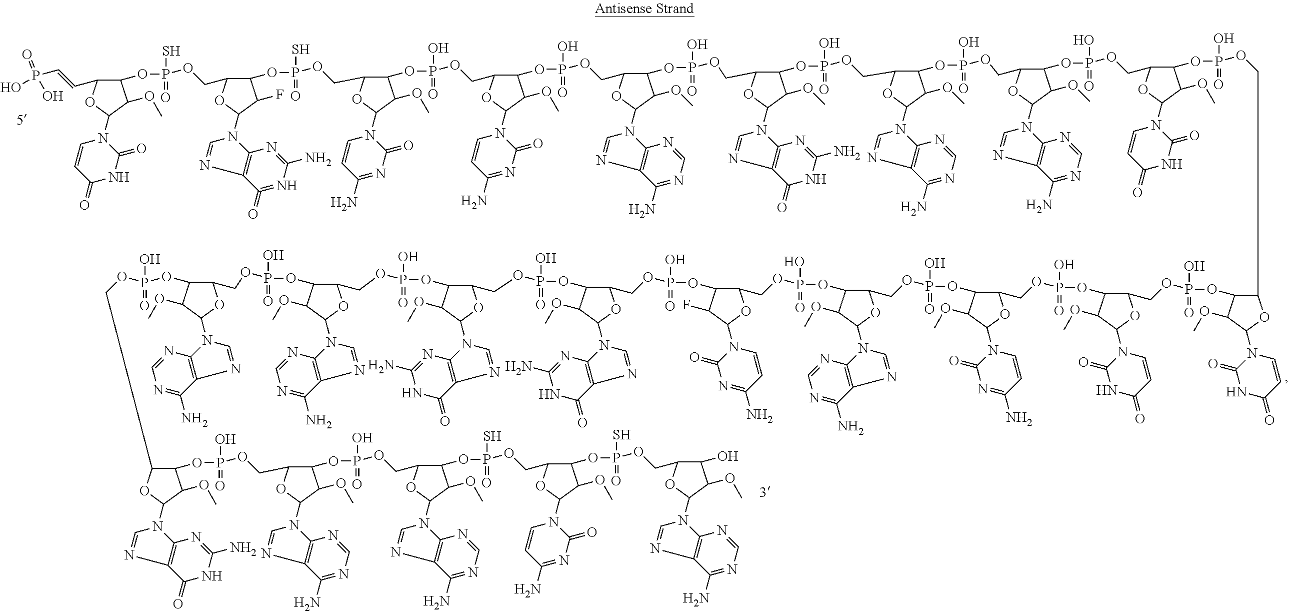

or a pharmaceutically acceptable salt thereof,
wherein R¹ is covalently linked to R² at attachment point A. In some embodiments, n1 is an integer (e.g., one or greater) representing the number of instances of R¹ in each complex. In some embodiments, R² comprises an antibody comprising a sequence as set forth in Table 2. For example, in some embodiments, R² comprises an antibody comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5, or 11, and a light chain complementarity determining region 3 (CDR-L3) comprising a sequence as set forth in SEQ ID NO: 6 or 16. In some embodiments, R² comprises an antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody comprising a VH comprising the amino acid sequence of SEQ ID NO: 17 and/or comprising a VL comprising the amino acid sequence of SEQ ID NO: 18. In some embodiments, R² comprises an antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)₂ fragment, an scFv, or an Fv. In some embodiments, R² comprises an antibody that is a Fab fragment. In some embodiments, formulations described herein further comprise complexes that comprise a structure of formula (I): [R¹]ₙ₁—R², wherein n1 is 0. In some embodiments, the average value of n1 of complexes of the formulation is in the range of 0.5 to 5 (e.g., 0.5-5, 1-5, 1-4, 1-3, 3-5, 0.5-4, 0.5-3, 0.5-2, 0.5-1.5, 0.5-1, 0.7-1.5, 1-1.6, 1-1.5, 1-1.4, 1-1.3, 1-1.2, 1.1-1.5, 0.8-2, 0.8-1.5, 0.8-1.3, 0.8-1.2, 0.8-1.1, 0.9-3, 0.9-2, 0.9-1.8, 0.9-1.6, 0.9-1.5, 0.9-1.4, 0.9-1.3, or 0.9-1.2). In some embodiments, the average value of n1 of complexes of the formulation is 1. In some embodiments, formulations further comprise tris(hydroxymethyl)aminomethane and sucrose. In some embodiments, formulations comprise tris(hydroxymethyl)aminomethane at a concentration of 25 mM and/or (e.g., and) sucrose at a concentration of 10 w/v %, optionally wherein the formulation has a pH of 7.5. In some embodiments, formulations comprise complexes at a concentration of 30 mg/mL.

In some embodiments, formulations provided herein comprise complexes that comprise a structure of the formula (Id):

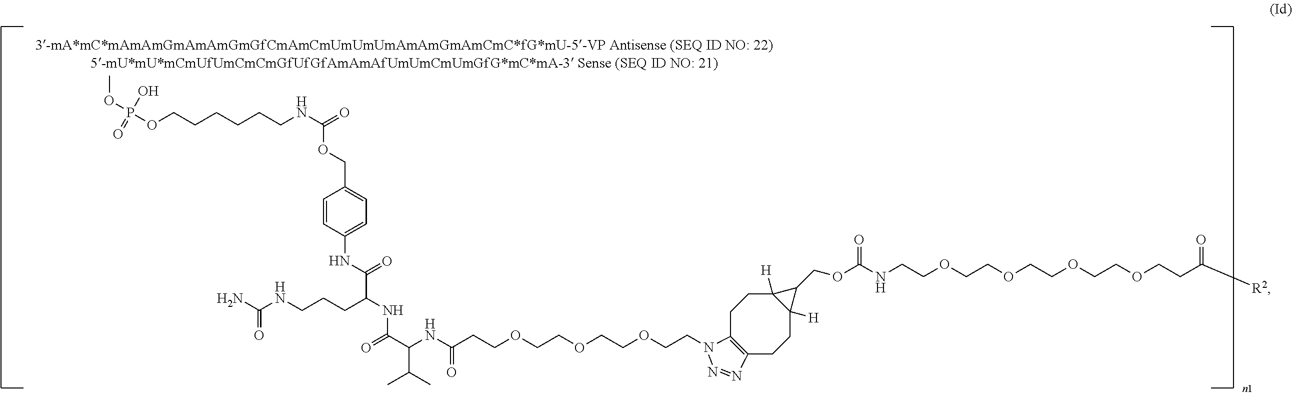

or a pharmaceutically acceptable salt thereof,
wherein: mA, mC, mG, and mU are 2′-O-methyl adenosine, 2′-O-methyl cytidine, 2′-O-methyl guanosine, and 2′-O-methyl uridine, respectively; fA, fC, fG, and fU are 2′-fluoro adenosine, 2′-fluoro cytidine, 2′-fluoro guanosine, and 2′-fluoro uridine, respectively; * between two nucleosides represents a phosphorothioate linkage; the absence of “*” between two nucleosides represents a phosphodiester internucleoside linkage; and “VP” represents 5′-(E)-Vinylphosphonate; and wherein the oligonucleotide is an RNAi oligonucleotide (e.g., siRNA) comprising an antisense strand comprising a nucleobase sequence of UGCCAGAAUUUCACGGAAGAACA (SEQ ID NO: 22) and a sense strand comprising a nucleobase sequence of UUCUUCCGUGAAAUUCUGGCA (SEQ ID NO: 21); wherein R² comprises an antibody comprising a sequence as set forth in Table 2; wherein n1 is an integer (e.g., one or greater) representing the number of instances of the group enclosed by square brackets, wherein each instance of the group enclosed by square brackets is covalently linked to a different amino acid residue of the antibody, optionally wherein each different amino acid residue is a lysine. In some embodiments, R² comprises an antibody comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5, or 11, and a light chain complementarity determining region 3 (CDR-L3) comprising a sequence as set forth in SEQ ID NO: 6 or 16. In some embodiments, R² comprises an antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody comprising a VH comprising the amino acid sequence of SEQ ID NO: 17 and/or comprising a VL comprising the amino acid sequence of SEQ ID NO: 18. In some embodiments, R² comprises an antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)₂ fragment, an scFv, or an Fv. In some embodiments, R² comprises an antibody that is a Fab fragment. In some embodiments, formulations described herein further comprise complexes in which n1 is 0. In some embodiments, the average value of n1 of complexes of the formulation is in the range of 0.5 to 5 (e.g., 0.5-5, 1-5, 1-4, 1-3, 3-5, 0.5-4, 0.5-3, 0.5-2, 0.5-1.5, 0.5-1, 0.7-1.5, 1-1.6, 1-1.5, 1-1.4, 1-1.3, 1-1.2, 1.1-1.5, 0.8-2, 0.8-1.5, 0.8-1.3, 0.8-1.2, 0.8-1.1, 0.9-3, 0.9-2, 0.9-1.8, 0.9-1.6, 0.9-1.5, 0.9-1.4, 0.9-1.3, or 0.9-1.2). In some embodiments, the average value of n1 of complexes of the formulation is 1. In some embodiments, formulations further comprise tris(hydroxymethyl)aminomethane and sucrose. In some embodiments, formulations comprise tris(hydroxymethyl)aminomethane at a concentration of 25 mM and/or (e.g., and) sucrose at a concentration of 10 w/v %, optionally wherein the formulation has a pH of 7.5. In some embodiments, formulations comprise complexes at a concentration of 30 mg/mL.

In some embodiments, complexes provided in the formulations described herein comprise a structure of formula (A):

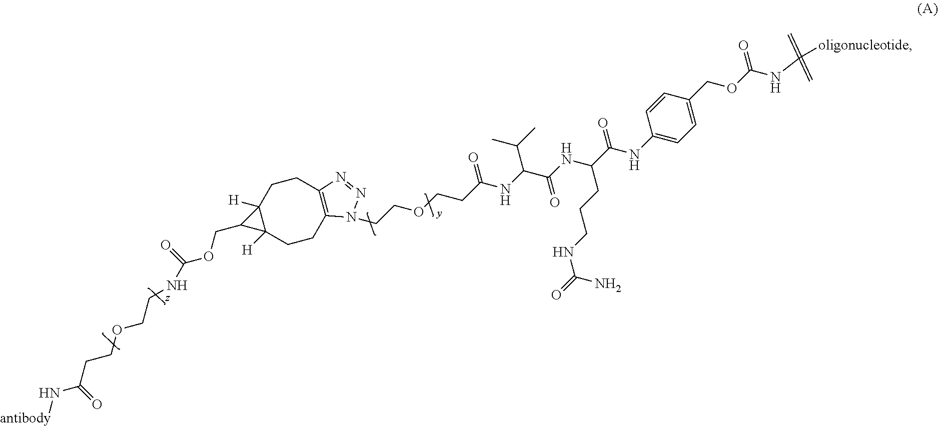

wherein y is 0-15 (e.g., 3) and z is 0-15 (e.g., 4), and wherein

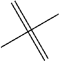

represents an indirect or direct linkage. In some embodiments, the amide shown adjacent the antibody (e.g., anti-TfR1 antibody) in the structure (A) results from a reaction with an amine of the antibody, such as a lysine epsilon amine. In some embodiments, a complex described herein comprises an anti-TfR1 antibody (e.g., an anti-TfR1 Fab) covalently linked via a lysine of the antibody to the 5′ end of an oligonucleotide (e.g., an RNAi oligonucleotide). In some embodiments, the antibody comprises a sequence as set forth in Table 2. For example, in some embodiments, the antibody comprises a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprises a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5, or 11, and a light chain complementarity determining region 3 (CDR-L3) comprising a sequence as set forth in SEQ ID NO: 6 or 16. In some embodiments, the antibody comprises a heavy chain variable region (VH) comprising an amino acid sequence at least 85% (e.g., at least 95%) identical to SEQ ID NO: 17 and/or comprises a light chain variable region (VL) comprising an amino acid sequence at least 85% (e.g., at least 95%) identical to SEQ ID NO: 18. In some embodiments, the antibody comprises a VH comprising the amino acid sequence of SEQ ID NO: 17 and/or comprises a VL comprising the amino acid sequence of SEQ ID NO: 18. In some embodiments, the antibody comprises a heavy chain comprising an amino acid sequence at least 85% (e.g., at least 95%) identical to SEQ ID NO: 19 and/or comprises a light chain comprising an amino acid sequence at least 85% (e.g., at least 95%) identical to SEQ ID NO: 20. In some embodiments, the antibody comprises a heavy chain comprising the amino acid sequence of SEQ ID NO: 19 and/or comprises a light chain comprising the amino acid sequence of SEQ ID NO: 20. In some embodiments, the antibody is a Fab fragment, a full-length IgG, a Fab′ fragment, a F(ab′)₂ fragment, an scFv, or an Fv. In some embodiments, the antibody is a Fab fragment. In some embodiments, formulations further comprise tris(hydroxymethyl)aminomethane and sucrose. In some embodiments, formulations comprise tris(hydroxymethyl)aminomethane at a concentration of 25 mM and/or (e.g., and) sucrose at a concentration of 10 w/v %, optionally wherein the formulation has a pH of 7.5. In some embodiments, formulations comprise complexes at a concentration of 30 mg/mL.

As described herein, in some embodiments, formulations provided herein comprise sucrose. In some embodiments, sucrose serves at least in part as a lyoprotectant. In some embodiments, the sucrose is from a plant, e.g., grass, fruit, or vegetable (e.g., root vegetable) source (e.g., beet (e.g., sugar beet, for example, Saccharum spp.)), sugarcane (e.g., Beta vulgaris), dates, sugar maple, sweet sorghum, apples, oranges, carrots, molasses, maple syrup, corn sweeteners) or animal product (e.g., honey). In some embodiments, the sucrose is from beet or sugarcane (e.g., beet sucrose, sugarcane sucrose). In some embodiments, a lyoprotectant other than sucrose may be used, e.g., trehalose, mannitol, lactose, polyethylene glycol, or polyvinyl pyrrolidone. However, in some embodiments, a collapse temperature modifier (e.g., dextran, ficoll, or gelatin) may be provided in a formulation.

In some embodiments, provided is a product (e.g., lyophilized formulation described herein), produced by a process comprising lyophilizing an aqueous solution of a formulation (e.g., aqueous form) described herein.

In some embodiments, a pharmaceutical composition is formulated to be compatible with its intended route of administration. Examples of routes of administration include parenteral, e.g., intravenous, intradermal, subcutaneous, administration. Typically, the route of administration is intravenous or subcutaneous.

### Methods of Use/Treatment

Complexes comprising a muscle-targeting agent (e.g., an anti-TfR1 antibody) covalently linked to a molecular payload (e.g., an RNAi oligonucleotide) as described herein are effective in treating FSHD. In some embodiments, complexes are effective in treating Type 1 FSHD. In some embodiments, complexes are effective in treating Type 2 FSHD. In some embodiments, FSHD is associated with deletions in D4Z4 repeat regions on chromosome 4 which contain the DUX4 gene. In some embodiments, FSHD is associated with mutations in the SMCHD1 gene. In some embodiments, FSHD is associated with mutations in the DNMT3B gene. In some embodiments, FSHD is associated with mutations in the LRIF1 gene. In some embodiments, a subject may be a human subject, a non-human primate subject, a rodent subject, or any suitable mammalian subject. In some embodiments, a subject may have myotonic dystrophy. In some embodiments, a subject has elevated expression of the DUX4 gene outside of fetal development and the testes. In some embodiments, the subject has facioscapulohumeral muscular dystrophy of Type 1 or Type 2. In some embodiments, the subject having FSHD has mutations in the SMCHD1 gene. In some embodiments, the subject having FSHD has mutations in the DNMT3B gene. In some embodiments, the subject having FSHD has mutations in the LRIF1 gene. In some embodiments, the subject having FSHD has deletion mutations in D4Z4 repeat regions on chromosome 4.

An aspect of the disclosure includes methods involving administering to a subject a composition (e.g., a formulation) comprising an effective amount of complex(es) as described herein. In some embodiments, an effective amount of a pharmaceutical composition that comprises complex(es) comprising an antibody (e.g., Fab) described herein covalently linked to an oligonucleotide (e.g., a DUX4 targeting RNAi oligonucleotide) described herein can be administered to a subject in need of treatment. In some embodiments, a pharmaceutical composition comprising complex(es) as described herein may be administered by a suitable route, which may include intravenous administration, e.g., as a bolus or by continuous infusion over a period of time. In some embodiments, a pharmaceutical composition comprising complex(es) as described herein may be administered by a suitable route, which may include subcutaneous administration. In some embodiments, administration may be performed by intravenous, intramuscular, intraperitoneal, intracerebrospinal, subcutaneous, intra-articular, intrasynovial, or intrathecal routes. In some embodiments, a pharmaceutical composition may be in solid form, aqueous form, or a liquid form. In some embodiments, an aqueous or liquid form may be nebulized or lyophilized. In some embodiments, a lyophilized form may be reconstituted with an aqueous or liquid solution.

In some embodiments, provided are methods of and/or uses for treating a subject having aberrant (e.g., increased) expression of DUX4 RNA and/or protein in muscle comprising administering to the subject a composition (e.g., a formulation) described herein that comprises an effective amount of complex(es) described herein. In some embodiments, provided are methods of and/or uses for reducing the expression or activity of DUX4 (e.g., reducing levels of DUX4 RNA and/or protein) in a cell (e.g., a muscle cell), the methods comprising contacting the cell with a formulation described herein comprising an effective amount of complex(es) described herein. In some embodiments, the method comprises administering a lyophilized form (e.g., lyophilized powder) of the formulation described herein, comprising reconstituting a lyophilized form of the formulation in an aqueous solution, and administering the aqueous solution of the formulation to a subject in need thereof. For example, in some embodiments, a lyophilized form of the formulation is shipped and/or stored in the lyophilized form, reconstituted at a location for administering the aqueous solution of the formulation (e.g., healthcare provider location), and administered in the reconstituted form (e.g., as an aqueous solution) by injection or intravenously, e.g., by infusion.

In some embodiments, a pharmaceutical composition is administered via site-specific or local delivery techniques. Examples of these techniques include implantable depot sources of the complex, local delivery catheters, site specific carriers, direct injection, or direct application.

In some embodiments, a pharmaceutical composition that comprises a complex comprising a an anti-TfR1 antibody (e.g., a fab) covalently linked to a molecular payload (e.g., a DUX4 targeting RNAi oligonucleotide) is administered at an effective concentration that confers therapeutic effect on a subject. Effective amounts vary, as recognized by those skilled in the art, depending on the severity of the disease, unique characteristics of the subject being treated, e.g. age, physical conditions, health, or weight, the duration of the treatment, the nature of any concurrent therapies, the route of administration and related factors. These related factors are known to those in the art and may be addressed with no more than routine experimentation. In some embodiments, an effective concentration is the maximum dose that is considered to be safe for the patient. In some embodiments, an effective concentration will be the lowest possible concentration that provides maximum efficacy.

Empirical considerations, e.g. the half-life of the complex(es) in a subject, generally will contribute to determination of the concentration of pharmaceutical composition that is used for treatment. The frequency of administration may be empirically determined and adjusted to maximize the efficacy of the treatment.

The efficacy of treatment may be assessed using any suitable methods. In some embodiments, the efficacy of treatment may be assessed by evaluation of observation of symptoms associated with FSHD including muscle mass loss and muscle atrophy, primarily in the muscles of the face, shoulder blades, and upper arms.

In some embodiments, a pharmaceutical composition that comprises a complex comprising a muscle-targeting agent (e.g., an anti-TfR1 antibody) covalently linked to a molecular payload (e.g., an RNAi oligonucleotide) described herein is administered to a subject at an effective concentration sufficient to modulate activity or expression of a target gene by at least 10%, at least 20%, at least 30%, at least 40%, at least 50%, at least 60%, at least 70%, at least 80%, at least 90% or at least 95% relative to a control, e.g. baseline level of gene expression prior to treatment.

### EXAMPLES

### Example 1: Activities of Anti-TfR1 Fab-siRNA Complexes in FSHD Patient Myotubes

Activities of complexes containing an anti-TfR1 antibody provided in Table 2 covalently linked via a linker to a DUX4-targeting siRNA were tested in AB1080 immortalized FSHD patient-derived myotubes. The DUX4-targeting siRNA comprises an antisense strand comprising a nucleobase sequence of SEQ ID NO: 22 and a structure (5′→3′) of VP-mU*fG*mCmCmAmGmAmAmUmUmUmCmAfCmGmGmAmAmGmAmA*mC*mA and a sense strand comprising a nucleobase sequence of SEQ ID NO: 21 and a structure (5′→3′) of mU*mU*mCmUfUmCmCmGfUfGfAmAmAfUmUmCmUmGfG*mC*mA, wherein: mA, mC, mG, and mU are 2′-O-methyl adenosine, 2′-O-methyl cytidine, 2′-O-methyl guanosine, and 2′-O-methyl uridine, respectively; fA, fC, fG, and fU are 2′-fluoro adenosine, 2′-fluoro cytidine, 2′-fluoro guanosine, and 2′-fluoro uridine, respectively; * between two nucleosides represents a phosphorothioate linkage; the absence of “*” between two nucleosides represents a phosphodiester internucleoside linkage; and “VP” represents 5′-(E)-Vinylphosphonate.

Two versions of the anti-TfR1 Fab-siRNA complex were tested, one in which the anti-TfR1 Fab was covalently linked to the 3′ end of the sense strand of the siRNA and one in which the anti-TfR1 Fab was covalently linked to the 5′ end of the sense strand of each siRNA. A complex containing an anti-TfR1 antibody provided in Table 2 covalently linked via a linker to an exon 1 targeting siRNA was used as a positive control.

AB1080 (C6) immortalized FSHD patient-derived myotubes were treated with the siRNA complexes at a concentration equivalent to 10 nM, 100 nM, or 1000 nM for 10 days. cDNA was obtained from cells with the TaqMan Fast Advanced Cells-to-Ct Kit (Thermo Fisher Scientific), and levels of three DUX4 transcriptome markers MBD3L2 (Hs00544743_m1), TRIM43 (Hs00299174_m1), ZSCAN4 (Hs00537549_m1), and RPL13A(Hs04194366_g1) were analyzed via qPCR with specific TaqMan assays (Thermo Fisher Scientific). Two-step amplification reactions and fluorescence measurements for determination of cycle threshold (Ct) were conducted on a QuantStudio 7 instrument (Thermo Fisher Scientific). Composite scores calculated using the average mRNA level of three DUX4 transcriptome markers MBD3L2, TRIM43, and ZSCAN4 in FSHD patient-derived C6 myotubes are shown in FIG. 1.

Results show that the tested anti-TfR1 Fab-siRNA complexes achieved significant level of knockdown of the tested DUX4 transcriptome markers in FSHD patient-derived C6 myotubes compared to the positive control.

### Example 2: Activities of an anti-TfR1 Fab-siRNA Complex in FSHD Patient-Derived Primary Cells

Activities of a complex containing an anti-TfR1 antibody provided in Table 2 covalently linked to a DUX4-targeting siRNA, were tested in FSHD1 primary cells (MB010, MB015, MB016, MB018 and MB020). In the complex, the anti-TfR1 Fab is covalently linked to the 5′ end of the sense strand of each siRNA via a linker, and the corresponding antisense strand is annealed to the sense strand. The anti-TfR1 Fab-siRNA complex comprises a structure of formula (Id):

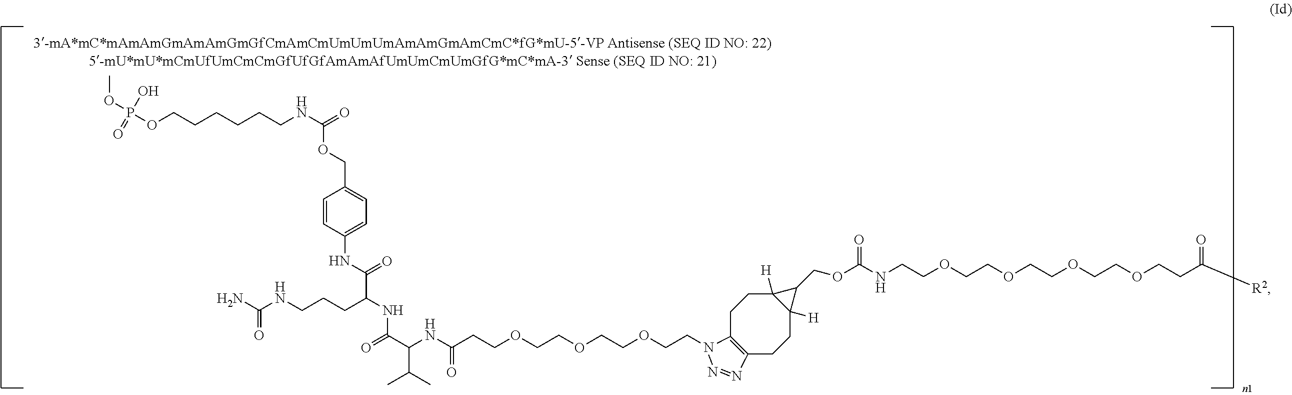

or a pharmaceutically acceptable salt thereof,
wherein: mA, mC, mG, and mU are 2′-O-methyl adenosine, 2′-O-methyl cytidine, 2′-O-methyl guanosine, and 2′-O-methyl uridine, respectively; fA, fC, fG, and fU are 2′-fluoro adenosine, 2′-fluoro cytidine, 2′-fluoro guanosine, and 2′-fluoro uridine, respectively; * between two nucleosides represents a phosphorothioate linkage; the absence of “*” between two nucleosides represents a phosphodiester internucleoside linkage; and “VP” represents 5′-(E)-Vinylphosphonate; wherein the oligonucleotide is an RNAi oligonucleotide (e.g., siRNA) comprising an antisense strand comprising a nucleobase sequence of UGCCAGAAUUUCACGGAAGAACA (SEQ ID NO: 22) and a sense strand comprising a nucleobase sequence of UUCUUCCGUGAAAUUCUGGCA (SEQ ID NO: 21); wherein R² comprises an antibody comprising a sequence as set forth in Table 2, and wherein in each complex, n1 is 1.

The primary cells were treated with the anti-TfR1 Fab-siRNA complex at a concentration equivalent of 100 nM of siRNA for 7 days. cDNA was obtained from cells with the TaqMan Fast Advanced Cells-to-Ct Kit (Thermo Fisher Scientific), and levels of three DUX4 transcriptome markers MBD3L2 (Hs00544743_m1), TRIM43 (Hs00299174_m1), ZSCAN4 (Hs00537549_m1), and RPL13A (Hs04194366_g1) were analyzed via qPCR with specific TaqMan assays (Thermo Fisher Scientific). Two-step amplification reactions and fluorescence measurements for determination of cycle threshold (Ct) were conducted on a QuantStudio 7 instrument (Thermo Fisher Scientific).

Results (see Table 3) show that the anti-TfR1 Fab-siRNA complex achieved significant reduction of the tested DUX4 transcriptome markers in FSHD patient-derived primary cells.

TABLE 3

DUX4 transcriptomics
(% control)

| FSHD cells | Average | SD |
|---|---|---|
| MB010 | 60.3 | 20.7 |
| MB015 | 33.7 | 12.0 |
| MB016 | 37.6 | 10.9 |
| MB018 | 34.5 | 13.4 |
| MB020 | 2.7 | 1.2 |

### Example 3: Activities of Anti-TfR1 Fab-siRNA Complexes in Mouse Muscle Tissues

Activities of the complexes described in Example 1, containing an anti-TfR1 antibody provided in Table 2 covalently linked via a linker to a DUX4-targeting siRNA, were tested in eight- to nine-week-old hTfR1/FLExDUX4/ACTA mice. hTfR1/FLExDUX4/ACTA mice are transgenic mice that carry a transgene encoding human hTfR1 protein, a Cre recombinase-activatable DUX4 expression vector, and a muscle-specific tamoxifen-inducible Cre recombinase expression vector. This system allows low level, spontaneous DUX4 activation in skeletal muscle in the absence of tamoxifen, or acute induction of DUX4 expression following tamoxifen administration. DUX4 activation in this mouse model causes expression of the DUX4 transcriptome (D4T). D4T activation therefore provides an indirect measurement of DUX4 levels, which is itself extremely difficult to detect.

Two versions of the anti-TfR1 Fab-siRNA complex were tested, one in which the anti-TfR1 Fab was covalently linked to the 3′ end of the sense strand of the siRNA and one in which the anti-TfR1 Fab was covalently linked to the 5′ end of the sense strand of each siRNA.

The hTfR1/FLExDUX4/ACTA mice were intravenously injected with a single dose of a vehicle control (phosphate-buffered saline) or an siRNA complex at a concentration equivalent to 10 mg/kg of siRNA. Each experimental condition was replicated in three to fifteen individual hTfR1/FLExDUX4/ACTA mice. Following twenty-eight days after injection, the mice were euthanized. Gastrocnemius and quadricep muscles were collected and snap frozen in RNAlater™ Stabilization Solution (Thermo Fisher Scientific). Total miRNA was isolated from gastrocnemius and quadricep muscles with the Maxwell® RSC miRNA tissue kit (Promega) and real-time quantitative RT-PCR was performed.

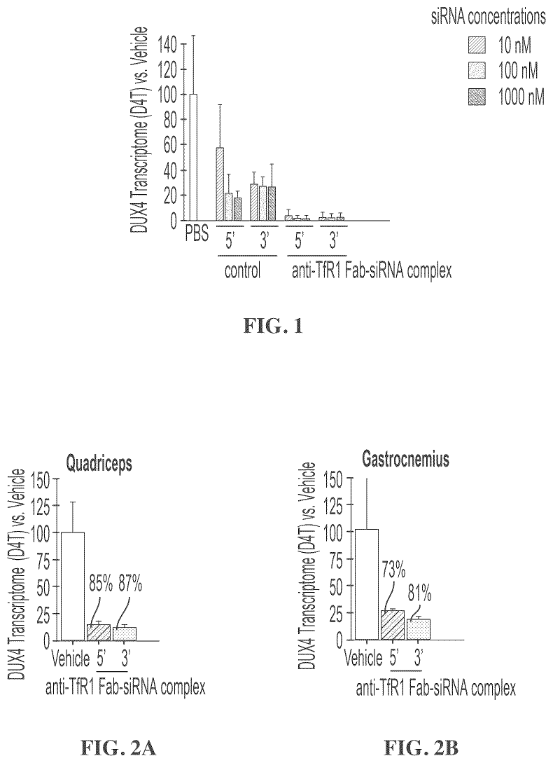

Three murine downstream DUX4 transcriptome markers Wfdc3 (Mm01243777_m1), Sord (Mm00455377_g1), Serpinb6c (Mm00655242_m1), and Rp137A (Mm00782745_s1) were analyzed via qPCR with specific TaqMan assays (Thermo Fisher Scientific). Two-step amplification reactions and fluorescence measurements for determination of cycle threshold (Ct) were conducted on a QuantStudio 7 instrument (Thermo Fisher Scientific). FSHD composite scores were calculated using the three DUX4 transcriptome markers (Wfdc3, Sord, Serpinb6c) where ΔCt=(Average Ct of 3 DUX4 markers)−(Average Ct of Rpl37A), ΔΔCt=ΔCt (Treated)−ΔCt (Vehicle), FSHD Composite=2−ΔΔCT*100(%) (see Table 4, FIGS. 2A and 2B).

Results show that the anti-TfR1 Fab-siRNA complexes achieved significant reduction of the tested DUX4 mouse transcriptome markers in the gastrocnemius and quadriceps of hTfR1/FLExDUX4/ACTA mice.

TABLE 4

| | DUX4 transcriptome knockdown (% knockdown vs | |
|---|---|---|
| | vehicle) | |
| antiTfR1-siRNA | Quadriceps | Gastrocnemius |
| siRNA - 5′ | 85 | 73 |
| siRNA - 3′ | 87 | 81 |

### Example 4: Stability and Binding Affinity of Anti-TfR1 Fab-siRNA Complexes in Exemplary Formulations

A formulation comprising complexes of formula (Id), or a pharmaceutically acceptable salt thereof, as described in Example 2, containing an anti-TfR1 antibody provided in Table 2 covalently linked to a DUX4-targeting siRNA was prepared. The anti-TfR1 Fab-siRNA complexes were formulated in a buffer at a Fab concentration of approximately 16 mg/mL. The buffer conditions were as follows: 25 mM tris(hydroxymethyl)aminomethane, 10% (w/v) sucrose, pH 7.5.

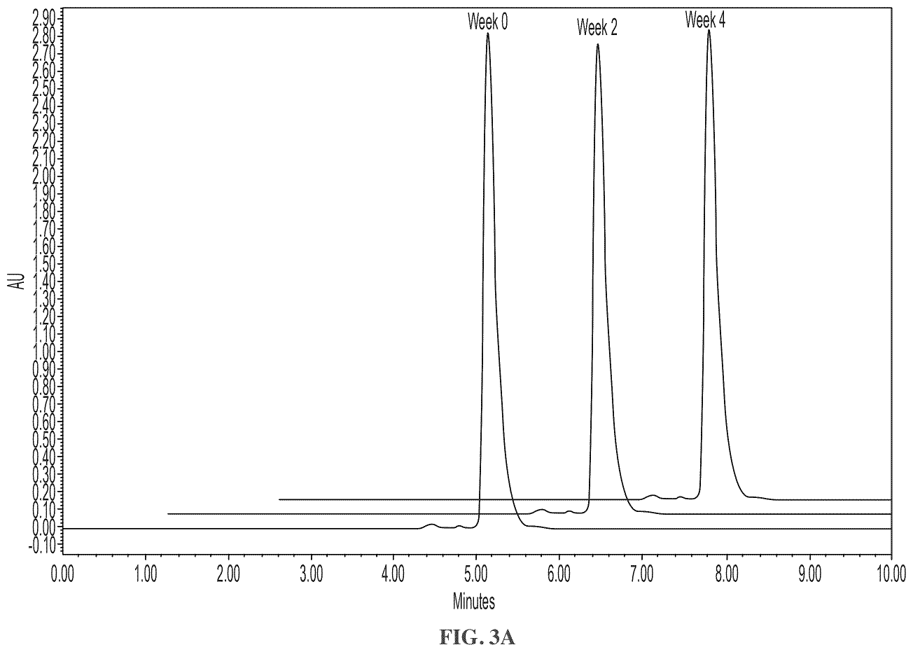

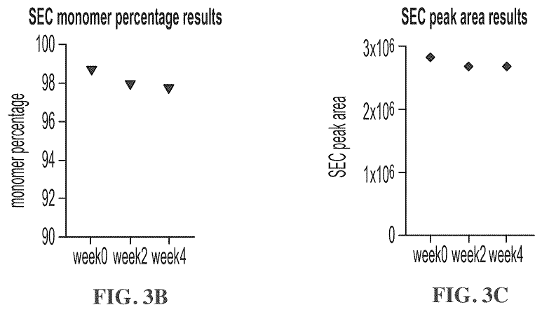

Heat stability of the complex formulated in the formulation buffer was tested by size exclusion chromatography (SEC). The formulation was incubated at room temperature for 4 weeks, and the samples were analyzed using analytical UPLC-SEC (Waters Xbridge Protein BEH SEC 2.5 um, 4.6×30 mm, 0.3 mL/min, in PBS) (see FIG. 3A). The monomer percentage of the DAR1 peak was measured to determine if the complex remained stable and intact (see FIG. 3B). The peak area was also measured to assess if the complex precipitated during the incubation period (see FIG. 3C).

The results in FIGS. 3A-3C showed that the complexes remained stable, with only slight changes observed in the monomer percentage and peak area. No changes occurred when the samples were kept at −20° C. and −80° C.

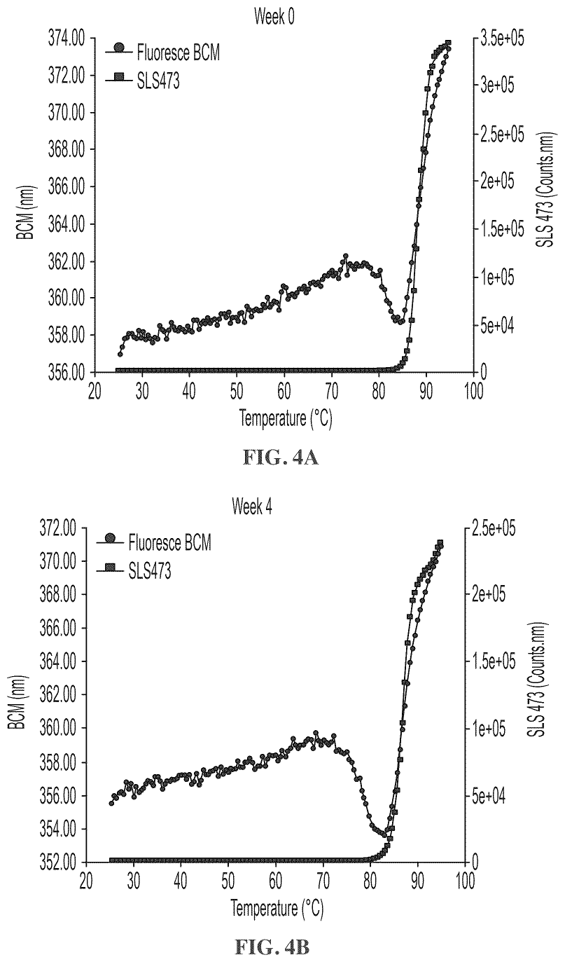

Additionally, heat stability of the complex formulated in the formulation buffer was measured by UNcle stability screening platform (Unchained Labs). UNcle combines full-spectrum fluorescence, Static Light Scattering (SLS) and Dynamic Light Scattering (DLS) measurement modes. Heat stability of the complex was measured by the UNcle Tₘ/T_{agg} method (9 ul of 10 mg/ml sample loaded in UNcle sample cartridge) (see FIGS. 4A-4B). FIGS. 4A and 4B show the results of BCM and SLS473 analyses for the complex at two different time points: week 0 and week 4 after incubation at room temperature. The BCM method analyzes the intrinsic protein fluorescence of the conjugate complex to characterize protein unfolding and determine the melting temperature (Tₘ) of the protein complex. The Tₘ values measured by BCM for the week 0 and week 4 samples are 86.8° C. and 86.5° C., respectively. SLS (s“atic light scatter”ng) uses “elastic scattering” to measure the scattering intensity of the protein complex, with intensity increasing as proteins aggregate. SLS473 provides the T_{agg} value, which represents the aggregation tendency of the protein complex. The T_{agg} values measured by SLS473 for the week 0 and week 4 samples are 82.4° C. and 82.8° C., respectively.

The results show that after incubation at room temperature for 4 weeks, the heat stability of the complex did not change.

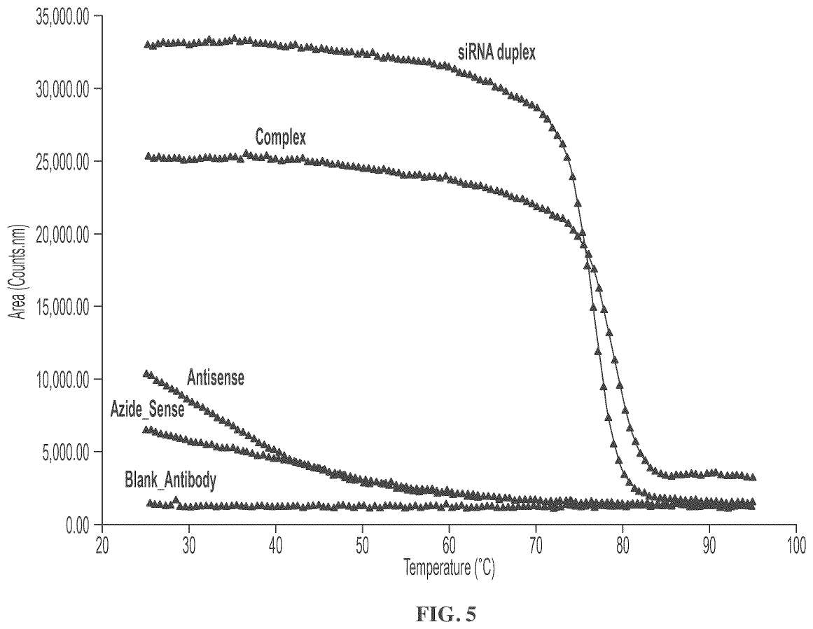

Differential scanning fluorimetry (DSF), an assay used to monitor the heat stability of RNA, was also run on UNcle. DSF measures the melting temperature (Tm) of siRNAs in their naked duplex form as well as when they are in the complex. The anti-TfR1 Fab-siRNA complex or the anti-TfR1 Fab were buffer exchanged into TE buffer and concentrated to 0.21 mM. They were then mixed with a diluted RiboGreen dye in TE buffer at a ratio of 1:1, with the RiboGreen dye being diluted 2000 times. DSF was run on UNcle using a pre-set program called “Tm by Sypro”. The fluorescence signals at 473 nm were applied to generate DSF profiles. The results are shown in FIG. 5 and demonstrate that both the naked siRNA duplex and the siRNA complex exhibit similar heat stability. The siRNA duplex was stable when the complex was kept at room temperature for four weeks. Melting temperatures measured at weeks 0, 2, and 4 were 78.3° C., 78.5° C., and 78.5° C., respectively.

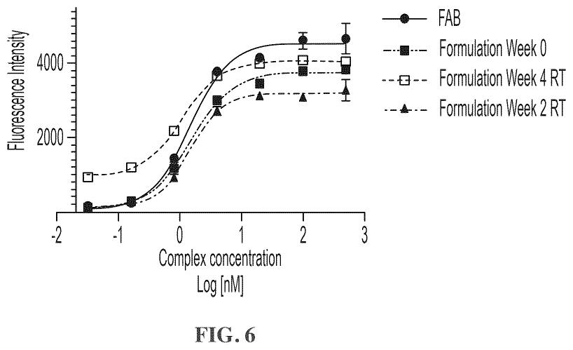

Binding affinity of the complex formulated in the formulation buffer was also tested by enzyme-linked immunosorbent assay (ELISA). The formulation was incubated at room temperature for four weeks. As shown in FIG. 6, the complex maintained its binding affinity to TfR1 after four weeks.

In addition to the above-discussed complexes in a formulation buffer comprising 25 mM tris(hydroxymethyl)aminomethane and 10% (w/v) sucrose, with a pH of 7.5, two alternative formulations were also tested. The buffer conditions in each of the alternative formulations were as follows:
Alternative Formulation a: 25 mM Tris, 3% (w/v) sucrose, pH 7.5.
Alternative Formulation b: 25 mM Tris, 6% (w/v) sucrose+50 mM NaCl, pH 7.5.

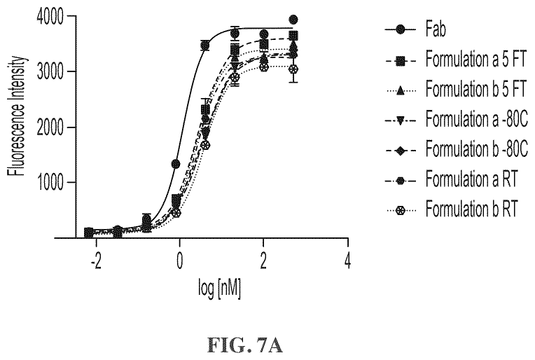

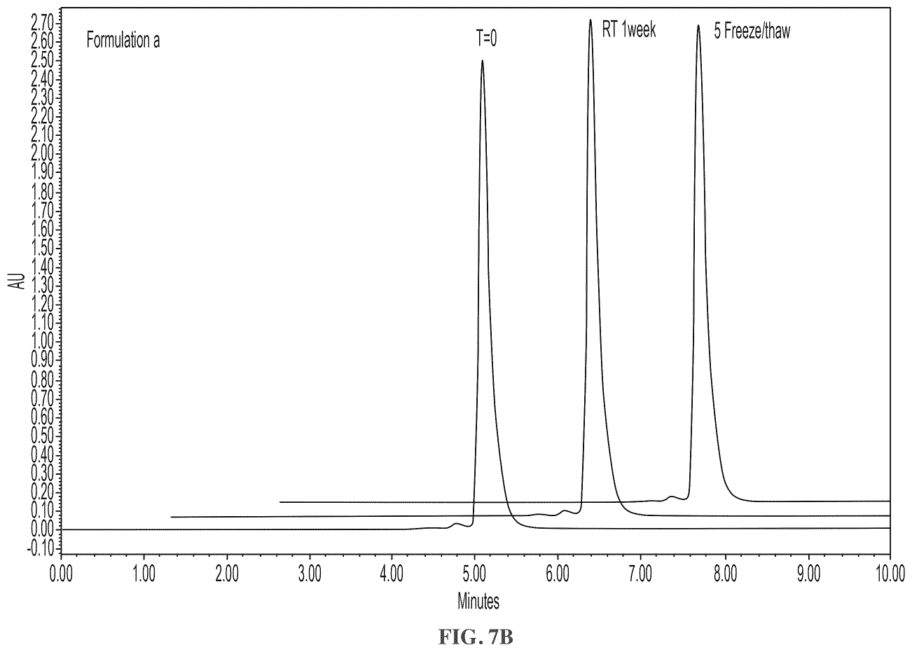

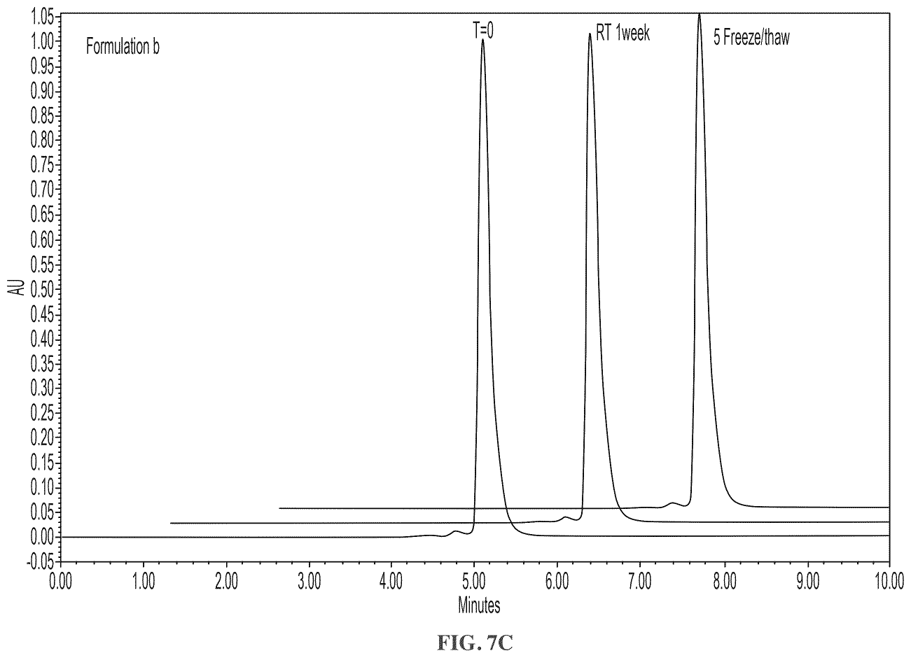

The formulated samples were stored at −80° C. and room temperature (RT) for one week, and no changes were observed when the samples were tested using SEC, ELISA, DLS, and DSF (see FIGS. 7A-7C).

Additionally, the anti-TfR1 Fab-siRNA complexes formulated in the three formulation buffers were subjected to five freeze/thaw cycles, and no changes in stability were found (FIG. 7C).

The DUX4-targeting siRNAs described herein were also tested for stability in human serum and human liver lysate and were confirmed to be stable in human serum and human liver lysate. Immunostimulatory properties of the DUX4-targeting siRNAs were also evaluated in human peripheral blood mononuclear cells (PBMCs) and did not show strong immunogenicity. Lastly, an off-target assessment in human primary hepatocytes was conducted and the DUX4-targeting siRNAs showed reduced off-target effects compared to other known DUX4-targeting siRNAs.

### Additional Embodiments

1. A composition comprising complexes that comprise an oligonucleotide covalently linked to an anti-transferrin receptor 1 (TfR1) antibody,
wherein the anti-TfR1 antibody comprises: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16,
wherein the oligonucleotide is an RNAi oligonucleotide.

2. A composition comprising complexes comprising a structure of formula (I): [R¹]ₙ₁—R², wherein each R¹ independently comprises a group of the formula (Ia):

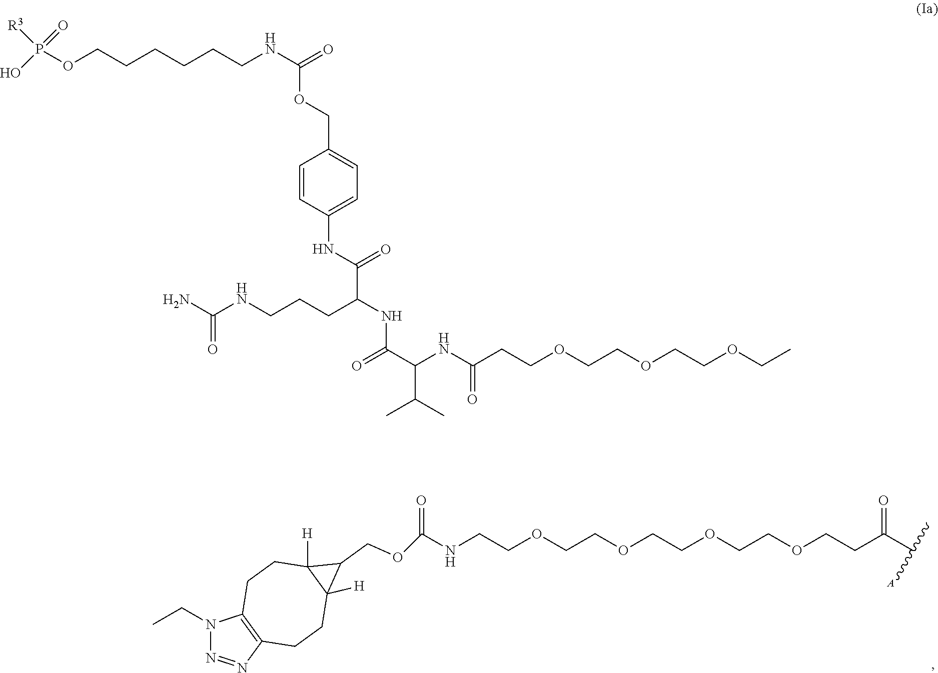

or a pharmaceutically acceptable salt thereof,
wherein
   R² comprises an antibody, and
   R³ comprises an RNAi oligonucleotide;
   wherein R¹ is covalently linked to R² at attachment point A; and
   wherein n1 is an integer representing the number of instances of R¹, wherein each instance of R¹ is covalently linked to a different amino acid residue of the antibody, optionally wherein each different amino acid residue is a lysine;
   optionally wherein the antibody is an anti-TfR1 antibody.

3. The composition of embodiment 2, wherein the antibody comprises:
a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16.

4. The composition of any one of embodiments 1 to 3, wherein the composition is a formulation comprising tris(hydroxymethyl)aminomethane and sucrose.

5. The composition of embodiment 4, wherein the formulation is in a lyophilized form.

6. The composition of embodiment 4, wherein the formulation is in a frozen solid form.

7. The composition of embodiment 4, wherein the formulation is in an aqueous solution.

8. The composition of embodiment 7, wherein the tris(hydroxymethyl)aminomethane is present in the aqueous solution at a concentration in the range of 5 mM to 50 mM.

9. The composition of embodiment 7 or 8, wherein the sucrose is present in the aqueous solution at a concentration in the range of 5% to 15% weight per volume (w/v %).

10. The composition of any one of embodiments 7 to 9, wherein the aqueous solution has a pH in the range of 6.5 to 8.5.

11. The composition of any one of embodiments 7 to 10, wherein the tris(hydroxymethyl)aminomethane is present in the aqueous solution at a concentration of 25 mM and/or the sucrose is present in the aqueous solution at a concentration of 10 w/v % and/or the aqueous solution is at a pH of 7.5.

12. The composition of any one of embodiments 1-11, wherein the antibody is a Fab fragment, a full-length IgG, a Fab′ fragment, a F(ab′)₂ fragment, an scFv, or an Fv.

13. The composition of embodiment 12, wherein the antibody is a Fab fragment.

14. The composition of any one of embodiments 1-13, wherein the antibody comprises a heavy chain variable region (VH) comprising an amino acid sequence at least 85% identical to SEQ ID NO: 17; and/or wherein the antibody comprises a light chain variable region (VL) comprising an amino acid sequence at least 85% identical to SEQ ID NO: 18,
optionally wherein the antibody comprises a VH comprising the amino acid sequence of SEQ ID NO: 17 and/or a VL comprising the amino acid sequence of SEQ ID NO: 18.

15. The composition of embodiment 14, wherein the VH comprises an N-terminal pyroglutamate.

16. The composition of any one of embodiments 1-14, wherein the antibody comprises a heavy chain comprising an amino acid sequence at least 85% identical to SEQ ID NO: 19; and/or wherein the antibody comprises a light chain comprising an amino acid sequence at least 85% identical to SEQ ID NO: 20,
optionally wherein the antibody comprises a heavy chain comprising the amino acid sequence of SEQ ID NO: 19 and/or a light chain comprising the amino acid sequence of SEQ ID NO: 20.

17. The composition of any one of embodiments 1-16, wherein the RNAi oligonucleotide comprises an antisense strand of 18-25 nucleosides in length.

18. The composition of any one of embodiments 1-17, wherein the RNAi oligonucleotide comprises an antisense strand comprising a region of complementarity of at least 16 consecutive nucleosides in length to a nucleobase sequence as set forth in SEQ ID NO: 24, optionally wherein the region of complementarity is 22 consecutive nucleosides in length.

19. The composition of any one of embodiments 1-18, wherein the RNAi oligonucleotide comprises an antisense strand comprising at least 15 consecutive nucleosides of a nucleobase sequence as set forth in SEQ ID NO: 22, optionally wherein the antisense strand comprises the nucleobase sequence of SEQ ID NO: 22.

20. The composition of any one of embodiments 1-19, wherein the RNAi oligonucleotide further comprises a sense strand comprising at least 15 consecutive nucleosides complementary to the antisense strand.

21. The composition of any one of embodiments 1-20, wherein the sense strand comprises at least 15 consecutive nucleosides of a nucleobase sequence as set forth in SEQ ID NO: 21, optionally wherein the sense strand comprises the nucleobase sequence of SEQ ID NO: 21.

22. The composition of any one of embodiments 1-21, wherein the RNAi oligonucleotide comprises one or more modified nucleosides.

23. The composition of embodiment 22, wherein the one or more modified nucleosides are 2′ modified nucleosides, optionally, wherein each 2′ modified nucleoside is 2′-O-methyl (2′-O-Me) or 2′-fluoro (2′-F).

24. The composition of any one of embodiments 1-23, wherein the antisense strand further comprises a 5′-(E)-vinylphosphonate.

25. The composition of any one of embodiments 1-24, wherein the RNAi oligonucleotide comprises one or more phosphorothioate internucleoside linkages.

26. The composition of any one of embodiments 2-25, wherein each R¹ comprises a group of the formula (Ib):

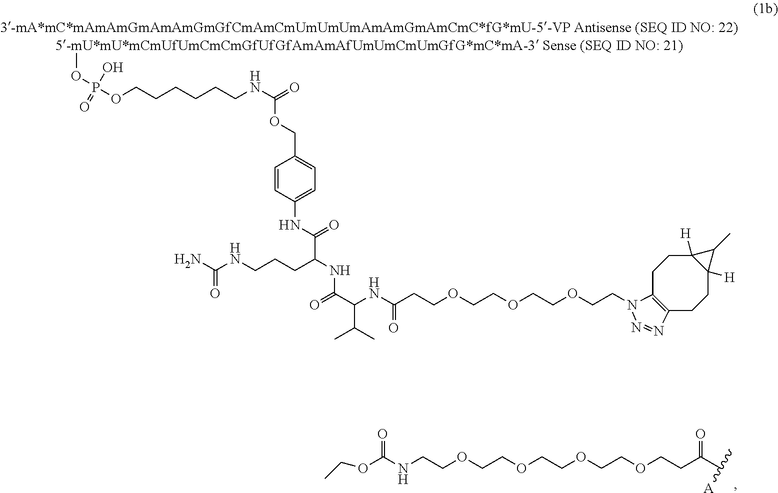

or a pharmaceutically acceptable salt thereof,
wherein: mA, mC, mG, and mU are 2′-O-methyl adenosine, 2′-O-methyl cytidine, 2′-O-methyl guanosine, and 2′-O-methyl uridine, respectively;
fA, fC, fG, and fU are 2′-fluoro adenosine, 2′-fluoro cytidine, 2′-fluoro guanosine, and 2′-fluoro uridine, respectively; * between two nucleosides represents a phosphorothioate linkage; the absence of “*” between two nucleosides represents a phosphodiester internucleoside linkage; and “VP” represents 5′-(E)-Vinylphosphonate, such that the RNAi oligonucleotide comprises an antisense strand comprising a nucleobase sequence of UGCCAGAAUUUCACGGAAGAACA (SEQ ID NO: 22) and a sense strand comprising a nucleobase sequence of UUCUUCCGUGAAAUUCUGGCA (SEQ ID NO: 21).

27. The composition of any one of embodiments 2-25, wherein each R¹ comprises a group of the formula (Ic), in which the antisense strand and the sense strand form a double stranded oligonucleotide:

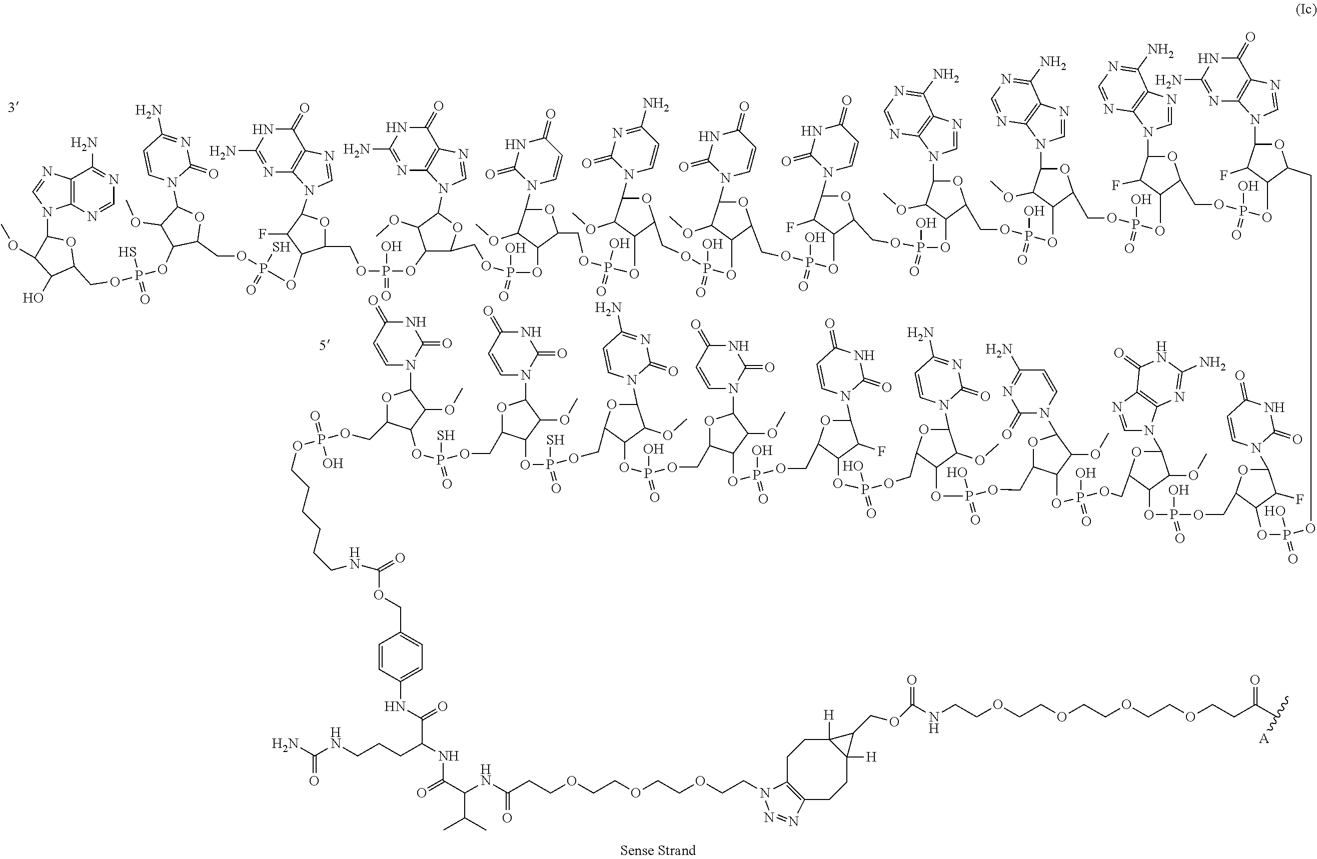

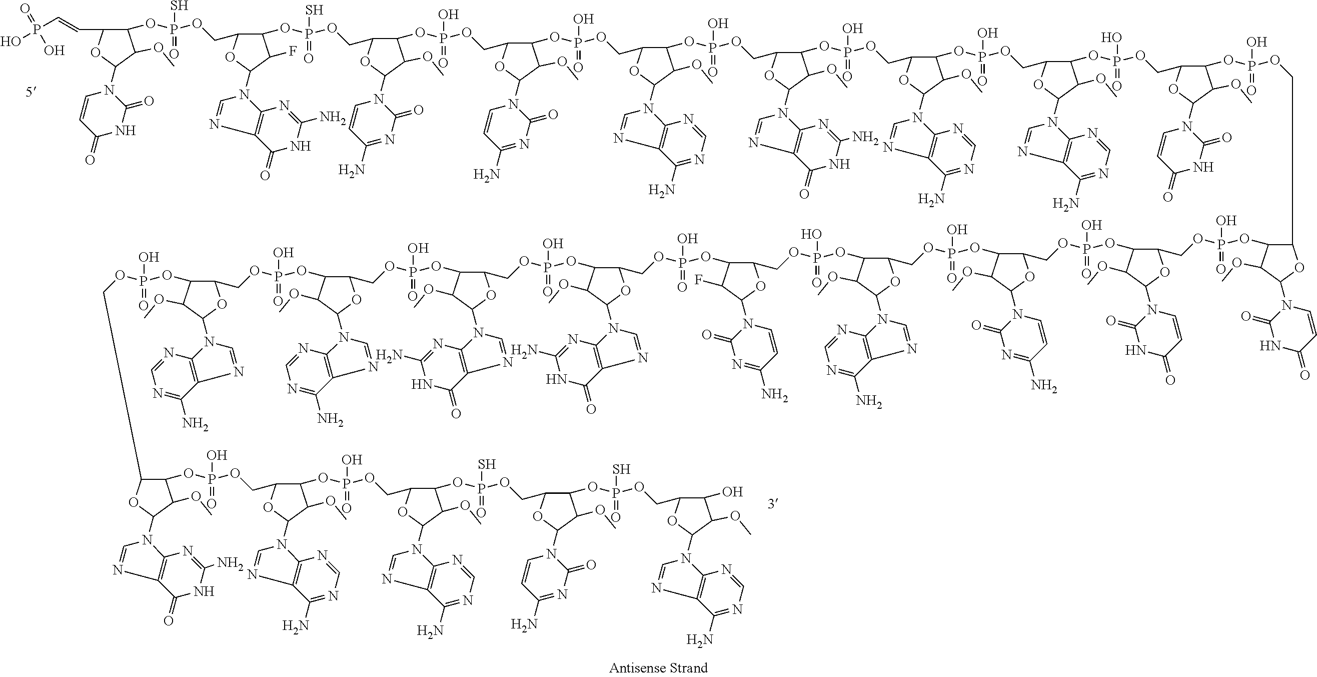

or a pharmaceutically acceptable salt thereof.

28. The composition of any one of embodiments 1-27, wherein the complexes are present in the composition at a concentration in the range of 10 mg/mL to 200 mg/mL.

29. The composition of any one of embodiments 1-28, further comprising one or more antibodies that are not covalently linked to an oligonucleotide.

30. The composition of embodiment 29, wherein the average value of n1 of complexes in the composition is in the range of 0.5 to 5, optionally wherein the average value of n1 of complexes in the composition is 1.

31. A method of reducing DUX4 expression in a subject, the method comprising administering to the subject an effective amount of the composition of any one of embodiments 1-30.

32. A method of treating facioscapulohumeral muscular dystrophy (FSHD) in a subject, the method comprising administering to the subject an effective amount of the composition ofany one of embodiments 1-30.

33. The method of embodiment 31 or embodiment 32, wherein the subject has aberrant production of DUX4 protein.

34. The method of any one of embodiments 31-33, wherein the complexes reduce DUX4 expression in the subject.

35. The method of embodiment 34, wherein reducing DUX4 expression comprises reducing DUX4 protein and/or mRNA levels.

36. The method of any one of embodiments 31 to 35, wherein the antibody comprises a VH that comprises an N-terminal pyroglutamate.

37. A complex comprising a structure of formula (I): [R¹]ₙ₁—R², wherein each R¹ comprises a group of the formula (Ia):

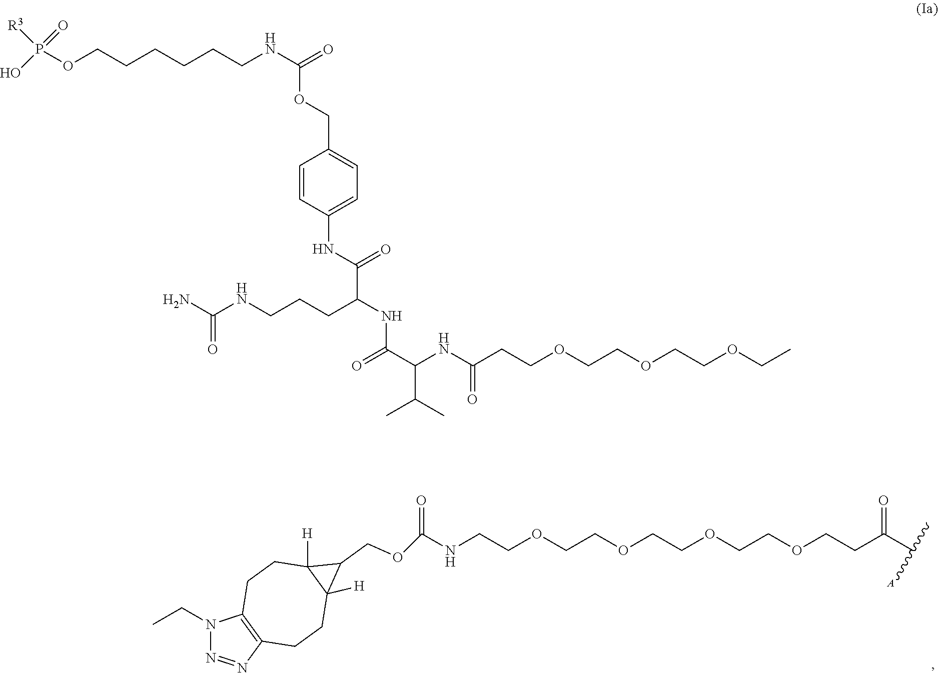

or a pharmaceutically acceptable salt thereof,
wherein R³ comprises an RNAi oligonucleotide comprising an antisense strand comprising a nucleobase sequence of SEQ ID NO: 22 and a structure (5′→3′) of VP-mU*fG*mCmCmAmGmAmAmUmUmUmCmAfCmGmGmAmAmGmAmA*mC*mA, and
   a sense strand comprising a nucleobase sequence of SEQ ID NO: 21 and a structure (5′→3′) of mU*mU*mCmUfUmCmCmGfUfGfAmAmAfUmUmCmUmGfG*mC*mA, wherein: mA, mC, mG, and mU are 2′-O-methyl adenosine, 2′-O-methyl cytidine, 2′-O-methyl guanosine, and 2′-O-methyl uridine, respectively; fA, fC, fG, and fU are 2′-fluoro adenosine, 2′-fluoro cytidine, 2′-fluoro guanosine, and 2′-fluoro uridine, respectively; * between two nucleosides represents a phosphorothioate linkage; the absence of “*” between two nucleosides represents a phosphodiester internucleoside linkage; and “VP” represents 5′-(E)-Vinylphosphonate;
   wherein R² comprises a Fab, and wherein the Fab comprises a CDR-H1, a CDR-H2, a CDR-H3, a CDR-L1, a CDR-L2, and a CDR-L3 selected from Table 2,
   optionally wherein the Fab comprises a VH comprising the amino acid sequence of SEQ ID NO: 17 and a VL comprising the amino acid sequence of SEQ ID NO: 18,
   further optionally wherein the Fab comprises a VH comprising an N-terminal pyroglutamate,
   further optionally wherein the Fab comprises a heavy chain comprising the amino acid sequence of SEQ ID NO: 19 and a light chain comprising the amino acid sequence of SEQ ID NO: 20;
   wherein R¹ is covalently linked to R² at attachment point A; and wherein n1 is an integer representing the number of instances of R¹, wherein each instance of R¹ is covalently linked to a different amino acid residue of the Fab, optionally wherein each different amino acid residue is a lysine.

38. A complex comprising a structure of formula (I): [R¹]ₙ₁—R², wherein
each R¹ comprises a group of the formula (Ib):

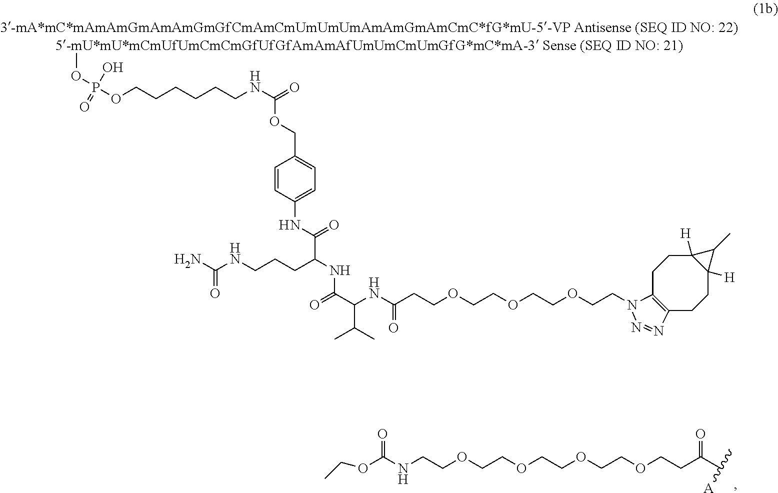

or a pharmaceutically acceptable salt thereof,
wherein: mA, mC, mG, and mU are 2′-O-methyl adenosine, 2′-O-methyl cytidine, 2′-O-methyl guanosine, and 2′-O-methyl uridine, respectively; fA, fC, fG, and fU are 2′-fluoro adenosine, 2′-fluoro cytidine, 2′-fluoro guanosine, and 2′-fluoro uridine, respectively; * between two nucleosides represents a phosphorothioate linkage; the absence of “*” between two nucleosides represents a phosphodiester internucleoside linkage; and “VP” represents 5′-(E)-Vinylphosphonate, such that the oligonucleotide is an RNAi oligonucleotide comprising an antisense strand comprising a nucleobase sequence of UGCCAGAAUUUCACGGAAGAACA (SEQ ID NO: 22) and a sense strand comprising a nucleobase sequence of UUCUUCCGUGAAAUUCUGGCA (SEQ ID NO: 21);
   wherein R² comprises a Fab, and wherein the Fab comprises a CDR-H1, a CDR-H2, a CDR-H3, a CDR-L1, a CDR-L2, and a CDR-L3 selected from Table 2,
   optionally wherein the Fab comprises a VH comprising the amino acid sequence of SEQ ID NO: 17 and a VL comprising the amino acid sequence of SEQ ID NO: 18,
   further optionally wherein the Fab comprises a VH comprising an N-terminal pyroglutamate,
   further optionally wherein the Fab comprises a heavy chain comprising the amino acid sequence of SEQ ID NO: 19 and a light chain comprising the amino acid sequence of SEQ ID NO: 20;
   wherein R¹ is covalently linked to R² at attachment point A; and wherein n1 is an integer representing the number of instances of R¹, wherein each instance of R¹ is covalently linked to a different amino acid residue of the Fab, optionally wherein each different amino acid residue is a lysine.

39. A complex comprising a structure of formula (I): [R¹]ₙ₁—R², wherein
R¹ comprises a group of the formula (Ic), in which the antisense strand and the sense strand form a double stranded oligonucleotide:

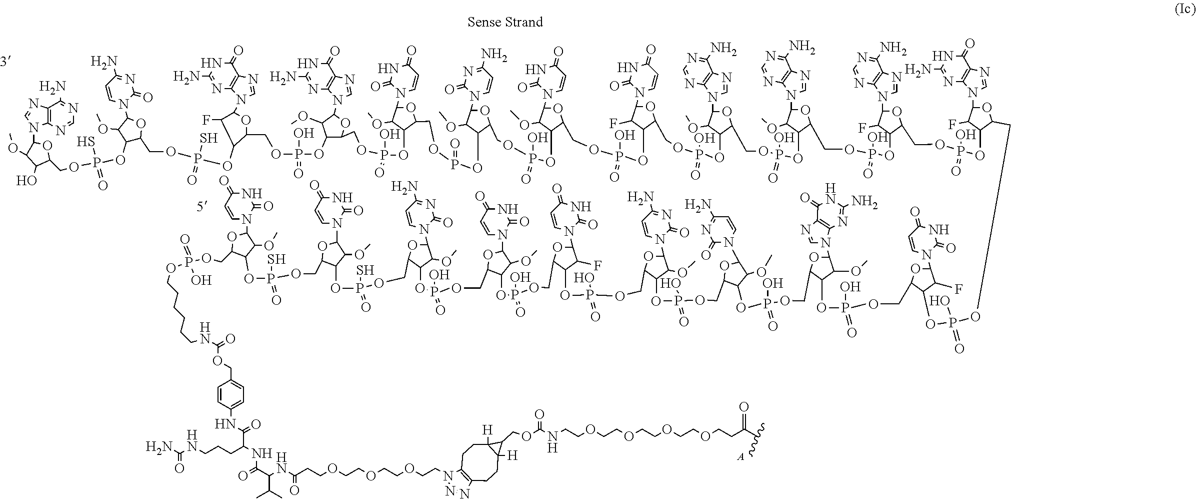

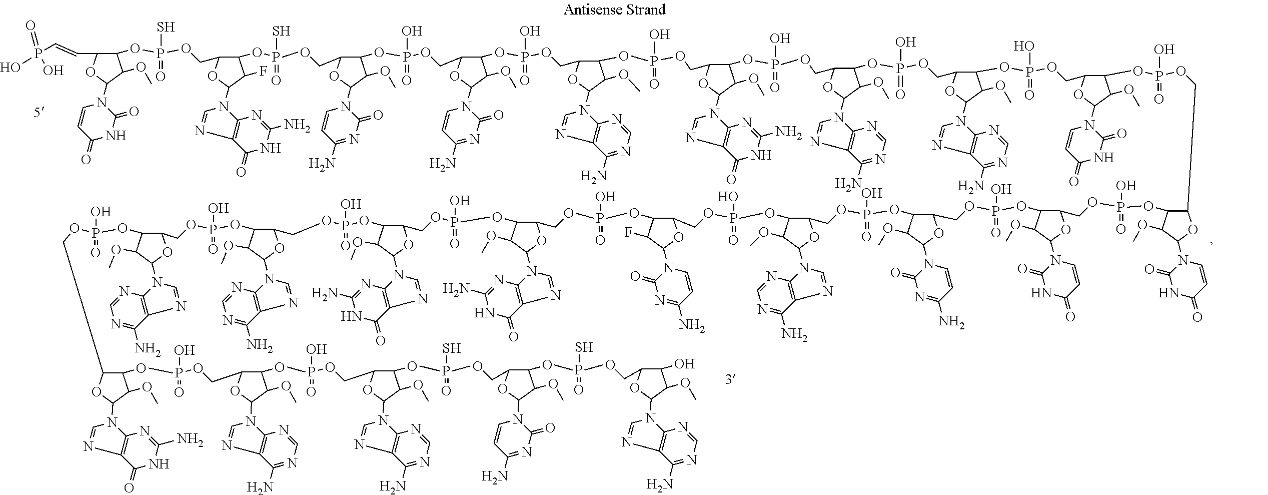

or a pharmaceutically acceptable salt thereof,
   wherein R² comprises a Fab comprising a CDR-H1, a CDR-H2, a CDR-H3, a CDR-L1, a CDR-L2, and a CDR-L3 selected from Table 2,
   optionally wherein the Fab comprises a VH comprising the amino acid sequence of SEQ ID NO: 17 and a VL comprising the amino acid sequence of SEQ ID NO: 18,
   further optionally wherein the Fab comprises a VH comprising an N-terminal pyroglutamate,
   further optionally wherein the Fab comprises a heavy chain comprising the amino acid sequence of SEQ ID NO: 19 and a light chain comprising the amino acid sequence of SEQ ID NO: 20; wherein R¹ is covalently linked to R² at attachment point A;
   wherein n1 is an integer representing the number of instances of R¹, wherein each instance of R¹ is covalently linked to a different amino acid residue of the Fab, optionally wherein each different amino acid residue is a lysine.

40. A complex comprising a structure of the formula (Id):

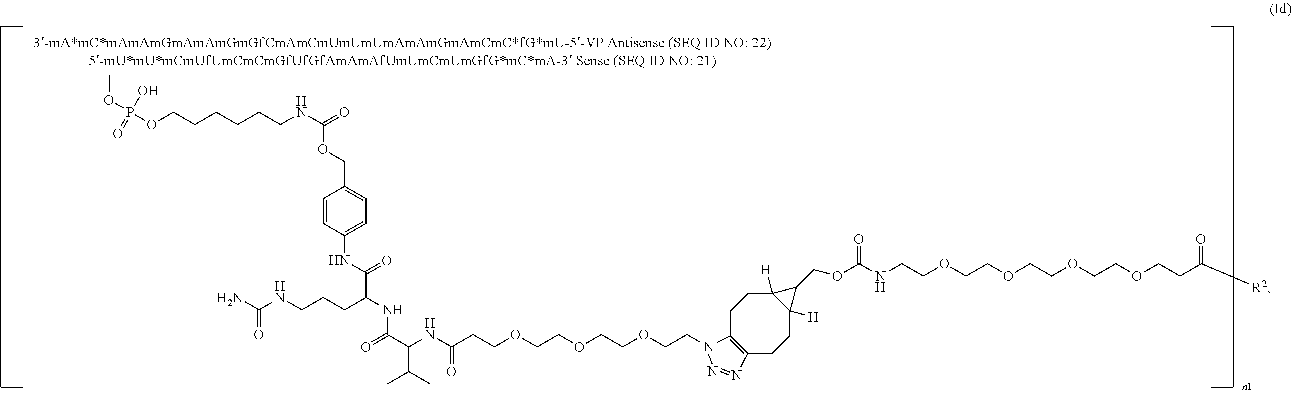

or a pharmaceutically acceptable salt thereof,
wherein: mA, mC, mG, and mU are 2′-O-methyl adenosine, 2′-O-methyl cytidine, 2′-O-methyl guanosine, and 2′-O-methyl uridine, respectively;
fA, fC, fG, and fU are 2′-fluoro adenosine, 2′-fluoro cytidine, 2′-fluoro guanosine, and 2′-fluoro uridine, respectively; * between two nucleosides represents a phosphorothioate linkage; the absence of “*” between two nucleosides represents a phosphodiester internucleoside linkage; and “VP” represents 5′-(E)-Vinylphosphonate, such that the oligonucleotide is an RNAi oligonucleotide comprising an antisense strand comprising a nucleobase sequence of UGCCAGAAUUUCACGGAAGAACA (SEQ ID NO: 22) and a sense strand comprising a nucleobase sequence of UUCUUCCGUGAAAUUCUGGCA (SEQ ID NO: 21;
wherein R² comprises a Fab, and wherein the Fab comprises a CDR-H1, a CDR-H2, a CDR-H3, a CDR-L1, a CDR-L2, and a CDR-L3 selected from Table 2,
optionally wherein the Fab comprises a VH comprising the amino acid sequence of SEQ ID NO: 17 and a VL comprising the amino acid sequence of SEQ ID NO: 18,
further optionally wherein the Fab comprises a VH comprising an N-terminal pyroglutamate,
further optionally wherein the Fab comprises a heavy chain comprising the amino acid sequence of SEQ ID NO: 19 and a light chain comprising the amino acid sequence of SEQ ID NO: 20;
wherein n1 is an integer representing the number of instances of the group enclosed by square brackets, wherein each instance of the group enclosed by square brackets is covalently linked to a different amino acid residue of the Fab, optionally wherein each different amino acid residue is a lysine.

41. A formulation comprising a plurality of complexes of any one of embodiments 37-40, and tris(hydroxymethyl)aminomethane at a concentration of 5 to 50 mM, and sucrose at a concentration of 2 w/v % to 15 w/v %, wherein the formulation is an aqueous solution and is at a pH of 6.5 to 8.5, optionally wherein the plurality of complexes are at a concentration of 10 mg/mL to 200 mg/mL.

42. A formulation comprising a plurality of complexes of any one of embodiments 37-40, and tris(hydroxymethyl)aminomethane at a concentration of 5 to 50 mM, and sucrose at a concentration of 3 w/v % to 10 w/v %, wherein the formulation is an aqueous solution and is at a pH of 6.5 to 8.5, optionally wherein the plurality of complexes are at a concentration of 10 mg/mL to 200 mg/mL.

43. A formulation comprising a plurality of complexes of any one of embodiments 37-40, and tris(hydroxymethyl)aminomethane at a concentration of 25 mM, and sucrose at a concentration of 10 w/v %, wherein the formulation is an aqueous solution and is at a pH of 7.5, optionally wherein the plurality of complexes are at a concentration of 10 mg/mL to 200 mg/mL.

44. The formulation of any one of embodiments 41-43, further comprising one or more antibodies that are not covalently linked to an oligonucleotide.

45. The formulation of embodiment 44, wherein the average value of n1 of complexes in the formulation is in the range of 0.5 to 5, optionally wherein the average value of n1 of complexes in the formulation is 1.

46. A lyophilized form of the formulation of any one of embodiments 41-45.

47. A product produced by a process comprising lyophilizing the formulation of any one of embodiments 41-45.

48. A frozen form of the formulation of any one of embodiments 41-45.

49. A product produced by a process comprising freezing the formulation of any one of embodiments 41-45.

50. A lyophilized cake comprising a plurality of complexes of any one of embodiments 37-40, tris(hydroxymethyl)aminomethane, and sucrose.

### EQUIVALENTS AND TERMINOLOGY

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure. Thus, it should be understood that although the present disclosure has been specifically disclosed by preferred embodiments, optional features, modification and variation of the concepts herein disclosed may be resorted to by those skilled in the art, and that such modifications and variations are considered to be within the scope of this disclosure.

In addition, where features or aspects of the disclosure are described in terms of Markush groups or other grouping of alternatives, those skilled in the art will recognize that the disclosure is also thereby described in terms of any individual member or subgroup of members of the Markush group or other group.

It should be appreciated that, in some embodiments, sequences presented in the sequence listing may be referred to in describing the structure of an oligonucleotide or other nucleic acid. In such embodiments, the actual oligonucleotide or other nucleic acid may have one or more alternative nucleosides (e.g., an RNA counterpart of a DNA nucleoside or a DNA counterpart of an RNA nucleoside) and/or (e.g., and) one or more modified nucleosides and/or (e.g., and) one or more modified internucleoside linkages and/or (e.g., and) one or more other modification compared with the specified sequence while retaining essentially same or similar complementary properties as the specified sequence.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. Unless otherwise indicated, ranges of values herein are inclusive of their endpoints (e.g., a range of X to Y is inclusive of the values X and Y). It should be understood that recitations herein of a value from X to Y indicates that the specified value falls in the range of X to Y. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein. Variations of those embodiments may become apparent to those of ordinary skill in the art upon reading the foregoing description.

The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. Those skilled in the art will recognize, or be able to ascertain using no more than routine experimentation, many equivalents to the specific embodiments of the invention described herein. Such equivalents are intended to be encompassed by the following claims.

1-30. (canceled)

31. A complex comprising a structure of formula (I): [R¹]ₙ₁—R², wherein each R¹ comprises a group of the formula (Ic), in which the antisense strand and the sense strand form a double stranded oligonucleotide: 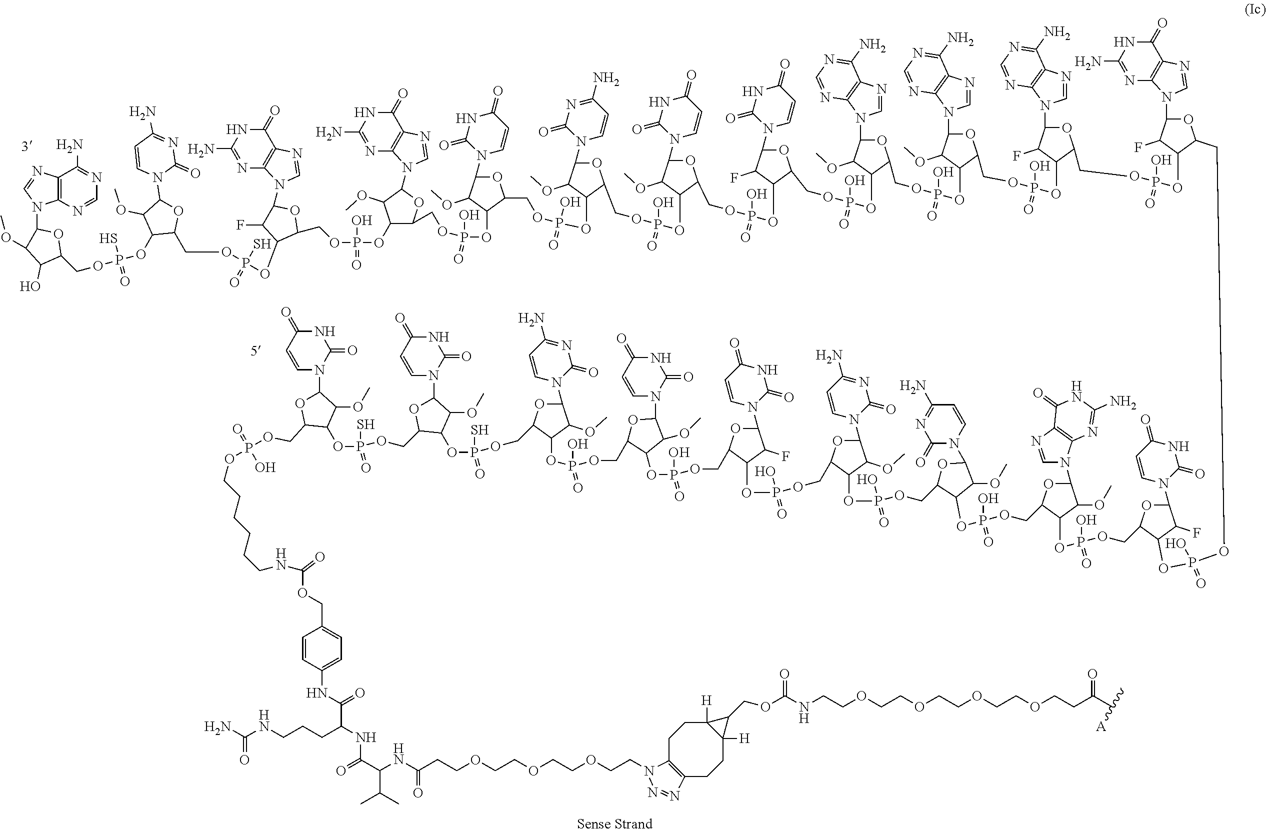 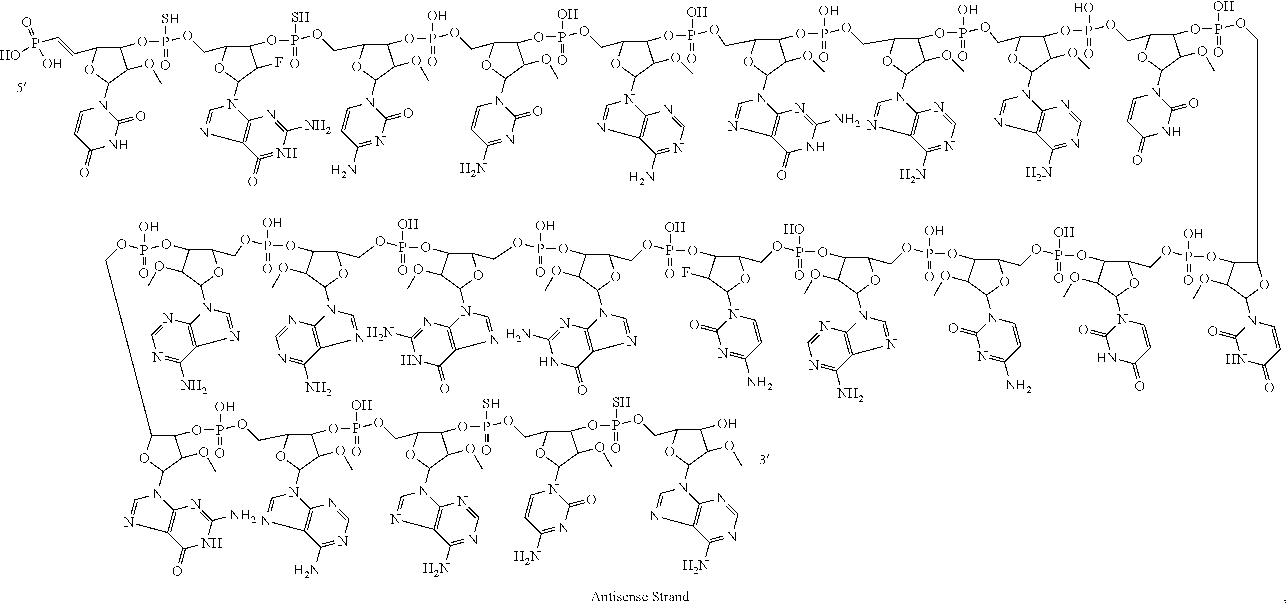
or a pharmaceutically acceptable salt thereof,
wherein R² comprises a Fab comprising a heavy chain comprising the amino acid sequence of SEQ ID NO: 19 and a light chain comprising the amino acid sequence of SEQ ID NO: 20;
wherein R¹ is covalently linked at attachment point A to R²; wherein n1 is an integer representing the number of instances of R¹, and wherein each instance of R¹ is covalently linked to a different amino acid residue of the Fab.

32. The complex of claim 31, wherein each of the different amino acid residue is a lysine.

33. The complex of claim 31, wherein the heavy chain of the Fab comprises an N-terminal pyroglutamate.

34. A method of reducing DUX4 expression in a subject, the method comprising administering to the subject a composition comprising the complex of claim 31.

35. The method of claim 34, wherein reducing DUX4 expression comprises reducing DUX4 protein and/or DUX4 mRNA levels.

36. A method of treating facioscapulohumeral muscular dystrophy (FSHD) in a subject, the method comprising administering to the subject a composition comprising the complex of claim 31.

37. The method of claim 36, wherein the subject has aberrant production of DUX4 protein.

38. A complex comprising a structure of formula (I): [R¹]ₙ₁—R², wherein each R¹ comprises a group of the formula (Ia): 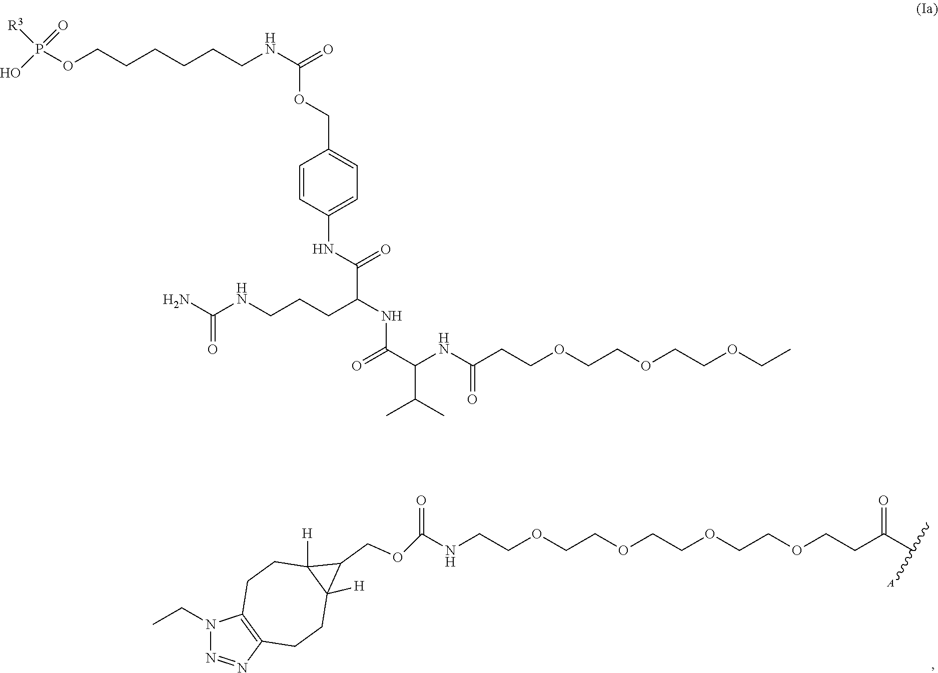
or a pharmaceutically acceptable salt thereof,
wherein R³ comprises an RNAi oligonucleotide comprising an antisense strand comprising a nucleobase sequence of SEQ ID NO: 22 and a structure (5′→3′) of VP-mU*fG*mCmCmAmGmAmAmUmUmUmCmAfCmGmGmAmAmGmAmA*mC*mA, and
a sense strand comprising a nucleobase sequence of SEQ ID NO: 21 and a structure (5′→3′) of mU*mU*mCmUfUmCmCmGfUfGfAmAmAfUmUmCmUmGfG*mC*mA, wherein: mA, mC, mG, and mU are 2′-O-methyl adenosine, 2′-O-methyl cytidine, 2′-O-methyl guanosine, and 2′-O-methyl uridine, respectively; fA, fC, fG, and fU are 2′-fluoro adenosine, 2′-fluoro cytidine, 2′-fluoro guanosine, and 2′-fluoro uridine, respectively; “*” between two nucleosides represents a phosphorothioate linkage; the absence of “*” between two nucleosides represents a phosphodiester internucleoside linkage; and “VP” represents 5′-(E)-Vinylphosphonate;
wherein R² comprises a Fab comprising a heavy chain comprising the amino acid sequence of SEQ ID NO: 19 and a light chain comprising the amino acid sequence of SEQ ID NO: 20;
wherein R¹ is covalently linked at attachment point A to R²; wherein n1 is an integer representing the number of instances of R¹, and wherein each instance of R¹ is covalently linked to a different amino acid residue of the Fab.

39. The complex of claim 38, wherein each of the different amino acid residue is a lysine.

40. The complex of claim 38, wherein the heavy chain of the Fab comprises an N-terminal pyroglutamate.

41. A method of reducing DUX4 expression in a subject, the method comprising administering to the subject a composition comprising the complex of claim 38.

42. The method of claim 41, wherein reducing DUX4 expression comprises reducing DUX4 protein and/or DUX4 mRNA levels.

43. A method of treating facioscapulohumeral muscular dystrophy (FSHD) in a subject, the method comprising administering to the subject a composition comprising the complex of claim 38.

44. The method of claim 43, wherein the subject has aberrant production of DUX4 protein.

45. A complex comprising a structure of formula (I): [R¹]ₙ₁—R², wherein each R¹ comprises a group of the formula (Ib): 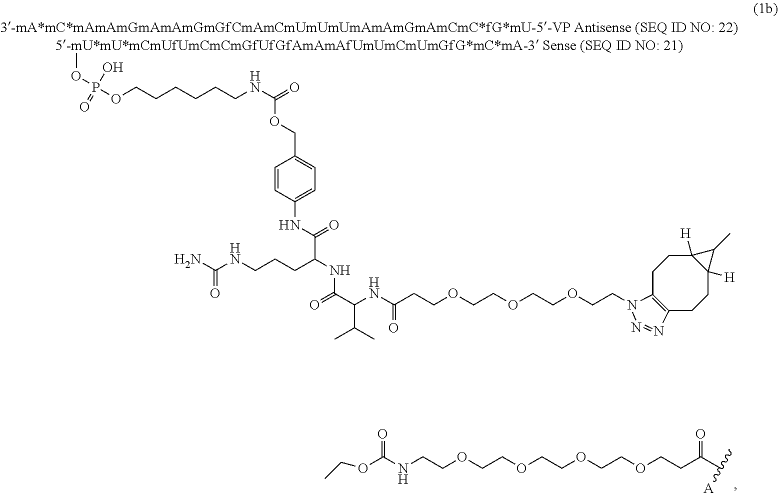
or a pharmaceutically acceptable salt thereof,
wherein: mA, mC, mG, and mU are 2′-O-methyl adenosine, 2′-O-methyl cytidine, 2′-O-methyl guanosine, and 2′-O-methyl uridine, respectively; fA, fC, fG, and fU are 2′-fluoro adenosine, 2′-fluoro cytidine, 2′-fluoro guanosine, and 2′-fluoro uridine, respectively; “*” between two nucleosides represents a phosphorothioate linkage; the absence of “*” between two nucleosides represents a phosphodiester internucleoside linkage; and “VP” represents 5′-(E)-Vinylphosphonate;
wherein R² comprises a Fab comprising a heavy chain comprising the amino acid sequence of SEQ ID NO: 19 and a light chain comprising the amino acid sequence of SEQ ID NO: 20;
wherein R¹ is covalently linked at attachment point A to R²; wherein n1 is an integer representing the number of instances of R¹, and wherein each instance of R¹ is covalently linked to a different amino acid residue of the Fab.

46. The complex of claim 45, wherein each of the different amino acid residue is a lysine.

47. The complex of claim 45, wherein the heavy chain of the Fab comprises an N-terminal pyroglutamate.

48. A method of reducing DUX4 expression in a subject, the method comprising administering to the subject a composition comprising the complex of claim 45.

49. The method of claim 48, wherein reducing DUX4 expression comprises reducing DUX4 protein and/or DUX4 mRNA levels.

50. A method of treating facioscapulohumeral muscular dystrophy (FSHD) in a subject, the method comprising administering to the subject a composition comprising the complex of claim 45.

51. The method of claim 50, wherein the subject has aberrant production of DUX4 protein.

52. A complex comprising a structure of the formula (Id): 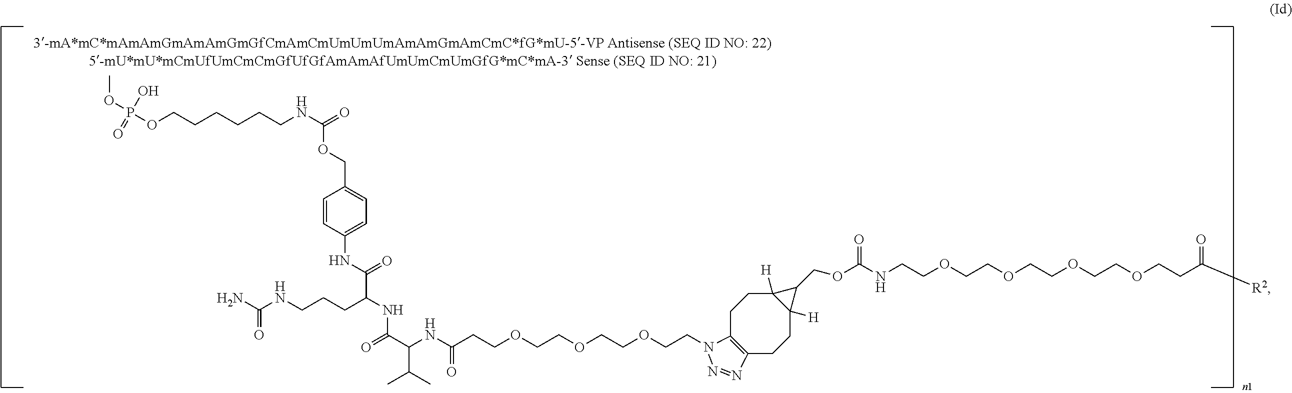
or a pharmaceutically acceptable salt thereof,
wherein: mA, mC, mG, and mU are 2′-O-methyl adenosine, 2′-O-methyl cytidine, 2′-O-methyl guanosine, and 2′-O-methyl uridine, respectively;
fA, fC, fG, and fU are 2′-fluoro adenosine, 2′-fluoro cytidine, 2′-fluoro guanosine, and 2′-fluoro uridine, respectively; “*” between two nucleosides represents a phosphorothioate linkage; the absence of “*” between two nucleosides represents a phosphodiester internucleoside linkage; and “VP” represents 5′-(E)-Vinylphosphonate;
wherein R² comprises a Fab comprising a heavy chain comprising the amino acid sequence of SEQ ID NO: 19 and a light chain comprising the amino acid sequence of SEQ ID NO: 20;
wherein n1 is an integer representing the number of instances of the group enclosed by square brackets, and wherein each instance of the group enclosed by square brackets is covalently linked to a different amino acid residue of the Fab.

53. The complex of claim 52, wherein each of the different amino acid residue is a lysine.

54. The complex of claim 52, wherein the heavy chain of the Fab comprises an N-terminal pyroglutamate.

55. A method of reducing DUX4 expression in a subject, the method comprising administering to the subject a composition comprising the complex of claim 52.

56. The method of claim 55, wherein reducing DUX4 expression comprises reducing DUX4 protein and/or DUX4 mRNA levels.

57. A method of treating facioscapulohumeral muscular dystrophy (FSHD) in a subject, the method comprising administering to the subject a composition comprising the complex of claim 52.

58. The method of claim 57, wherein the subject has aberrant production of DUX4 protein.
